(19) United States
(12) Patent Application Publication
(10) Pub. No.: US 2007/0293486 A1
(43) Pub. Date: Dec. 20, 2007

(54) Alpha-Keto Carbonyl Calpain Inhibitors

(75) Inventors: Philipp Weyermann, Sissach (CH); Andreas Von Sprecher, Oberwil (CH); Marco Hennebohle, Rheinfelden (DE); Holger Herzner, Ruemmingen (DE); Cyrille Lescop, Kembs Loechle (FR); Herve Siendt, Ranspach-le-Haut (FR)

Correspondence Address: KILYK & BOWERSOX, P.L.L.C., 400 HOLIDAY COURT, SUITE 102, WARRENTON, VA, 20186, US

(73) Assignee: SANTHERA PHARMACEUTICALS (SCHWEIZ) AG, Liestal (CH)

(21) Appl. No.: 11/574,095

(22) PCT Filed: Aug. 22, 2005
(86) PCT No.: PCT/EP05/09068

§ 371 Date: Apr. 2, 2007

(30) Foreign Application Priority Data
Aug. 25, 2004 (EP) EP 04020190.7

(51) Int. Cl. A61K 31/535; A61K 31/38; A61K 31/385; C07D 339/02; C07D 413/00; C07D 401/00; C07D 333/10; A61K 31/44

(52) U.S. Cl. 514/231.5; 514/336; 514/440; 514/446; 544/145; 546/268.4; 549/39; 549/78

(57) ABSTRACT

The present invention relates to novel α-keto carbonyl calpain inhibitors for the treatment of neurodegenerative diseases and neuromuscular diseases including Duchenne Muscular Dystrophy, Becker Muscular Dystrophy and other muscular dystrophies. Disuse atrophy and general muscle wasting can also be treated. Diseases of the eye, in particular cataract, can be treated as well. Generally all condition where elevated levels of calpains are involved can be treated. The compounds of the invention may also inhibit other thiol proteases such as cathepsin B, cathepsin H, cathepsin L, papain or the like. Multicatalytic Protease also known as proteasome may also be inhibited and the compounds can therefore be used to treat cell proliferative diseases such as cancer, psoriasis, and restenosis. The compounds of the present invention are also inhibitors of cell damage by oxidative stress through free radicals can be used to treat mitochondrial disorders and neurodegenerative diseases, where elevated levels of oxidative stress are involved. In addition they introduce the expression of utrophin, which is beneficial for the treatment of Duchenne Muscular Dystrophy and Becker Muscular Dystrophy.

### FIELD OF THE INVENTION

The present invention relates to novel α-keto carbonyl calpain inhibitors for the treatment of neurodegenerative diseases and neuromuscular diseases including Duchenne Muscular Dystrophy (DMD), Becker Muscular Dystrophy (BMD) and other muscular dystrophies. Disuse atrophy and general muscle wasting can also be treated. Ischemias of the heart, the kidneys, or of the central nervous system, and cataract and other diseases of the eye can be treated as well. Generally all conditions where elevated levels of calpains are involved can be treated.

The novel calpain inhibitors may also inhibit other thiol proteases, such as cathepsin B, cathepsin H, cathepsin L and papain. Multicatalytic Protease (MCP) also known as proteasome may also be inhibited by the compounds of the invention. The compounds of the present invention can be used to treat diseases related to elevated activity of MCP, such as muscular dystrophy, disuse atrophy, neuromuscular diseases, cardiac cachexia, cancer cachexia, psoriasis, restenosis, and cancer. Generally all conditions where activity of MCP is involved can be treated.

Surprisingly, the compounds of the present invention are also inhibitors of cell damage by oxidative stress through free radicals and can be used to treat mitochondrial disorders and neurodegenerative diseases, where elevated levels of oxidative stress are involved.

Surprisingly, the compounds of the present invention also potently induce the expression of utrophin and can be used to treat disorders and diseases, where elevated levels of utrophin have beneficial therapeutic effects, such as Duchenne Muscular Dystrophy (DMD) and Becker Muscular Dystrophy (BMD).

Also provided are pharmaceutical compositions containing the same.

### BACKGROUND OF THE INVENTION

Neural tissues, including brain, are known to possess a large variety of proteases, including at least two calcium-stimulated proteases, termed calpain I and calpain II.

Calpains are calcium-dependent cysteine proteases present in a variety of tissues and cells and use a cysteine residue in their catalytic mechanism. Calpains are activated by an elevated concentration of calcium, with a distinction being made between calpain I or μ-calpain, which is activated by micromolar concentrations of calcium ions, and calpain II or m-calpain, which is activated by millimolar concentrations of calcium ions (P. Johnson, Int. J. Biochem., 1990, 22(8), 811-22). Excessive activation of calpain provides a molecular link between ischaemia or injury induced by increases in intra-neuronal calcium and pathological neuronal degeneration. If the elevated calcium levels are left uncontrolled, serious structural damage to neurons may result. Recent research has suggested that calpain activation may represent a final common pathway in many types of neurodegenerative diseases. Inhibition of calpain would, therefore, be an attractive therapeutic approach in the treatment of these diseases. Calpains play an important role in various physiological processes including the cleavage of regulatory proteins such as protein kinase C, cytoskeletal proteins such as MAP 2 and spectrin, and muscle proteins, protein degradation in rheumatoid arthritis, proteins associated with the activation of platelets, neuropeptide metabolism, proteins in mitosis and others which are listed in M. J. Barrett et al., Life Sci., 1991, 48, 1659-69 and K. K. Wang et al., Trends in Pharmacol. Sci., 1994, 15, 412-419. Elevated levels of calpain have been measured in various pathophysiological processes, for example: ischemias of the heart (eg. cardiac infarction), of the kidney or of the central nervous system (eg. stroke), inflammations, muscular dystrophies, injuries to the central nervous system (eg. trauma), Alzheimer's disease, etc. (see K. K. Wang, above). These diseases have a presumed association with elevated and persistent intracellular calcium levels, which cause calcium-dependent processes to be overactivated and no longer subject to physiological control. In a corresponding manner, overactivation of calpains can also trigger pathophysiological processes. Exemplary of these diseases would be myocardial ischaemia, cerebral ischaemia, muscular dystrophy, stroke, Alzheimer's disease or traumatic brain injury. Other possible uses of calpain inhibitors are listed in K. K. Wang, Trends in Pharmacol. Sci., 1994, 15, 412-419. It is considered that thiol proteases, such as calpain or cathepsins, take part in the initial process in the collapse of skeletal muscle namely the disappearance of Z line through the decomposition of muscular fiber protein as seen in muscular diseases, such as muscular dystrophy or amyotrophy (Taisha, Metabolism, 1988, 25, 183).

Furthermore, E-64-d, a thiol protease inhibitor, has been reported to have life-prolonging effect in experimental muscular dystrophy in hamster (Journal of Pharmacobiodynamics, 1987, 10, 678). Accordingly, such thiol protease inhibitors are expected to be useful as therapeutic agents, for example, for the treatment of muscular dystrophy or amyotrophy.

An increased level of calcium-mediated proteolysis of essential lens proteins by calpains is also considered to be an important contributor to some forms of cataract of the eyes (S. Biwas et al., Trends in Mol. Med., 2004). Accordingly, calpain inhibitors are expected to be useful as therapeutic agents for the treatment of cataract and are diseases of the eye.

Eukaryotic cells constantly degrade and replace cellular protein. This permits the cell to selectively and rapidly remove proteins and peptides hasting abnormal conformations, to exert control over metabolic pathways by adjusting levels of regulatory peptides, and to provide amino acids for energy when necessary, as in starvation. See Goldberg, A. L. & St. John, A. C. Annu. Rev. Biochem., 1976, 45, 747-803. The cellular mechanisms of mammals allow for multiple pathways for protein breakdown. Some of these pathways appear to require energy input in the form of adenosine triphosphate (“ATP”). See Goldberg & St. John, supra. Multicatalytic protease (MCP, also typically referred to as “multicatalytic proteinase,” “proteasome,” “multicatalytic proteinase complex,” “multicatalytic endopeptidase complex,” “20S proteasome” and “ingensin”) is a large molecular weight (700 kD) eukaryotic non-lysosomal proteinase complex which plays a role in at least two cellular pathways for the breakdown of protein to peptides and amino acids. See Orlowski, M., Biochemistry, 1990, 9(45), 10289-10297. The complex has at least three different types of hydrolytic activities: (1) a trypsin-like activity wherein peptide bonds are cleaved at the carboxyl side of basic amino acids; (2) a chymotrypsin-like activity wherein peptide bonds are cleaved at the carboxyl side of hydrophobic amino acids; and (3) an activity wherein peptide bonds are cleaved at the carboxyl side of glutamic acid. See Rivett, A. J., J. Biol. Chem., 1989, 264(21), 12215-12219 and Orlowski, supra. One route of protein hydrolysis which involves MCP also involves the polypeptide “ubiquitin.” Hershko, A. & Crechanovh, A., Annu. Rev. Biochem., 1982, 51, 335-364. This route, which requires MCP, ATP and ubiquitin, appears responsible for the degradation of highly abnormal proteins, certain short-lived normal proteins and the bulk of proteins in growing fibroblasts and maturing reticuloytes. See Driscoll, J. and Goldberg, A. L., Proc. Nat. Acad. Sci. U.S.A., 1989, 86, 787-791. Proteins to be degraded by this pathway are covalently bound to ubiquitin via their lysine amino groups in an ATP-dependent manner. The ubiquitin-conjugated proteins are then degraded to small peptides by an ATP-dependent protease complex, the 26S proteasome, which contains MCP as its proteolytic core. Goldberg, A. L. & Rock, K. L., Nature, 1992, 357, 375-379. A second route of protein degradation which requires MCP and ATP, but which does not require ubiquitin, has also been described. See Driscoll, J. & Goldberg, A. L., supra. In this process, MCP hydrolyzes proteins in an ATP-dependent manner. See Goldberg, A. L. & Rock, K. L., supra. This process has been observed in skeletal muscle. See Driscoll & Goldberg, supra. However, it has been suggested that in muscle, MCP functions synergistically with another protease, multipain, thus resulting in an accelerated breakdown of muscle protein. See Goldberg & Rock, supra. It has been reported that MCP functions by a proteolytic mechanism wherein the active site nucleophile is the hydroxyl group of the N-terminal threonine residue. Thus, MCP is the first known example of a threonine protease. See Seemuller et al., Science, 1995, 268, 579-582; Goldberg, A. L., Science, 1995, 268, 522-523. The relative activities of cellular protein synthetic and degradative pathways determine whether protein is accumulated or lost. The abnormal loss of protein mass is associated with several disease states such as muscular dystrophy, disuse atrophy, neuromuscular diseases, cardiac cachexia, and cancer cachexia. Accordingly, such MCP inhibitors are expected to be useful as therapeutic agents, for the treatment of these diseases.

Cyclins are proteins that are involved in cell cycle control in eukaryotes. Cyclins presumably act by regulating the activity of protein kinases, and their programmed degradation at specific stages of the cell cycle is required for the transition from one stage to the next. Experiments utilizing modified ubiquitin (Glotzer et al., Nature, 1991, 349, 132; Hershko et al., J. Biol. Chem., 1991, 266, 376) have established that the ubiquitination/proteolysis pathway is involved in cyclin degradation. Accordingly, compounds that inhibit this pathway would cause cell cycle arrest and would be useful in the treatment of cancer, psoriasis, restenosis, and other cell proliferative diseases.

On a cellular level elevated oxidative stress leads to cell damage and mitochondrial disorders such as Kearns-Sayre syndrome, mitochondrial encephalomyopathy-lactic-acidosis-stroke like episodes (MELAS), myoclonic epilepsy and ragged-red-fibers (MERRF), Leber hereditary optic neuropathy (LHON), Leigh's syndrome, neuropathy-ataxia-retinitis pigmentosa (NARP) and progressive external opthalmoplegia (PEO) summarized in Schapira and Griggs (eds) 1999 Muscle Diseases, Butterworth-Heinemann.

Cell damage induced by free radicals is also involved in certain neurodegenerative diseases. Examples for such diseases include degenerative ataxias such as Friedreich' Ataxia, Parkinson's disease, Huntington's disease, amyotrophic lateral sclerosis (ALS), and Alzheimer's disease (Beal M. F., Howell N., Bodis-Wollner I. (eds), 1997, Mitochondria and free radicals in neurodegenerative diseases, Wiley-Liss).

Both Duchenne Muscular Dystrophy (DMD) and Becker Muscular Dystrophy (BMD) are caused by mutations in the dystrophin gene. The dystrophin gene consists of 2700 kbp and is located on the X chromosome (Xp21.2, gene bank accession number: M18533). The 14 kbp long mRNA transcript is expressed predominantly in skeletal, cardiac and smooth muscle and to a limited extent in the brain. The mature dystrophin protein has a molecular weight of ˜427 kDa and belongs to the spectrin superfamily of proteins (Brown S. C., Lucy J. A. (eds), “Dystrophin”, Cambridge University Press, 1997). While the underlying mutation in DMD leads to a lack of dystrophin protein, the milder BMD-phenotype is a consequence of mutations leading to the expression of abnormal, often truncated, forms of the protein with residual functionality. Within the spectrin superfamily of proteins, dystrophin is closest related to utrophin (gene bank accession number: X69086), to dystrophin related protein-2 (gene bank accession number: NM001939) and to dystrobrevin (gene bank accession number: dystrobrevin alpha: BC005300, dystrobrevin beta: BT009805). Utrophin is encoded on chromosome 6 and the ˜395 kDa utrophin protein is ubiquitously expressed in a variety of tissues including muscle cells. The N-terminal part of utrophin protein is 80% identical to that of dystrophin protein and binds to actin with similar affinity. Moreover, the C-terminal region of utrophin also binds to β-dystroglycan, α-dystrobrevin and syntrophins.

Utrophin is expressed throughout the muscle cell surface during embryonic development and is replaced by dystrophin during postembryonic development. In adult muscle utrophin protein is confined to the neuromuscular junction. Thus, in addition to sequence and structural similarities between dystrophin and utrophin, both proteins share certain cellular functions. Consequently, it has been proposed that upregulation of utrophin could ameliorate the progressive muscle loss in DMD and BMD patients and offers a treatment option for this devastating disease (WO96/34101). Accordingly, compounds that induce the expression of utrophin could be useful in the treatment of DMD and BMD (Tinsley, J. M., Potter, A. C., et al., Nature, 1996, 384, 349; Yang, L., Lochmuller, H., et al., Gene Ther.; 1998, 5, 369; Gilbert, R., Nalbantoglu, J., et al., Hum. Gene Ther. 1999, 10, 1299).

Calpain inhibitors have been described in the literature. However, these are predominantly either irreversible inhibitors or peptide inhibitors. As a rule, irreversible inhibitors are alkylating substances and suffer from the disadvantage that they react nonselectively in the organism or are unstable. Thus, these inhibitors often have undesirable side effects, such as toxicity, and are therefore of limited use or are unusable. Examples of the irreversible inhibitors are E-64 epoxides (E. B. McGowan et al., Biochem. Biophys. Res. Commun., 1989, 158, 432-435), alpha-haloketones (H. Angliker et al., J. Med. Chem., 1992, 35, 216-220) and disulfides (R. Matsueda et al., Chem. Lett., 1990, 191-194).

Many known reversible inhibitors of cysteine proteases, such as calpain, are peptide aldehydes, in particular dipeptide or tripeptide aldehydes, such as Z-Val-Phe-H (MDL 28170) (S. Mehdi, Trends in Biol. Sci., 1991, 16, 150-153), which are highly susceptible to metabolic inactivation.

It is the object of the present invention to provide novel α-keto carbonyl calpain inhibitors preferentially acting in muscle cells in comparison with known calpain inhibitors.

In addition, the calpain inhibitors of the present invention may have a unique combination of other beneficial properties such as proteasome (MCP) inhibitory activity and/or protection of muscle cells from damage due to oxidative stress and/or induction of utrophin expression. Such properties could be advantageous for treating muscular dystrophy and amyotrophy.

### SUMMARY OF THE INVENTION

The present invention relates to novel α-keto carbonyl calpain inhibitors of the formula (I) and their tautomeric and isomeric forms, and also, where appropriate, physiologically tolerated salts.

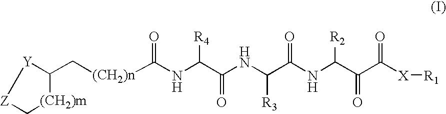

These α-keto carbonyl compounds are effective in the treatment of neurodegenerative diseases and neuromuscular diseases including Duchenne Muscular Dystrophy (DMD), Becker Muscular Dystrophy (BMD) and other muscular dystrophies. Disuse atrophy and general muscle wasting can also be treated. Ischemias of the heart, the kidneys, or of the central nervous system, and cataract and other diseases of the eye can be treated as well. Generally, all conditions where elevated levels of calpains are involved can be treated.

The compounds of the invention may also inhibit other thiol proteases, such as cathepsin B, cathepsin H, cathepsin L and papain. Multicatalytic Protease (MCP) also known as proteasome may also be inhibited, which is beneficial for the treatment of muscular dystrophy. Proteasome inhibitors can also be used to treat cancer, psoriasis, restenosis, and other cell proliferative diseases.

Surprisingly, the compounds of the present invention are also inhibitors of cell damage by oxidative stress through free radicals and can be used to treat mitochondrial disorders and neurodegenerative diseases, where elevated levels of oxidative stress are involved.

Surprisingly, the compounds of the present invention also potently induce the expression of utrophin and can be used to treat disorders and diseases, where elevated levels of utrophin have beneficial therapeutic effects, such as Duchenne Muscular Dystrophy (DMD) and Becker Muscular Dystrophy (BMD).

The present invention also relates to pharmaceutical compositions comprising the compounds of the present invention and a pharmaceutically acceptable carrier.

### DETAILED DESCRIPTION OF THE INVENTION

The present invention relates to novel α-keto carbonyl calpain inhibitors of the formula (I) and their tautomeric and isomeric forms, and also, where appropriate, physiologically tolerated salts, where the variables have the following meanings: 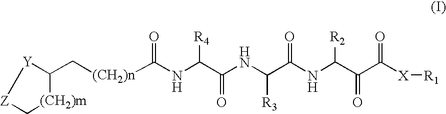
R¹ represents

hydrogen,

straight chain alkyl,

branched chain alkyl

cycloalkyl,

-alkylene-cycloalkyl,

aryl,

-alkylene-aryl,

—SO₂-alkyl,

—SO₂-aryl,

-alkylene-SO₂-aryl,

-alkylene-SO₂-alkyl,

heterocyclyl or

-alkylene-heterocyclyl;

—CH₂CO—X—H

—CH₂CO—X-straight chain alkyl,

—CH₂CO—X-branched chain alkyl,

—CH₂CO—X-cycloalkyl,

—CH₂CO—X-alkylene-cycloalkyl,

—CH₂CO—X-aryl,

—CH₂CO—X-alkylene-aryl,

—CH₂CO—X-heterocyclyl,

—CH₂CO—X-alkylene-heterocyclyl or

—CH₂CO-aryl;

X represents O or NH;

R² represents

hydrogen,

straight chain alkyl,

branched chain alkyl,

cycloalkyl,

-alkylene-cycloalkyl,

aryl or

-alkylene-aryl;

R³ represents

hydrogen,

straight chain alkyl,

branched chain alkyl,

cycloalkyl or

-alkylene-cycloalkyl;

R⁴ represents

straight chain alkyl,

branched chain alkyl,

cycloalkyl,

-alkylene-cycloalkyl,

aryl,

-alkylene-aryl or

-alkenylene-aryl;

wherein each of m and n represents an integer of 0 to 6, i.e. 1, 2, 3, 4, 5 or 6;

Y and Z independently represents

S,

SO or

CH₂.

In the present invention, the substituents attached to formula (I) are defined as follows:

An alkyl group is a straight chain alkyl group, a branched chain alkyl group or a cycloalkyl group as defined below.

A straight chain alkyl group means a group —(CH₂)ₓCH₃, wherein x is 0 or an integer of 1 or more. Preferably, x is 0 or an integer of 1 to 9, i.e. 1, 2, 3, 4, 5, 6, 7, 8 or 9, i.e the straight chain alkyl group has 1 to 10 carbon atoms. More preferred, x is 0 or an integer of 1 to 6, i.e. 1, 2, 3, 4, 5 or 6. Examples of the straight chain alkyl group are methyl, ethyl, n-propyl, n-butyl, n-pentyl, n-hexyl, n-heptyl, n-octyl, n-nonyl and n-decyl.

A branched chain alkyl group contains at least one secondary or tertiary carbon atom. For example, the branched chain alkyl group contains one, two or three secondary or tertiary carbon atoms. In the present invention, the branched chain alkyl group preferably has at least 3 carbon atoms, more preferably 3 to 10, i.e. 3, 4, 5, 6, 7, 8, 9 or 10, carbon atoms, further preferred 3 to 6 carbon atoms, i.e. 3, 4, 5 or 6 carbon atoms. Examples thereof are iso-propyl, sec.-butyl, tert.-butyl, 1,1-dimethyl propyl, 1,2-dimethyl propyl, 2,2-dimethyl propyl(neopentyl), 1,1-dimethyl butyl, 1,2-dimethyl butyl, 1,3-dimethyl butyl, 2,2-dimethyl butyl, 2,3-dimethyl butyl, 3,3-dimethyl butyl, 1-ethyl butyl, 2-ethyl butyl, 3-ethyl butyl, 1-n-propyl propyl, 2-n-propyl propyl, 1-iso-propyl propyl, 2-iso-propyl propyl, 1-methyl pentyl, 2-methyl pentyl, 3-methyl pentyl and 4-methyl pentyl.

In the present invention, a cycloalkyl group preferably has 3 to 8 carbon atoms, i.e. 3, 4, 5, 6, 7 or 8 carbon atoms. Examples thereof are cyclopropyl, cyclobutyl, cyclopentyl, cyclohexyl, cycloheptyl and cyclooctyl. More preferably, the cycloalkyl group has 3 to 6 carbon atoms, such as cyclopentyl, cyclohexyl and cycloheptyl.

In the present invention, the straight chain or branched chain alkyl group or cycloalkyl group may be substituted with at least one halogen atom selected from the group consisting of F, Cl, Br and I, among which F is preferred. Preferably, 1 to 5 hydrogen atoms of said straight chain or branched chain alkyl group or cycloalkyl group have been replaced by halogen atoms. Preferred haloalkyl groups include —CF₃, —CH₂CF₃ and —CF₂CF₃.

In the present invention, an alkoxy group is an —O-alkyl group, wherein alkyl is as defined above.

In the present invention, an alkylamino group is an —NH-alkyl group, wherein alkyl is as defined above.

In the present invention, a dialkylamino group is an —N(alkyl)₂ group, wherein alkyl is as defined above and the two alkyl groups may be the same or different.

In the present invention, an acyl group is a —CO-alkyl group, wherein alkyl is as defined above.

In an alkyl-O—CO— group, alkyl-O—CO—NH— group and alkyl-S— group, alkyl is as defined above.

An alkylene moiety may be a straight chain or branched chain group. Said alkylene moiety preferably has 1 to 6, i.e. 1, 2, 3, 4, 5 or 6, carbon atoms. Examples thereof include methylene, ethylene, n-propylene, n-butylene, n-pentylene, n-hexylene, methyl methylene, ethyl methylene, 1-methyl ethylene, 2-methyl ethylene, 1-ethyl ethylene, propyl methylene, 2-ethyl ethylene, 1-methyl propylene, 2-methyl propylene, 3-methyl propylene, 1-ethyl propylene, 2-ethyl propylene, 3-ethyl propylene, 1,1-dimethyl propylene, 1,2-dimethyl propylene, 2,2-dimethyl propylene, 1,1-dimethyl butylene, 1,2-dimethyl butylene, 1,3-dimethyl butylene, 2,2-dimethyl butylene, 2,3-dimethyl butylene, 3,3-dimethyl butylene, 1-ethyl butylene, 2-ethyl butylene, 3-ethyl butylene, 4-ethyl butylene, 1-n-propyl propylene, 2-n-propyl propylene, 1-iso-propyl propylene, 2-iso-propyl propylene, 1-methyl pentylene, 2-methyl pentylene, 3-methyl pentylene, 4-methyl pentylene and 5-methyl pentylene. More preferably, said alkylene moiety has 1 to 4 carbon atoms, such as methylene, ethylene, n-propylene, 1-methyl ethylene and 2-methyl ethylene.

In the present invention, a cycloalkylene group preferably has 3 to 8 carbon atoms, i.e. 3, 4, 5, 6, 7 or 8 carbon atoms. Examples thereof are cyclopropylene, cyclobutylene, cyclopentylene, cyclohexylene, cycloheptylene and cyclooctylene. More preferably, the cycloalkylene group has 3 to 6 carbon atoms, such as cyclopropylene, cyclobutylene, cyclopentylene, and cyclohexylene. In the cycloalkylene group, the two bonding positions may be at the same or at adjacent carbon atoms or 1, 2 or 3 carbon atoms are between the two bonding positions. In preferred cycloalkylene groups the two bonding positions are at the same carbon atom or 1 or 2 carbon atoms are between the two bonding positions.

An alkenylene group is a straight chain or branched alkenylene moiety having preferably 2 to 8 carbon atoms, more preferably 2 to 4 atoms, and at least one double bond, preferably one or two double bonds, more preferably one double bond. Examples thereof are vinylene, allylene, methallylene, buten-2-ylene, buten-3-ylene, penten-2-ylene, penten-3-ylene, penten-4-ylene, 3-methyl-but-3-enylene, 2-methyl-but-3-enylene, 1-methyl-but-3-enylene, hexenylene or heptenylene.

An aryl group is a carbocyclic or heterocyclic aromatic mono- or polycyclic moiety. The carbocyclic aromatic mono- or polycyclic moiety preferably has at least 6 carbon atoms, more preferably 6 to 20 carbon atoms. Examples thereof are phenyl, biphenyl, naphthyl, tetrahydronaphthyl, fluorenyl, indenyl and phenanthryl among which phenyl and naphthyl are preferred. Phenyl is especially preferred. The heterocyclic aromatic monocyclic moiety is preferably a 5- or 6-membered ring containing carbon atoms and at least one heteroatom, for example 1, 2 or 3 heteroatoms, such as N, O and/or S. Examples thereof are thienyl, pyridyl, furanyl, pyrrolyl, thiophenyl, thiazolyl and oxazolyl, among which thienyl and pyridyl are preferred. The heterocyclic aromatic polycyclic moiety is preferably an aromatic moiety having 6 to 20 carbon atoms with at least one heterocycle attached thereto. Examples thereof are benzothienyl, naphthothienyl, benzofuranyl, chromenyl, indolyl, isoindolyl, indazolyl, quinolyl, isoquinolyl, phthalazinyl, quinaxalinyl, cinnolinyl and quinazolinyl.

The aryl group may have 1, 2, 3, 4 or 5 substituents, which may be the same or different. Examples of said substituents are straight chain or branched chain alkyl groups as defined above, halogen atoms, such as F, Cl, Br or I, hydroxy groups, alkyloxy groups, wherein the alkyl moiety is as defined above, fluoroalkyl groups, i.e. alkyl groups as defined above, wherein I to (2x+3) hydrogen atoms are substituted by fluoro atoms, especially trifluoro methyl, —COOH groups, —COO-alkyl groups and —CONH-alkyl groups, wherein the alkyl moiety is as defined above, nitro groups, and cyano groups.

An arylene group is a carbocyclic or heterocyclic aromatic mono- or polycyclic moiety attached to two groups of a molecule. In the monocyclic arylene group, the two bonding positions may be at adjacent carbon atoms or 1 or 2 carbon atoms are between the two bonding positions. In the preferred monocyclic arylene groups 1 or 2 carbon atoms are between the two bonding positions. In the polycyclic arylene group, the two bonding positions may be at the same ring or at different rings. Further, they may be at adjacent carbon atoms or 1 or more carbon atoms are between the two bonding positions. In the preferred polycyclic arylene groups I or more carbon atoms are between the two bonding positions. The carbocyclic aromatic mono- or polycyclic moiety preferably has at least 6 carbon atoms, more preferably 6 to 20 carbon atoms. Examples thereof are phenylene, biphenylene, naphthylene, tetrahydronaphthalene, fluorenylene, indenylene and phenanthrylene among which phenylene and naphthylene are preferred. Phenylene is especially preferred. The heterocyclic aromatic monocyclic moiety is preferably a 5- or 6-membered ring containing carbon atoms and at least one heteroatom, for example 1, 2 or 3 heteroatoms, such as N, O and/or S. Examples thereof are thienylene, pyridylene, furanylene, pyrrolylene, thiophenylene, thiazolylene and oxazolyiene, among which thienylene and pyridylene are preferred. The heterocyclic aromatic polycyclic moiety is preferably an aromatic moiety having 6 to 20 carbon atoms with at least one heterocycle attached thereto. Examples thereof are benzothienylene, naphthothienylene, benzofuranylene, chromenylene, indolylene, isoindolylene, indazolylene, quinolylene, isoquinolylene, phthalazinylene, quinaxalinylene, cinnolinylene and quinazolinylene.

The arylene group may have 1, 2, 3, 4 or 5 substituents, which may be the same or different. Examples of said substituents are straight chain or branched chain alkyl groups as defined above, halogen atoms, such as F, Cl, Br or I, alkyloxy groups, wherein the alkyl moiety is as defined above, fluoroalkyl groups, i.e. alkyl groups a defined above, wherein 1 to (2x+3) hydrogen atoms are substituted by fluoro atoms, especially trifluoro methyl.

The heterocyclyl group is a saturated or unsaturated non-aromatic ring containing carbon atoms and at least one hetero atom, for example 1, 2 or 3 heteroatoms, such as N, O and/or S. Examples thereof are morpholinyl, piperidinyl, piperazinyl and imidazolinyl.

In formula (I), R¹ may be hydrogen.

In formula (I), R¹ may be a straight chain alkyl group as defined above. In the more preferred straight chain alkyl group x is 0 or an integer of 1 to 3, i.e. the straight chain alkyl group of R¹ is preferably selected from methyl, ethyl, n-propyl and n-butyl. Especially preferred, the straight chain alkyl group is ethyl.

In formula (I), R¹ may be a branched chain alkyl group as defined above. The more preferred branched chain alkyl group has 3 or 4 carbon atoms, examples thereof being iso-propyl, sec.-butyl, and tert.-butyl. Especially preferred, the branched chain chain alkyl group is iso-propyl.

In formula (I), R¹ may be a cycloalkyl group as defined above. The more preferred cycloalkyl group is cyclopropyl.

In formula (I), R¹ may be an -alkylene-cycloalkyl group. Therein, the alkylene moiety and the cycloalkyl group are as defined above.

In formula (I), R¹ may be an aryl group as defined above. The more preferred aryl group is mono- or bicyclic aryl. Especially preferred, the aryl group is phenyl or pyridyl.

In formula (I), R¹ may be an -alkylene-aryl group. Therein, the alkylene moiety and the aryl group are as defined above. More preferred, the alkylene moiety contains 1 to 4 carbon atoms. The more preferred aryl group attached to an alkylene moiety is mono- or bicyclic aryl. Especially preferred, the aryl group is phenyl or pyridyl.

In formula (I), R¹ may be an SO₂-alkyl group, wherein alkyl is as defined above.

In formula (I), R¹ may be an SO₂-aryl group, wherein aryl is as defined above.

In formula (I), R¹ may be an -alkylene-SO₂-aryl group, wherein alkylene and aryl are as defined above. More preferred, the alkylene moiety contains 1 to 4 carbon atoms. The more preferred aryl group attached to the SO₂-moiety is mono- or bicyclic aryl. Especially preferred, the aryl group is phenyl or pyridyl.

In formula (I), R¹ may be an -alkylene-SO₂-alkyl group, wherein alkylene and alkyl are as defined above. More preferred, the alkylene moiety contains 1 to 4 carbon atoms.

In formula (I), R¹ may be a heterocyclyl group as defined above.

In formula (I), R¹ may be an -alkylene-heterocyclyl group, wherein the alkylene moiety and the heterocyclyl group are as defined above. More preferred, the alkylene moiety contains 1 to 4 carbon atoms. The more preferred heterocyclyl group attached to an alkylene moiety is monocyclic heterocyclyl. Especially preferred, the heterocyclyl group is morpholinyl.

In formula (I), R¹ may be —CH₂COOH or —CH₂CONH₂.

In formula (I), R¹ may be a —CH₂CO—X-straight chain alkyl group. Therein, the straight chain alkyl group is as defined above. In the more preferred straight chain alkyl group x is 0 or an integer of 1 to 3, i.e. the straight chain alkyl group of R¹ is preferably selected from methyl, ethyl, n-propyl and n-butyl.

In formula (I), R¹ may be a —CH₂CO—X-branched chain alkyl group. Therein, the branched chain alkyl group is as defined above. The more preferred branched chain alkyl group has 3 or 4 carbon atoms, examples thereof being iso-propyl, sec.-butyl, and tert.-butyl. Especially preferred, the branched chain chain alkyl group is iso-propyl.

In formula (I), R¹ may be a —CH₂CO—X-cycloalkyl group. Therein, the cycloalkyl group is as defined above.

In formula (I), R¹ may be an —CH₂CO—X-alkylene-cycloalkyl group. Therein, the alkylene moiety and the cycloalkyl group are as defined above.

In formula (I), R¹ may be a —CH₂CO—X-aryl group. Therein, the aryl group is as defined above. The more preferred aryl group is mono- or bicyclic aryl. Especially preferred, the aryl group is phenyl or pyridyl.

In formula (I), R¹ may be an —CH₂CO—X-alkylene-aryl group. Therein, the alkylene moiety and the aryl group are as defined above. More preferred, the alkylene moiety contains 1 to 4 carbon atoms. The more preferred aryl group attached to an alkylene moiety is mono- or bicyclic aryl. Especially preferred, the aryl group is phenyl or pyridyl.

In formula (I), R¹ may be a —CH₂CO—X-heterocyclyl group. Therein, the heterocyclyl group is as defined above.

In formula (I), R¹ may be an —CH₂CO—X-alkylene-heterocyclyl group, wherein the alkylene moiety and the heterocyclyl group are as defined above. More preferred, the alkylene moiety contains 1 to 4 carbon atoms. The more preferred heterocyclyl group attached to an alkylene moiety is monocyclic heterocyclyl. Especially preferred, the heterocyclyl group is morpholinyl.

In formula (I), R¹ may be a —CH₂CO-aryl group. Therein, the aryl group is as defined above. The more preferred aryl group is mono- or bicyclic aryl. Especially preferred, the aryl group is phenyl or pyridyl.

Preferably, R¹ is selected from the group consisting of hydrogen, straight chain alkyl, branched chain alkyl, cycloalkyl, -alkylene-aryl, and -alkylene-heterocyclyl, —CH₂CO—X-straight chain alkyl, —CH₂COOH and —CH₂CONH₂. More preferably, R¹ is hydrogen, straight chain alkyl or cycloalkyl. Most preferably, R¹ is ethyl.

In formula (I), R² may be a straight chain alkyl group as defined above.

In formula (I), R² may be a branched chain alkyl group as defined above. More preferred, the branched chain alkyl group has 3 or 4 carbon atoms, examples thereof being iso-propyl, sec.-butyl and 1-methyl-propyl. Especially preferred is sec.-butyl.

In formula (I), R² may be an aryl group as defined above. The more preferred aryl group is an optionally substituted phenyl group having one or two substituents. Preferred substituents are selected from the group consisting of halogen atoms, especially F and/or Cl and/or Br, alkyl groups, especially methyl, alkyloxy groups, especially methoxy or ethoxy, fluoroalkyl groups, such as trifluoromethyl, and nitro and cyano groups.

In formula (I), R² may be an -alkylene-aryl group. Therein, the alkylene moiety and the aryl group are as defined above. More preferred, the alkylene moiety is a methylene group. The more preferred aryl group attached to the alkylene moiety is an optionally substituted phenyl group having one or two substituents. Preferred substituents are selected from the group consisting of halogen atoms, especially F and/or Cl and/or Br, alkyl groups, especially methyl, alkyloxy groups, especially methoxy or ethoxy, fluoroalkyl groups, such as trifluoromethyl, and nitro and cyano groups. Especially preferred substituents are F, Cl, Br, methyl, and methoxy.

Preferably, R² is a substituted or unsubstituted benzyl group. More preferably, R² is a substituted benzyl group, having one or two substituents selected from the group consisting of halogen atoms, alkyl groups, fluoroalkyl groups and alkyloxy groups. Most preferably, R² is a substituted benzyl group, having one or two substituents selected from the group consisting of F, Cl, Br, methyl, and methoxy.

In formula (I), R³ may be a straight chain alkyl group as defined above.

In formula (I), R³ may be a branched chain alkyl group as defined above. More preferred, the branched chain alkyl group has 3 or 4 carbon atoms, examples thereof being iso-propyl, sec.-butyl and 1-methyl-propyl. Especially preferred is iso-propyl and sec.-butyl.

In formula (I), R³ may be a cycloalkyl group as defined above. The preferred cycloalkyl group is cyclopropyl.

In formula (I), R³ may be an -alkylene-cycloalkyl group. Therein, the alkylene moiety and the cycloalkyl group are as defined above. The preferred alkylene moiety is a methylene group. The preferred cycloalkyl group is cyclopropyl.

Preferably, R³ is a branched chain alkyl group, a cycloalkyl group, or an -alkylene-cycloalkyl group as defined above. More preferably, R³ is a branched chain alkyl group as defined above. Most preferably, R³ is iso-propyl or sec.-butyl.

In formula (I), R⁴ may be a straight chain alkyl group as defined above.

In formula (I), R⁴ may be a branched chain alkyl group as defined above. More preferred, the branched chain alkyl group has 3 or 4 carbon atoms, examples thereof being iso-propyl, sec.-butyl and 1-methyl-propyl. Especially preferred is sec.-butyl.

In formula (I), R⁴ may be a cycloalkyl group as defined above. The preferred cycloalkyl group is cyclopropyl.

In formula (I), R⁴ may be an aryl group as defined above. The more preferred aryl group is an optionally substituted phenyl group having one or two substituents. Preferred substituents are selected from the group consisting of halogen atoms, especially F and/or Cl and/or Br, alkyl groups, especially methyl, alkyloxy groups, especially methoxy or ethoxy, fluoroalkyl groups, such as trifluoromethyl, and nitro and cyano groups.

In formula (I), R⁴ may be an -alkylene-cycloalkyl group. Therein, the alkylene moiety and the aryl group are as defined above. More preferred, the alkylene moiety is a methylene group. The more preferred cycloalkyl group is a 5-7 membered ring. Especially preferred is cyclohexyl.

In formula (I), R⁴ may be an -alkylene-aryl group. Therein, the alkylene moiety and the aryl group are as defined above. More preferred, the alkylene moiety is a methylene or ethylene group. The more preferred aryl group attached to the alkylene moiety is an optionally substituted phenyl group having one or two substituents or a naphthyl or pyridyl group. Preferred substituents are selected from the group consisting of halogen atoms, especially F and/or Cl and/or Br, alkyl groups, especially methyl, alkyloxy groups, especially methoxy or ethoxy, fluoroalkyl groups, such as trifluoromethyl, and nitro and cyano groups. Especially preferred substituents are F, Cl, Br, methyl, and methoxy.

In formula (I), R⁴ may be an -alkenylene-aryl group. Therein, the alkenylene moiety and the aryl group are as defined above. More preferred, the alkenylene moiety is a vinylene or allylene group. The more preferred aryl group attached to the alkenylene moiety is an optionally substituted phenyl group having one or two substituents or a naphthyl or pyridyl group. Preferred substituents are selected from the group consisting of halogen atoms, especially F and/or Cl and/or Br, alkyl groups, especially methyl, alkyloxy groups, especially methoxy or ethoxy, fluoroalkyl groups, such as trifluoromethyl, and nitro and cyano groups. Especially preferred substituents are F, Cl, Br, methyl, and methoxy.

Preferably, R⁴ is a substituted or unsubstituted benzyl or ethylphenyl group, or a methylnaphthyl group.

In formula (I), m and n are as defined above. More preferred, m is an integer of 1-2. More preferred, n is an integer of 1-4. Especially preferred, m is 1 and/or n is 3.

In formula (I), Y and Z are as defined above. More preferred, and Y and Z independently represent S or SO. Especially preferred, Y and Z are both S or Y is S and Z is SO or Y is SO and Z is S.

Preferably, m is an integer of 1-2, n is an integer of 1-4, and Y and Z independently represent S or SO. In this case it is more preferred that Y and Z are both S or Y is S and Z is SO or Y is SO and Z is S. Even more preferably, m is 1, n is 3, and Y and Z are both S or Y is S and Z is SO or Y is SO and Z is S. Most preferably, m is 1, n is 3, and Y and Z are both S.

The compounds of structural formula (I) are effective calpain inhibitors and may also inhibit other thiol proteases, such as cathepsin B, cathepsin H, cathepsin L or papain. Multicatalytic Protease (MCP) also known as proteasome may also be inhibited. The compounds of formula (I) are particularly effective as calpain inhibitors and are therefore useful for the treatment and/or prevention of disorders responsive to the inhibition of calpain, such as neurodegenerative diseases and neuromuscular diseases including Duchenne Muscular Dystrophy (DMD), Becker Muscular Dystrophy (BMD) and other muscular dystrophies, like disuse atrophy and general muscle wasting and other diseases with the involvement of calpain, such as ischemias of the heart, the kidneys or of the central nervous system, cataract, and other diseases of the eyes.

Optical Isomers—Diastereomers—Geometric Isomers—Tautomers

The compounds of structural formula (I) contain one or more asymmetric centers and can occur as racemates and racemic mixtures, single enantiomers, diastereomeric mixtures and individual diastereomers. The present invention is meant to comprehend all such isomeric forms of the compounds of structural formula (I).

Some of the compounds described herein may exist as tautomers such as keto-enol tautomers. The individual tautomers as well as mixtures thereof are encompassed within the compounds of structural formula (I).

The compounds of structural formula (I) may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general formula (I) may be obtained by stereospecific synthesis using optically pure starting materials or reagents of known absolute configuration.

Salts

The term “pharmaceutically acceptable salts” refers to salts prepared from pharmaceutically acceptable non-toxic bases or acids including inorganic or organic bases and inorganic or organic acids. Salts derived from inorganic bases include, for example, aluminum, ammonium, calcium, copper, ferric, ferrous, lithium, magnesium, manganic, manganous, potassium, sodium and zinc salts. Particularly preferred are the ammonium, calcium, lithium, magnesium, potassium and sodium salts. Salts derived from pharmaceutically acceptable organic non-toxic bases include salts of primary, secondary and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine and tromethamine.

When the compound of the present invention is basic, salts may be prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, parnoic, pantothenic, phosphoric, propionic, succinic, sulfuric, tartaric, p-toluenesulfonic and trifluoroacetic acid. Particularly preferred are citric, fumaric, hydrobromic, hydrochloric, maleic, phosphoric, sulfuric and tartaric acid.

It will be understood that, as used herein, references to the compounds of formula (I) are meant to also include the pharmaceutically acceptable salts.

Utility

The compounds of formula (I) are calpain inhibitors and as such are useful for the preparation of a medicament for the treatment, control or prevention of diseases, disorders or conditions responsive to the inhibition of calpain such as neurodegenerative diseases and neuromuscular diseases including Duchenne Muscular Dystrophy (DMD), Becker Muscular Dystrophy (BMD) and other muscular dystrophies. Neuromuscular diseases such as muscular dystrophies, include dystrophinopathies and sarcoglycanopathies, limb girdle muscular dystrophies, congenital muscular dystrophies, congenital myopathies, distal and other myopathies, myotonic syndromes, ion channel diseases, malignant hyperthermia, metabolic myopathies, hereditary cardiomyopathies, congenital myasthenic syndromes, spinal muscular atrophies, hereditary ataxias, hereditary motor and sensory neuropathies, hereditary paraplegias, and other neuromuscular disorders, as defined in Neuromuscular Disorders, 2003, 13, 97-108. Disuse atrophy and general muscle wasting can also be treated. Generally all conditions where elevated levels of calpains are involved can be treated, including, for example, ischemias of the heart (eg. cardiac infarction), of the kidney or of the central nervous system (eg. stroke), inflammations, muscular dystrophies, cataracts of the eye and other diseases of the eyes, injuries to the central nervous system (eg. trauma) and Alzheimer's disease.

The compounds of formula (I) may also inhibit other thiol proteases such as, cathepsin B, cathepsin H, cathepsin L and papain. Multicatalytic Protease (MCP) also known as proteasome may also be inhibited by the compounds of the invention and as such they are useful for the preparation of a medicament for the treatment, control or prevention of diseases, disorders or conditions responsive to the inhibition of MCP such as muscular dystrophy, disuse atrophy, neuromuscular diseases, cardiac cachexia, and cancer cachexia. Cancer, psoriasis, restenosis, and other cell proliferative diseases can also be treated.

Surprisingly, the compounds of formula (I) are also inhibitors of cell damage by oxidative stress through free radicals and as such they are useful for the preparation of a medicament for the treatment of mitochondrial disorders and neurodegenerative diseases, where elevated levels of oxidative stress are involved.

Mitochondrial disorders include Keams-Sayre syndrome, mitochondrial encephalomyopathy-lactic-acidosis-stroke like episodes (MELAS), myoclonic epilepsy and ragged-red-fibers (MERRF), Leber hereditary optic neuropathy (LHON), Leigh's syndrome, neuropathy-ataxia-retinitis pigmentosa (NARP) and progressive external opthalmoplegia (PEO) summarized in Schapira and Griggs (eds) 1999 Muscle Diseases, Butterworth-Heinemann.

Neurodegenerative diseases with free radical involvement include degenerative ataxias, such as Friedreich' Ataxia, Parkinson's disease, Huntington's disease, amyotrophic lateral sclerosis (ALS) and Alzheimer's disease (Beal M. F., Howell N., Bodis-Wollner I. (eds), 1997, Mitochondria and free radicals in neurodegenerative diseases, Wiley-Liss).

Surprisingly, the compounds of formula (I) also potently induce the expression of utrophin and as such they are useful for the preparation of a medicament for the treatment of diseases, disorders or conditions, where elevated levels of utrophin have beneficial therapeutic effects, such as Duchenne Muscular Dystrophy (DMD) and Becker Muscular Dystrophy (BMD).

Administration and Dose Ranges

Any suitable route of administration may be employed for providing a mammal, especially a human, with an effective dosage of a compound of the present invention. For example, oral, rectal, topical, parenteral, ocular, pulmonary or nasal administration may be employed. Dosage forms include, for example, tablets, troches, dispersions, suspensions, solutions, capsules, creams, ointments and aerosols. Preferably the compounds of formula (I) are administered orally, parenterally or topically.

The effective dosage of the active ingredient employed may vary depending on the particular compound employed, the mode of administration, the condition being treated and the severity of the condition being treated. Such dosage may be ascertained readily by a person skilled in the art.

When treating Duchenne Muscular Dystrophy (DMD), Becker Muscular Dystrophy (BMD) and other muscular dystrophies, generally, satisfactory results are obtained when the compounds of the present invention are administered at a daily dosage of about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating ischemias of the heart (eg. cardiac infarction), of the kidney or of the central nervous system (eg. stroke), generally, satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating cancer, psoriasis, restenosis, and other cell proliferative diseases, generally, satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating mitochondrial disorders or neurodegenerative diseases where oxidative stress is a factor, generally, satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

Formulation

The compound of formula (I) is preferably formulated into a dosage form prior to administration. Accordingly the present invention also includes a pharmaceutical composition comprising a compound of formula (I) and a suitable pharmaceutical carrier.

The present pharmaceutical compositions are prepared by known procedures using well-known and readily available ingredients. In making the formulations of the present invention, the active ingredient (a compound of formula (I)) is usually mixed with a carrier, or diluted by a carrier, or enclosed within a carrier, which may be in the form of a capsule, sachet, paper or other container. When the carrier serves as a diluent, it may be a solid, semisolid or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders.

Some examples of suitable carriers, excipients and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents and/or flavoring agents. The compositions of the invention may be formulated so as to provide quick, sustained or delayed release of the active ingredient after administration to the patient

Preparation of Compounds of the Invention

The compounds of formula (I) of the present invention can be prepared according to the procedures of the following Schemes and Examples, using appropriate materials and are further exemplified by the following specific examples. Moreover, by utilizing the procedures described herein in conjunction with ordinary skills in the art additional compounds of the present invention can be readily prepared. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius.

When describing the preparation of the present compounds of formula (I), the terms “T moiety”, “Amino acid (AA) moiety” and “Dipeptide moiety” are used below. This moiety concept is illustrated below:

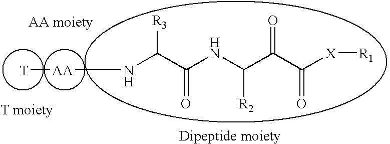

The preparation of the compounds of the present invention may be advantageously carried out via sequential synthetic routes. The skilled artisan will recognize that in general, the three moieties of a compound of formula (I) are connected via amide bonds. The skilled artisan can, therefore, readily envision numerous routes and methods of connecting the three moieties via standard peptide coupling reaction conditions.

The phrase “standard peptide coupling reaction conditions” means coupling a carboxylic acid with an amine using an acid activating agent such as EDC, dicyclohexylcarbodiimide, and benzotriazol-1-yl-N,N,N′,N′-tetramethyluronium hexafluorophosphate in a inert solvent such as DMF in the presence of a catalyst such as HOBt. The uses of protective groups for amine and carboxylic acids to facilitate the desired reaction and minimize undesired reactions are well documented. Conditions required to remove protecting groups which may be present can be found in Greene, et al., Protective Groups in Organic Synthesis, John Wiley & Sons, Inc., New York, N.Y. 1991.

Protecting groups like Z, Boc and Fmoc are used extensively in the synthesis, and their removal conditions are well known to those skilled in the art. For example, removal of Z groups can he achieved by catalytic hydrogenation with hydrogen in the presence of a noble metal or its oxide such as palladium on activated carbon in a protic solvent such as ethanol. In cases where catalytic hydrogenation is contraindicated by the presence of other potentially reactive functionality, removal of Z can also be achieved by treatment with a solution of hydrogen bromide in acetic acid, or by treatment with a mixture of TFA and dimethylsulfide. Removal of Boc protecting groups is carried out in a solvent such as methylene chloride, methanol or ethyl acetate with a strong acid, such as TFA or HCl or hydrogen chloride gas. Fmoc protecting groups can be removed with piperidine in a suitable solvent such as DMF.

The required dipeptide moieties can advantageously be prepared via a Passerini reaction (T. D. Owens et al., Tet. Lett., 2001, 42, 6271; L. Banfi et al., Tet. Lett., 2002, 43, 4067) from an R¹-isonitrile, a suitably protected R²-aminoaldehyde, and a suitably protected R³-amino acid followed by N-deprotection and acyl-migration, which leads to the corresponding dipeptidyl α-hydroxy-amide. The groups R¹, R² and R³ are as defined above with respect to formula (I). The reactions are carried out in an inert solvent such as CH₂Cl₂ at room temperature. The α-keto amide functionality on the dipeptide moiety is typically installed using a Dess-Martin oxidation (S. Chatterjee et al., J. Med. Chem., 1997, 40, 3820) in an inert solvent such as CH₂Cl₂ at 0° C. or room temperature. This oxidation can be carried out either following the complete assembly of the compounds of Formula (I) using peptide coupling reactions or at any convenient intermediate stage in the sequence of connecting the three moieties T, M, and dipeptide, as it will be readily recognized by those skilled in the art.

The compounds of formula (I), when existing as a diastereomeric mixture, may be separated into diastereomeric pairs of enantiomers by fractional crystallization from a suitable solvent such as methanol, ethyl acetate or a mixture thereof. The pair of enantiomers thus obtained may be separated into individual stereoisomers by conventional means by using an optically active acid as a resolving agent. Alternatively, any enantiomer of a compound of the formula (I) may be obtained by stereospecific synthesis using optically pure starting materials or reagents of known configuration.

In the above description and in the schemes, preparations and examples below, the various reagent symbols and abbreviations have the following meanings:

1-Nal 1-naphthylalanine

2-Nal 2-naphthylalanine

Boc t-butoxycarbonyl

DIEA diisopropylethylamine

DMAP 4-dimethylaminopyridine

DMF N,N-dimethylformamide

EDC 1-(3-dimethylaminopropyl)-3-ethylcarbodiimide hydrochloride

Et ethyl

EtOAc ethyl acetate

Fmoc 9-fluorenylmethyl-carbamate

HBTU benzotriazol-1-yl-N,N,N′,N′-tetramethyluronium hexafluorophosphate

HOAc acetic acid

HOAt 1-hydroxy-7-azabenzotriazole

HOBt 1-hydroxybenzotriazole

h hour(s)

Homophe homophenylalanine

Leu leucine

Me methyl

NMM N-methylmorpholine

Phe phenylalanine

Py pyridyl

PyBOP benzotriazol-1-yloxytris(pyrrolidino)-phosphonium hexafluorophosphate

TFA trifluoroacetic acid

TEA triethylamine

Val valine

z benzyloxycarbonyl

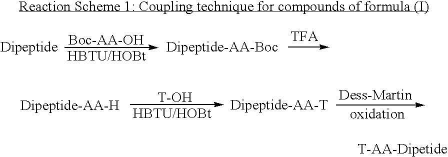

An appropriate dipeptide moiety (e.g. H₂N-Val-Phe(4-Cl)-hydroxy-ethylamide) is coupled to an M moiety (e.g. Boc-Phe-OH) in the presence of HBTU/HOBt followed by Boc deprotection. The coupled M-dipeptide hydroxy-ethylamide compound is then coupled to an appropriate T moiety (e.g. Lipoic acid) followed by Dess-Martin oxidation to the corresponding α-keto amide compound.

Generally, after a peptide coupling reaction is completed, the reaction mixture can be diluted with an appropriate organic solvent, such as EtOAc, CH₂Cl₂ or Et₂O, which is then washed with aqueous solutions, such as water, HCl, NaHSO₄, bicarbonate, NaH₂PO₄, phosphate buffer (pH 7), brine or any combination thereof. The reaction mixture can be concentrated and then be partitioned between an appropriate organic solvent and an aqueous solution. The reaction mixture can be concentrated and subjected to chromatography without aqueous workup.

Protecting groups such as Boc, Z, Fmoc and CF₃CO can be deprotected in the presence of H₂/Pd—C, TFA/DCM, HCl/EtOAc, HCl/doxane, HCl in MeOH/Et₂O, NH₃/MeOH or TBAF with or without a cation scavenger, such as thioanisole, ethane thiol and dimethyl sulfide (DMS). The deprotected amines can be used as the resulting salt or are freebased by dissolving in DCM and washing with aqueous bicarbonate or aqueous NaOH. The deprotected amines can also be freebased by ion exchange chromatography.

More detailed procedures for the assembly of compounds of formula (I) are described in the section with the examples of the present invention.

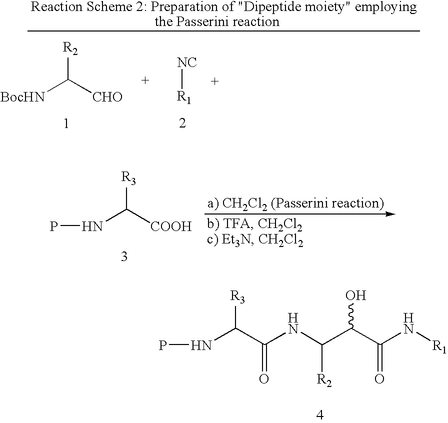

P is an amino protecting group as described before; and R¹ to R³ are as defined above with respect to formula (I).

The dipeptide moieties of the present invention, in general, may be prepared from commercially available starting materials via known chemical transformations. The preparation of a dipeptide moiety of the compound of the present invention is illustrated in the reaction scheme above.

As shown in Reaction Scheme 2, the “dipeptide moiety” of the compounds of the present invention can be prepared by a three-component reaction between a Boc-protected amino aldehyde 1, an isonitrile 2 and a suitably protected amino acid 3 (Passerini reaction) in an organic solvent, such as CH₂Cl₂, at a suitable temperature. Following deprotection of the Boc group using TFA in a suitable solvent, such as CH₂Cl₂, the dipeptide moieties 4 are obtained after base-induced acyl-migration using a suitable base, such as Et₃N or DIEA, in a suitable solvent, such as CH₂Cl₂. More detailed examples of dipeptide moiety preparation are described below.

Suitably functionalized AA moieties are commercially available.

Suitably functionalized T moieties are commercially available or can readily be prepared by the skilled artisan from commercial precursors by published procedures (G. Claeson et al., Arkiv foer Kemi, 1969, 31, 83).

The following describes the detailed examples of the invention.

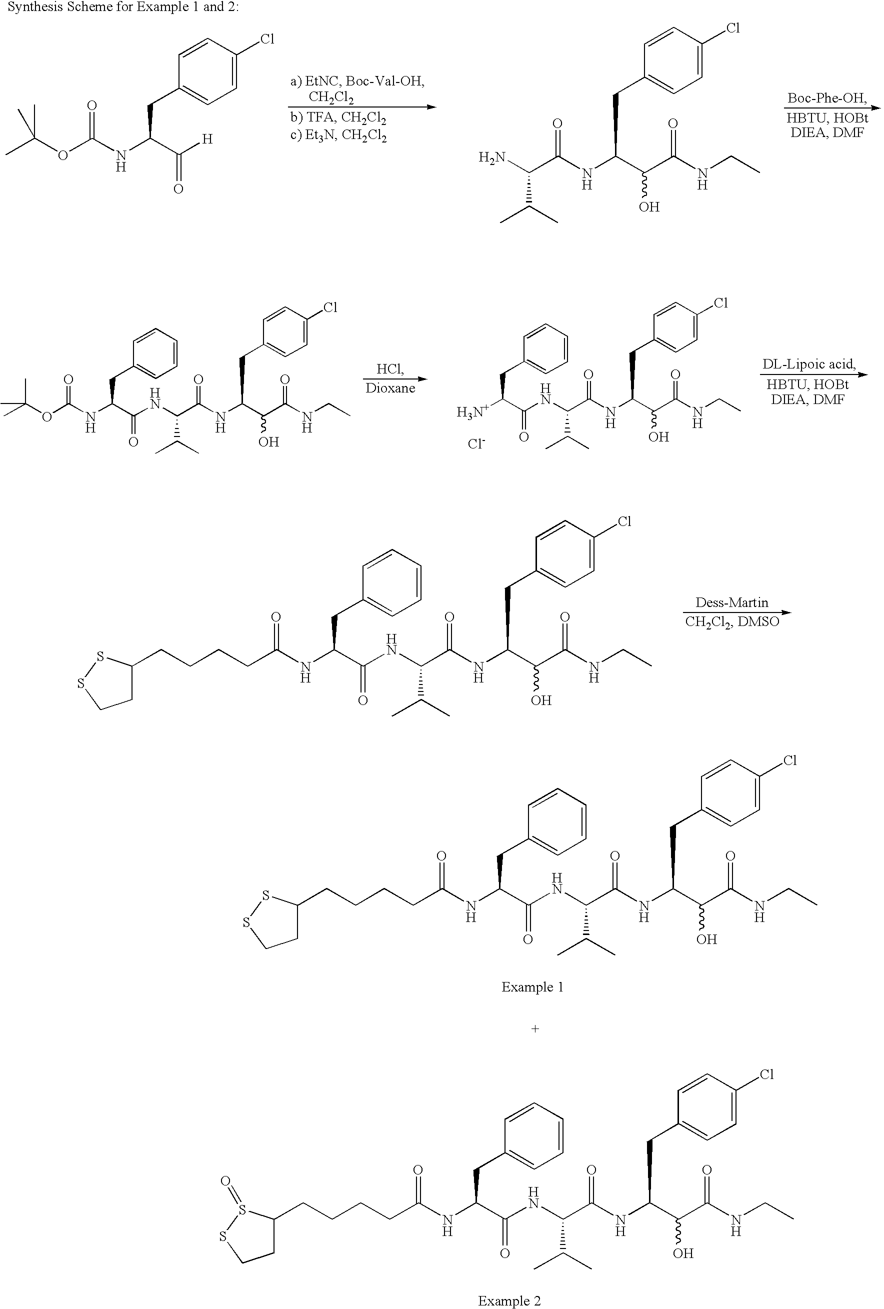

### EXAMPLE 1

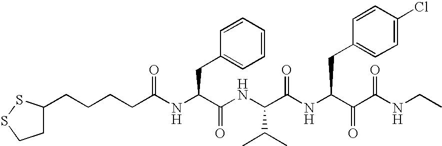

A solution of 555 mg of intermediate 1d) in 3 ml of DMSO and 20 ml of CH₂Cl₂ was cooled in ice. 430 mg of Dess-Martin reagent were added and the mixture was stirred at r.t. for 120 min. CH₂Cl₂ was added and the mixture was washed with 1 M Na₂S₂O₃, sat. NaHCO₃, and H₂O, dried with anh. Na₂SO₄ and evaporated in vacuo. The crude product was purified by column chromatography (CH₂Cl₂/MeOH 98:2→CH₂Cl₂/MeOH 95:5) which yielded Example 1 in form of a slightly yellowish solid. In addition, a smaller amount of Example 2 was obtained as a colorless solid.

R_{f}=0.73 (CH₂Cl₂/MeOH 9:1); Mp. 239-240° C.

The required intermediates can be synthesized in the following way:

Intermediate 1a):

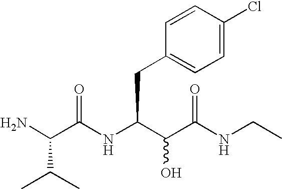

To a solution of 1.00 g of Boc-p-chloro-phenylalaninal in 14 ml of anh. CH₂Cl₂ were added 0.39 ml of Ethyl isocyanide, followed by 0.76 g of Boc-valine, and the mixture was stirred at r.t. for 18 h. The resulting solution was evaporated to dryness and the residue redissolved in 14 ml of CH₂Cl₂. 5 ml of TFA were added and the reaction was stirred at r.t. for 2 h. The volatiles were evaporated in vacuo and the residue dried in vacuo. The resulting yellow oil was dissolved in 14 ml of CH₂Cl₂, 10 ml of Et₃N were added and the reaction was stirred at r.t. overnight. Then the reaction mixture was evaporated to dryness in vacuo and the residue was partitioned between 1 N NaOH and EtOAc. The organic layer was washed with 1 N NaOH, H₂O, and brine. The aqueous layers were back extracted with EtOAc and the combined organic layer dried over Na₂SO₄ and evaporated in vacuo. The crude product was suspended in Et₂O, filtered off, washed with cold Et₂O, and dried in vacuo to yield intermediate 1a) as a white solid.

R_{f}=0.27 (CH₂Cl₂/MeOH 9:1); Mp. 187-190° C.

Intermediate 1b):

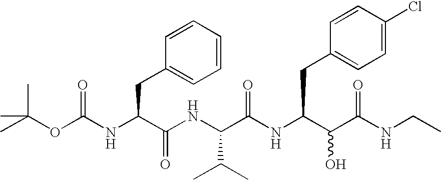

To a solution of 540 mg of Boc-Phe-OH and 363 mg of HOBt in 12 ml of DMF were added 768 mg of HBTU, followed by 0.705 ml of DIEA, and the mixture was stirred at r.t for 10 min. Then, 600 mg of intermediate 1a) were added and the reaction was stirred at r.t. overnight. The resulting solution was diluted with EtOAc, washed with 1 N HCl (3×), 2 N K₂CO₃ (3×), H₂O, and brine. The organic layer was dried with anh. MgSO₄ and evaporated in vacuo. The crude product was triturated with hot Et₂O, filtered off, washed with cold Et₂O, and dried in vacuo to yield intermediate 1b) as a white solid.

R_{f}=0.53 (CH₂Cl₂/MeOH 9:1); Mp. 245-246° C.

Intermediate 1c):

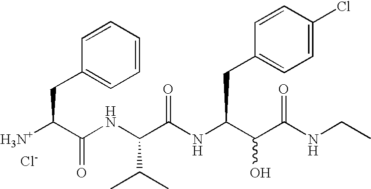

To a solution of 1000 mg of intermediate 1b) in 3 ml of MeOH were added 18 ml of 4 M HCl in dioxane and the clear solution was stirred at r.t. for 120 min. Then, the reaction mixture was diluted with 54 ml of Et₂O and cooled in the fridge for 60 min.

The precipitated product was filtered off, washed with Et₂O, and dried in vacuo at 40° C. overnight to yield intermediate 1c) as a white solid.

R_{f}=0.43 (CH₂Cl₂/MeOH 9:1).

Intermediate 1d):

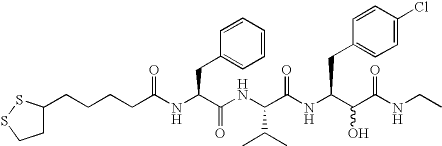

To a ice-cooled solution of 206 mg of DL-Lipoic acid and 204 mg of HOBt in 12 ml of DMF were added 379 mg of HBTU, followed by 0.350 ml of DIEA, and the mixture was stirred in an ice bath for 10 min. Then, 450 mg of intermediate 1c) were added and the reaction was stirred at r.t. overnight. The resulting solution was diluted with EtOAc, washed with 1 N HCl (3×), 2 N K₂CO₃ (3×), H₂O, and brine. The organic layer was dried with anh. MgSO₄ and evaporated in vacuo. The crude product was triturated with hot Et₂O, filtered off, washed with cold Et₂O, and dried in vacuo to yield intermediate 1d) as a yellowish solid.

R_{f}=0.49 (CH₂Cl₂/MeOH 9:1); Mp. 259-261° C.

### EXAMPLE 2

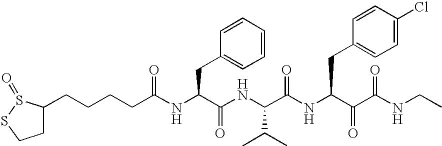 R_{f}=0.47 (CH₂Cl₂₁MeOH 9:1); Mp. 221-231° C.

The compounds of the following examples can be prepared in a similar way:

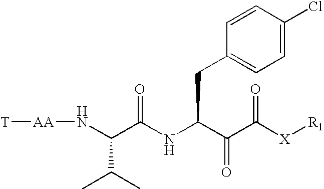

| | | | | | TLC | Mp. |
|---|---|---|---|---|---|---|
| Ex | T | AA | X | R₁ | [R_{f}(Solv.)] | [° C.] |
| 3 | 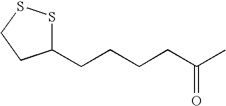 | Phe | O | H | | |
| 4 | 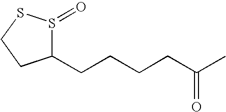 | Phe | O | H | | |
| 5 | 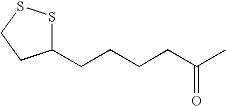 | Phe | O | Me | | |
| 6 | 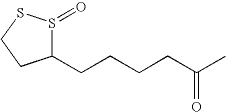 | Phe | O | Me | | |
| 7 | 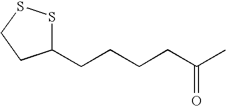 | Phe | NH | 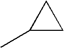 | | |
| 8 | 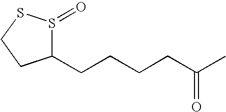 | Phe | NH | 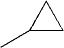 | | |
| 9 | 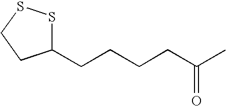 | Phe | NH | CH₂COPh | | |
| 10 | 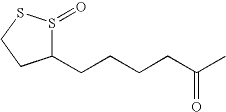 | Phe | NH | CH₂COPh | | |
| 11 | 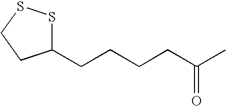 | Phe | NH | 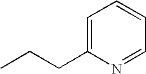 | | |
| 12 | 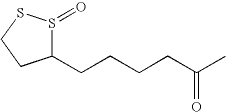 | Phe | NH | 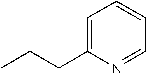 | | |
| 13 | 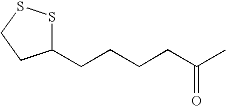 | Phe | NH | 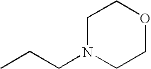 | | |
| 14 | 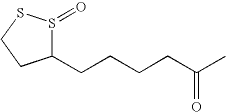 | Phe | NH | 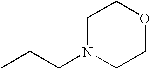 | | |
| 15 | 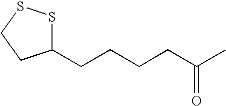 | Phe | NH | CH₂CONH₂ | | |
| 16 | 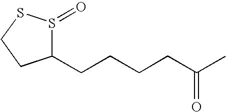 | Phe | NH | CH₂CONH₂ | | |
| 17 | 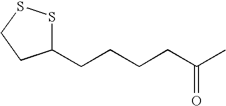 | Phe | NH | CH₂COOEt | | |
| 18 | 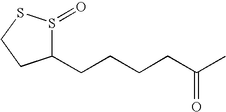 | Phe | NH | CH₂COOEt | | |
| 19 | 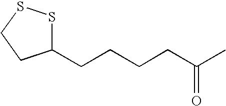 | Phe | NH | CH₂COOH | | |
| 20 | 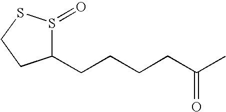 | Phe | NH | CH₂COOH | | |
| 21 | 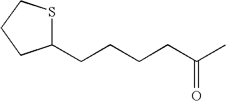 | Phe | NH | Et | | |
| 22 | 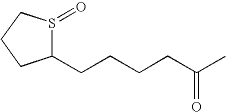 | Phe | NH | Et | | |
| 23 | 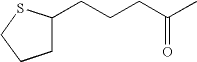 | Phe | NH | Et | | |
| 24 | 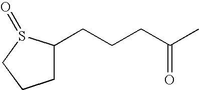 | Phe | NH | Et | | |
| 25 | 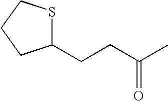 | Phe | NH | Et | | |
| 26 | 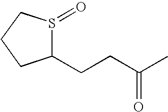 | Phe | NH | Et | | |
| 27 | 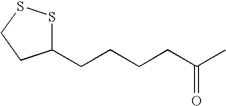 | 1-Nal | NH | Et | | |
| 28 | 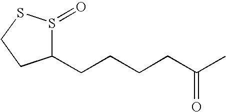 | 1-Nal | NH | Et | 0.48 (CH₂Cl₂/MeOH 9:1) | 226-231 |
| 29 | 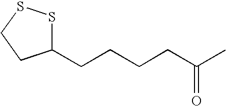 | 1-Nal | O | H | | |
| 30 | 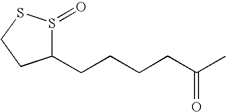 | 1-Nal | O | H | | |
| 31 | 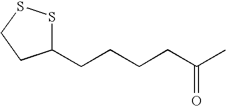 | 1-Nal | O | Me | | |
| 32 | 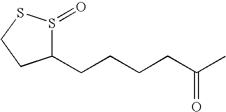 | 1-Nal | O | Me | | |
| 33 | 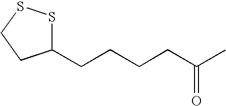 | 1-Nal | NH | 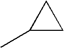 | | |
| 34 | 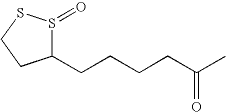 | 1-Nal | NH | 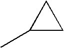 | | |
| 35 | 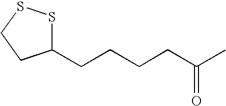 | 1-Nal | NH | CH₂COPh | | |
| 36 | 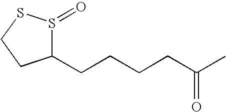 | 1-Nal | NH | CH₂COPh | | |
| 37 | 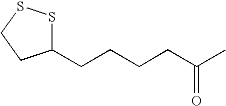 | 1-Nal | NH | 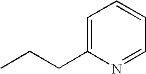 | | |
| 38 | 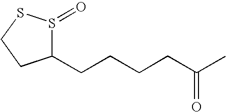 | 1-Nal | NH | 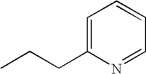 | | |
| 39 | 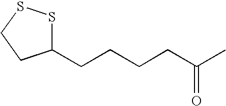 | 1-Nal | NH | 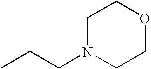 | | |
| 40 | 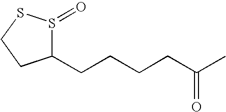 | 1-Nal | NH | 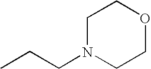 | | |
| 41 | 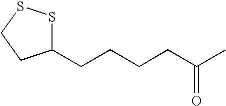 | 1-Nal | NH | CH₂CONH₂ | | |
| 42 | 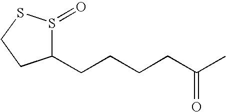 | 1-Nal | NH | CH₂CONH₂ | | |
| 43 | 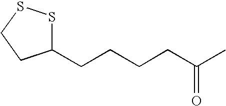 | 1-Nal | NH | CH₂COOEt | | |
| 44 | 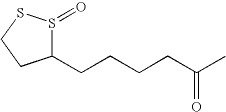 | 1-Nal | NH | CH₂COOEt | | |
| 45 | 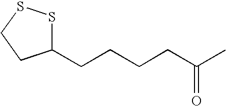 | 1-Nal | NH | CH₂COOH | | |
| 46 | 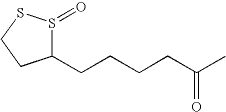 | 1-Nal | NH | CH₂COOH | | |
| 47 | 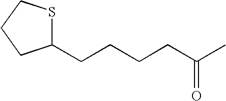 | 1-Nal | NH | Et | | |
| 48 | 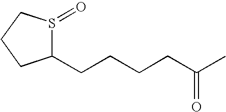 | 1-Nal | NH | Et | | |
| 49 | 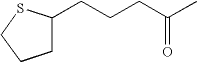 | 1-Nal | NH | Et | | |
| 50 | 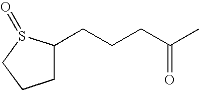 | 1-Nal | NH | Et | | |
| 51 | 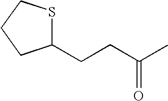 | 1-Nal | NH | Et | | |
| 52 | 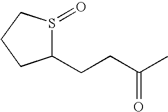 | 1-Nal | NH | Et | | |
| 53 | 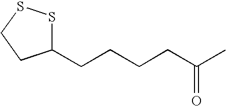 | 2-Nal | NH | Et | | |
| 54 | 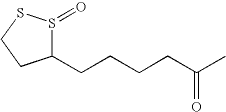 | 2-Nal | NH | Et | 0.48 (CH₂Cl₂/MeOH 9:1) | 228-232 |
| 55 | 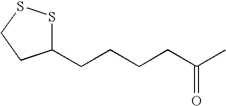 | 2-Nal | O | H | | |
| 56 | 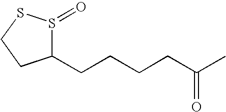 | 2-Nal | O | H | | |
| 57 | 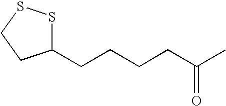 | 2-Nal | O | Me | | |
| 58 | 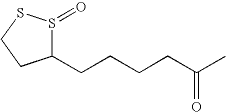 | 2-Nal | O | Me | | |
| 59 | 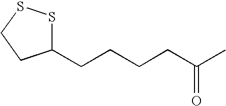 | 2-Nal | NH | 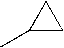 | | |
| 60 | 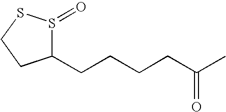 | 2-Nal | NH | 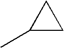 | | |
| 61 | 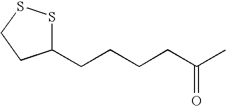 | 2-Nal | NH | CH₂COPh | | |
| 62 | 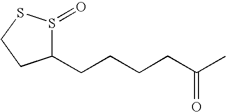 | 2-Nal | NH | CH₂COPh | | |
| 63 | 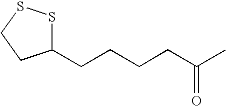 | 2-Nal | NH | 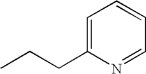 | | |
| 64 | 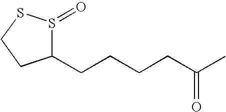 | 2-Nal | NH | 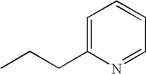 | | |
| 65 | 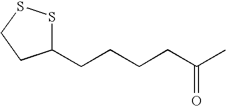 | 2-Nal | NH | 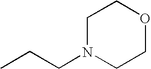 | | |
| 66 | 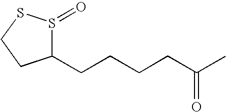 | 2-Nal | NH | 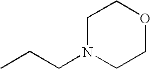 | | |
| 67 | 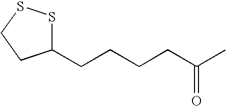 | 2-Nal | NH | CH₂CONH₂ | | |
| 68 | 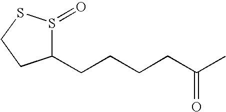 | 2-Nal | NH | CH₂CONH₂ | | |
| 69 | 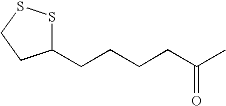 | 2-Nal | NH | CH₂COOEt | | |
| 70 | 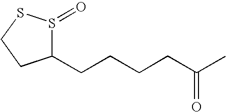 | 2-Nal | NH | CH₂COOEt | | |
| 71 | 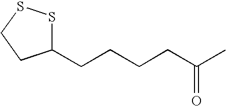 | 2-Nal | NH | CH₂COOH | | |
| 72 | 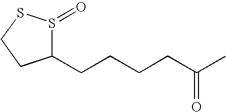 | 2-Nal | NH | CH₂COOH | | |
| 73 | 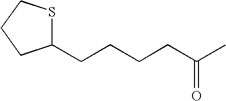 | 2-Nal | NH | Et | | |
| 74 | 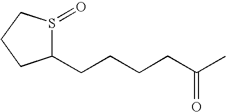 | 2-Nal | NH | Et | | |
| 75 | 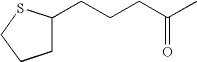 | 2-Nal | NH | Et | | |
| 76 | 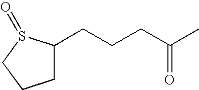 | 2-Nal | NH | Et | | |
| 77 | 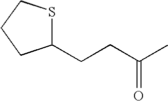 | 2-Nal | NH | Et | | |
| 78 | 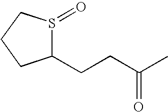 | 2-Nal | NH | Et | | |
| 79 | 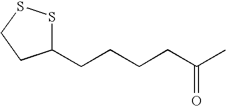 | Homophe | NH | Et | 0.60 (CH₂Cl₂/MeOH 9:1) | 231-232 |
| 80 | 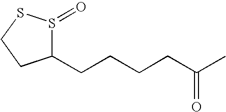 | Homophe | NH | Et | | |
| 81 | 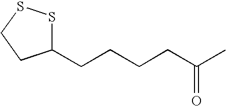 | Homophe | O | H | | |
| 82 | 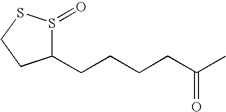 | Homophe | O | H | | |
| 83 | 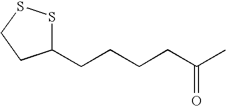 | Homophe | O | Me | | |
| 84 | 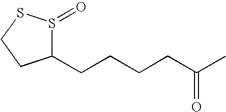 | Homophe | O | Me | | |
| 85 | 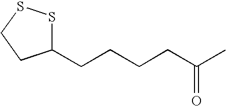 | Homophe | NH | 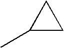 | | |
| 86 | 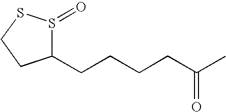 | Homophe | NH | 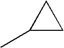 | | |
| 87 | 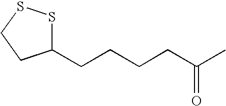 | Homophe | NH | CH₂COPh | | |
| 88 | 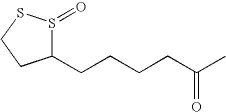 | Homophe | NH | CH₂COPh | | |
| 89 | 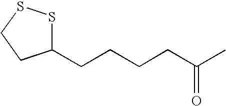 | Homophe | NH | 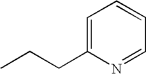 | | |
| 90 | 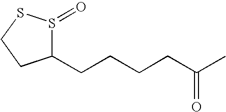 | Homophe | NH | 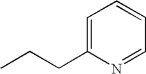 | | |
| 91 | 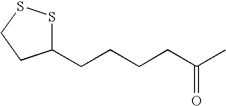 | Homophe | NH | 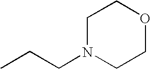 | | |
| 92 | 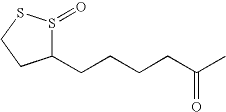 | Homophe | NH | 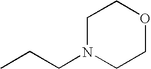 | | |
| 93 | 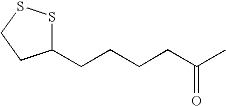 | Homophe | NH | CH₂CONH₂ | | |
| 94 | 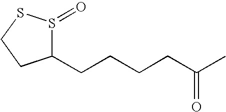 | Homophe | NH | CH₂CONH₂ | | |
| 95 | 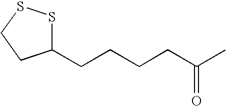 | Homophe | NH | CH₂COOEt | | |
| 96 | 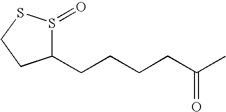 | Homophe | NH | CH₂COOEt | | |
| 97 | 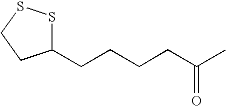 | Homophe | NH | CH₂COOH | | |
| 98 | 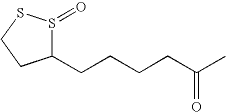 | Homophe | NH | CH₂COOH | | |
| 99 | 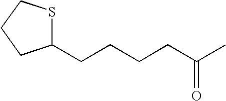 | Homophe | NH | Et | | |
| 100 | 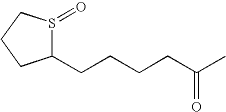 | Homophe | NH | Et | | |
| 101 | 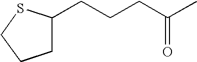 | Homophe | NH | Et | | |
| 102 | 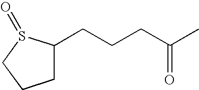 | Homophe | NH | Et | | |
| 103 | 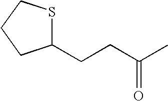 | Homophe | NH | Et | | |
| 104 | 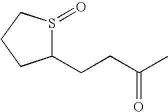 | Homophe | NH | Et | | |
| 105 | 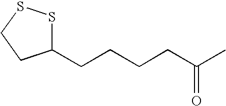 | Phe(4-F) | NH | Et | | |
| 106 | 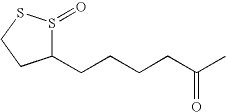 | Phe(4-F) | NH | Et | | |
| 107 | 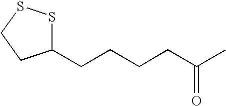 | Phe(4-Cl) | NH | Et | | |
| 108 | 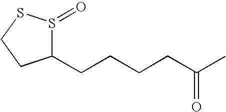 | Phe(4-Cl) | NH | Et | | |
| 109 | 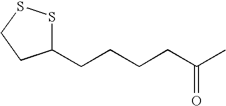 | Phe(3,4-Cl₂) | NH | Et | | |
| 110 | 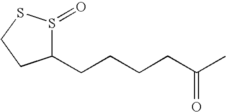 | Phe(3,4-Cl₂) | NH | Et | | |
| 111 | 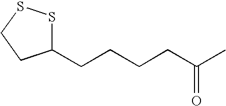 | Phe(4-OMe) | NH | Et | | |
| 112 | 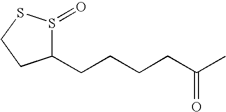 | Phe(4-OMe) | NH | Et | | |
| 113 | 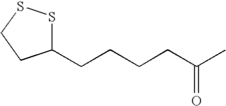 | 3-PyAla | NH | Et | | |
| 114 | 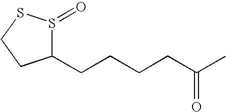 | 3-PyAla | NH | Et | | |
| 115 | 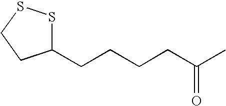 | 3-Benzo- thienylAla | NH | Et | | |
| 116 | 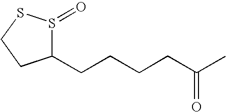 | 3-Benzo- thienylAla | NH | Et | | |
| 117 | 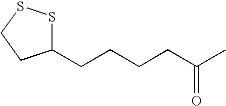 | CyclohexylAla | NH | Et | | |
| 118 | 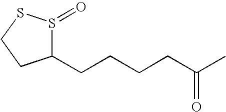 | CyclohexylAla | NH | Et | | |
| 119 | 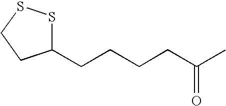 | Leu | NH | Et | | |
| 120 | 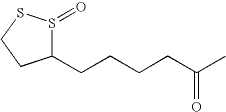 | Leu | NH | Et | | |

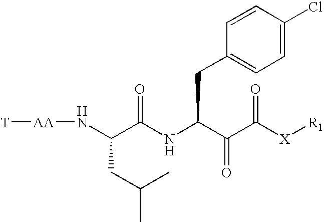

| | | | | | TLC | Mp. |
|---|---|---|---|---|---|---|
| Ex | T | AA | X | R₁ | [R_{f}(Solv.)] | [° C.] |
| 121 | 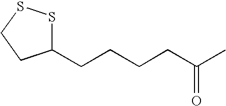 | Phe | NH | Et | 0.55 (CH₂Cl₂/MeOH 9:1) | 205-206 |
| 122 | 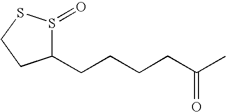 | Phe | NH | Et | | |
| 123 | 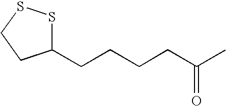 | Phe | O | H | | |
| 124 | 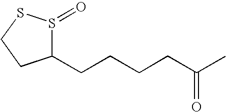 | Phe | O | H | | |
| 125 | 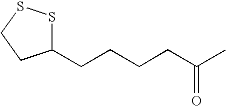 | Phe | O | Me | | |
| 126 | 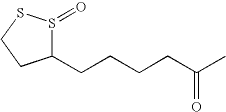 | Phe | O | Me | | |
| 127 | 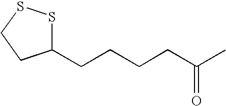 | Phe | NH | 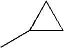 | | |
| 128 | 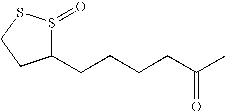 | Phe | NH | 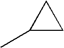 | | |
| 129 | 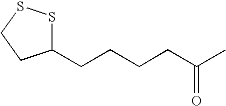 | Phe | NH | CH₂COPh | | |
| 130 | 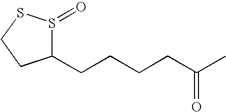 | Phe | NH | CH₂COPh | | |
| 131 | 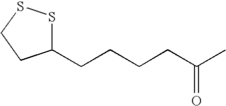 | Phe | NH | 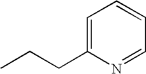 | | |
| 132 | 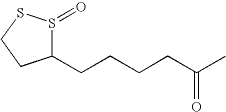 | Phe | NH | 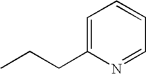 | | |
| 133 | 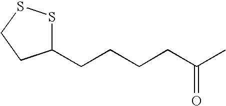 | Phe | NH | 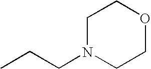 | | |
| 134 | 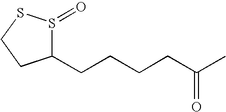 | Phe | NH | 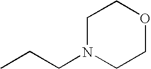 | | |
| 135 | 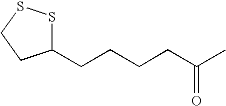 | Phe | NH | CH₂CONH₂ | | |
| 136 | 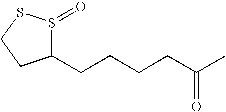 | Phe | NH | CH₂CONH₂ | | |
| 137 | 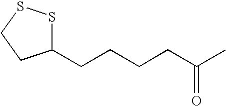 | Phe | NH | CH₂COOEt | | |
| 138 | 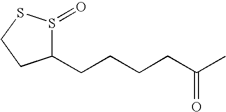 | Phe | NH | CH₂COOEt | | |
| 139 | 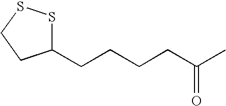 | Phe | NH | CH₂COOH | | |
| 140 | 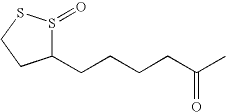 | Phe | NH | CH₂COOH | | |
| 141 | 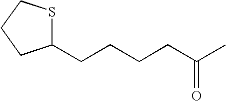 | Phe | NH | Et | | |
| 142 | 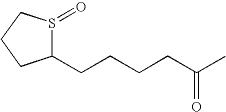 | Phe | NH | Et | | |
| 143 | 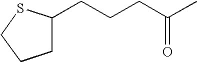 | Phe | NH | Et | | |
| 144 | 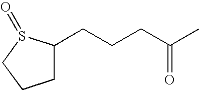 | Phe | NH | Et | | |
| 145 | 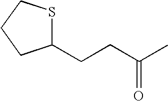 | Phe | NH | Et | | |
| 146 | 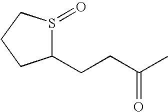 | Phe | NH | Et | | |
| 147 | 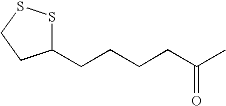 | 1-Nal | NH | Et | | |
| 148 | 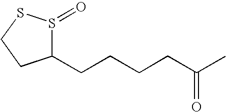 | 1-Nal | NH | Et | | |
| 149 | 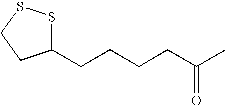 | 1-Nal | O | H | | |
| 150 | 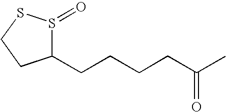 | 1-Nal | O | H | | |
| 151 | 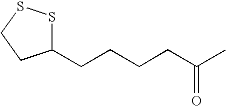 | 1-Nal | O | Me | | |
| 152 | 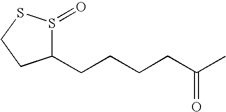 | 1-Nal | O | Me | | |
| 153 | 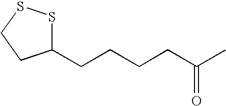 | 1-Nal | NH | 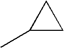 | | |
| 154 | 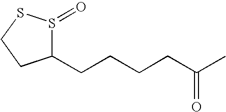 | 1-Nal | NH | 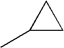 | | |
| 155 | 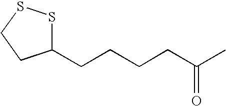 | 1-Nal | NH | CH₂COPh | | |
| 156 | 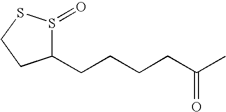 | 1-Nal | NH | CH₂COPh | | |
| 157 | 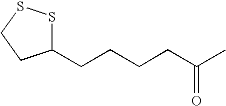 | 1-Nal | NH | 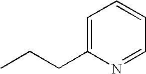 | | |
| 158 | 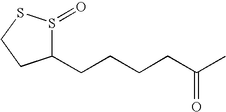 | 1-Nal | NH | 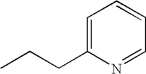 | | |
| 159 | 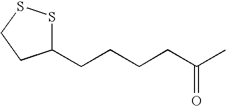 | 1-Nal | NH | 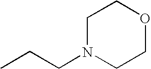 | | |
| 160 | 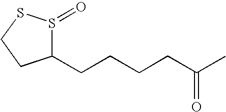 | 1-Nal | NH | 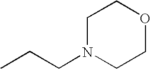 | | |
| 161 | 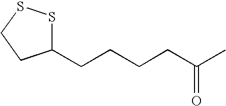 | 1-Nal | NH | CH₂CONH₂ | | |
| 162 | 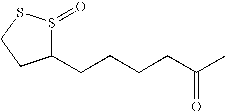 | 1-Nal | NH | CH₂CONH₂ | | |
| 163 | 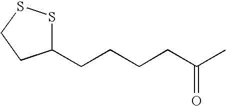 | 1-Nal | NH | CH₂COOEt | | |
| 164 | 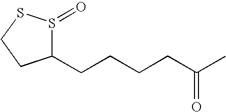 | 1-Nal | NH | CH₂COOEt | | |
| 165 | 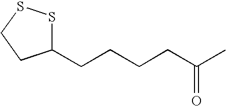 | 1-Nal | NH | CH₂COOH | | |
| 166 | 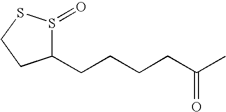 | 1-Nal | NH | CH₂COOH | | |
| 167 | 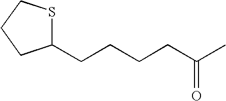 | 1-Nal | NH | Et | | |
| 168 | 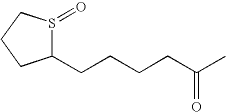 | 1-Nal | NH | Et | | |
| 169 | 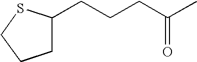 | 1-Nal | NH | Et | | |
| 170 | 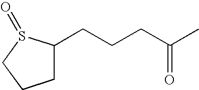 | 1-Nal | NH | Et | | |
| 171 | 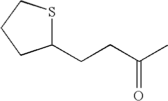 | 1-Nal | NH | Et | | |
| 172 | 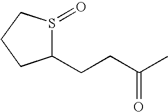 | 1-Nal | NH | Et | | |
| 173 | 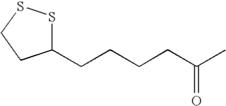 | 2-Nal | NH | Et | | |
| 174 | 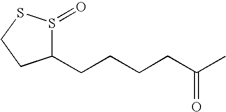 | 2-Nal | NH | Et | | |
| 175 | 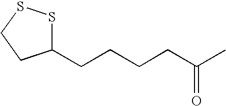 | 2-Nal | O | H | | |
| 176 | 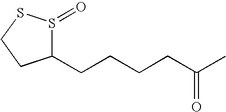 | 2-Nal | O | H | | |
| 177 | 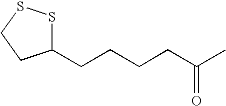 | 2-Nal | O | Me | | |
| 178 | 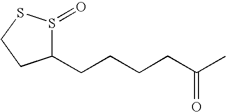 | 2-Nal | O | Me | | |
| 179 | 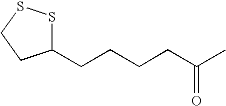 | 2-Nal | NH | 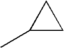 | | |
| 180 | 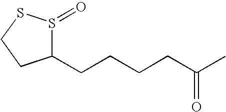 | 2-Nal | NH | 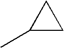 | | |
| 181 | 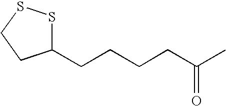 | 2-Nal | NH | CH₂COPh | | |
| 182 | 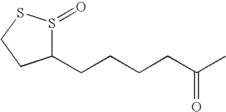 | 2-Nal | NH | CH₂COPh | | |
| 183 | 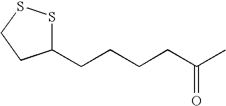 | 2-Nal | NH | 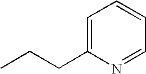 | | |
| 184 | 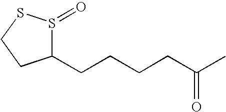 | 2-Nal | NH | 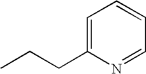 | | |
| 185 | 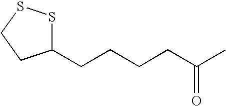 | 2-Nal | NH | 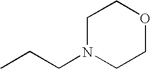 | | |
| 186 | 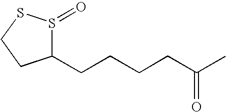 | 2-Nal | NH | 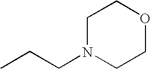 | | |
| 187 | 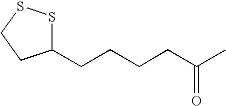 | 2-Nal | NH | CH₂CONH₂ | | |
| 188 | 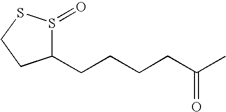 | 2-Nal | NH | CH₂CONH₂ | | |
| 189 | 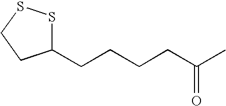 | 2-Nal | NH | CH₂COOEt | | |
| 190 | 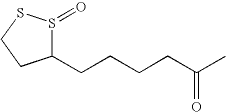 | 2-Nal | NH | CH₂COOEt | | |
| 191 | 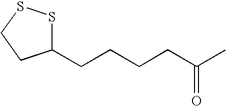 | 2-Nal | NH | CH₂COOH | | |
| 192 | 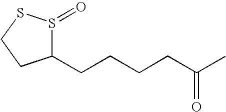 | 2-Nal | NH | CH₂COOH | | |
| 193 | 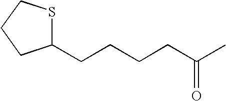 | 2-Nal | NH | Et | | |
| 194 | 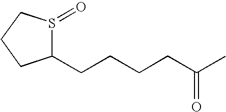 | 2-Nal | NH | Et | | |
| 195 | 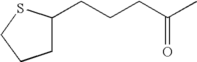 | 2-Nal | NH | Et | | |
| 196 | 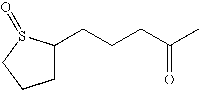 | 2-Nal | NH | Et | | |
| 197 | 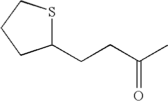 | 2-Nal | NH | Et | | |
| 198 | 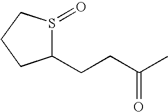 | 2-Nal | NH | Et | | |
| 199 | 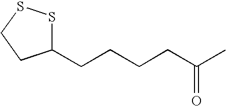 | Homophe | NH | Et | | |
| 200 | 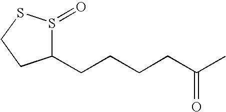 | Homophe | NH | Et | | |
| 201 | 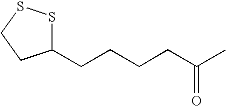 | Homophe | O | H | | |
| 202 | 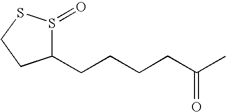 | Homophe | O | H | | |
| 203 | 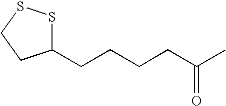 | Homophe | O | Me | | |
| 204 | 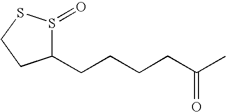 | Homophe | O | Me | | |
| 205 | 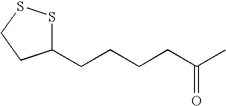 | Homophe | NH | 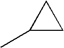 | | |
| 206 | 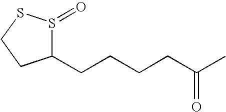 | Homophe | NH | 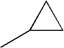 | | |
| 207 | 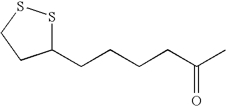 | Homophe | NH | CH₂COPh | | |
| 208 | 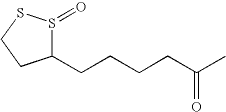 | Homophe | NH | CH₂COPh | | |
| 209 | 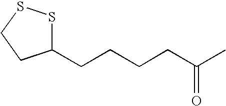 | Homophe | NH | 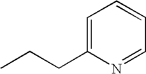 | | |
| 210 | 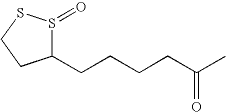 | Homophe | NH | 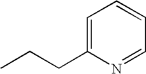 | | |
| 211 | 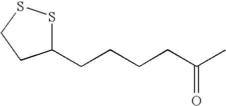 | Homophe | NH | 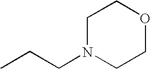 | | |
| 212 | 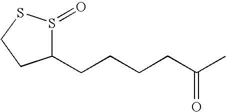 | Homophe | NH | 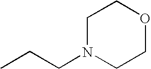 | | |
| 213 | 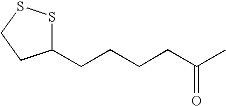 | Homophe | NH | CH₂CONH₂ | | |
| 214 | 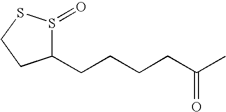 | Homophe | NH | CH₂CONH₂ | | |
| 215 | 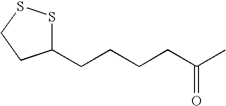 | Homophe | NH | CH₂COOEt | | |
| 216 | 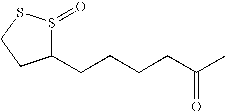 | Homophe | NH | CH₂COOEt | | |
| 217 | 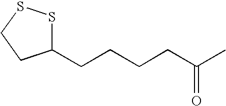 | Homophe | NH | CH₂COOH | | |
| 218 | 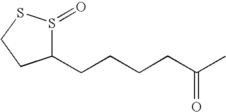 | Homophe | NH | CH₂COOH | | |
| 219 | 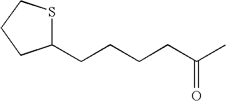 | Homophe | NH | Et | | |
| 220 | 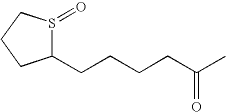 | Homophe | NH | Et | | |
| 221 | 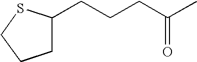 | Homophe | NH | Et | | |
| 222 | 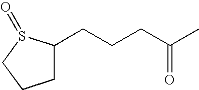 | Homophe | NH | Et | | |
| 223 | 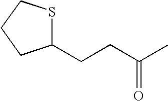 | Homophe | NH | Et | | |
| 224 | 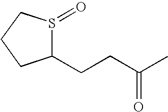 | Homophe | NH | Et | | |
| 225 | 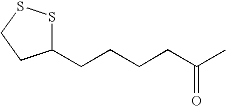 | Phe(4-F) | NH | Et | | |
| 226 | 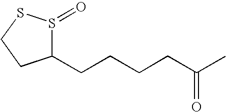 | Phe(4-F) | NH | Et | | |
| 227 | 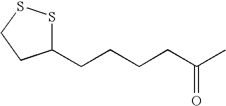 | Phe(4-Cl) | NH | Et | | |
| 228 | 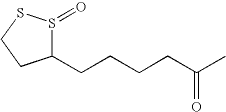 | Phe(4-Cl) | NH | Et | | |
| 229 | 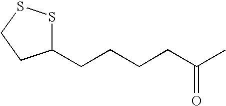 | Phe(3,4-Cl₂) | NH | Et | | |
| 230 | 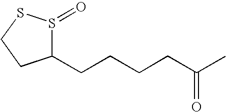 | Phe(3,4-Cl₂) | NH | Et | | |
| 231 | 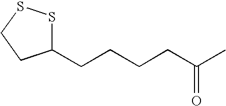 | Phe(4-OMe) | NH | Et | | |
| 232 | 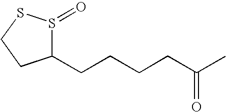 | Phe(4-OMe) | NH | Et | | |
| 233 | 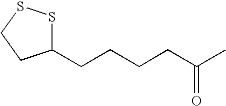 | 3-PyAla | NH | Et | | |
| 234 | 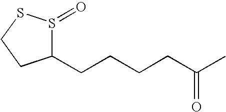 | 3-PyAla | NH | Et | | |
| 235 | 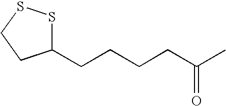 | 3-Benzo- thienylAla | NH | Et | | |
| 236 | 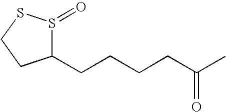 | 3-Benzo- thienylAla | NH | Et | | |
| 237 | 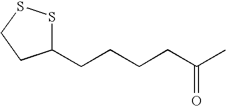 | CyclohexylAla | NH | Et | | |
| 238 | 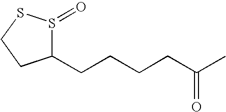 | CyclohexylAla | NH | Et | | |
| 239 | 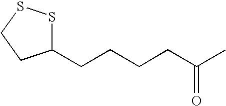 | Leu | NH | Et | | |
| 240 | 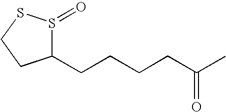 | Leu | NH | Et | | |

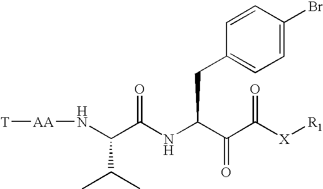

| | | | | | TLC | Mp. |
|---|---|---|---|---|---|---|
| Ex | T | AA | X | R₁ | [R_{f}(Solv.)] | [° C.] |
| 241 | 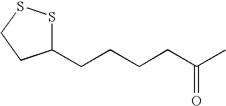 | Phe | NH | Et | 0.56 (CH₂Cl₂/MeOH 9:1) | 217-219 |
| 242 | 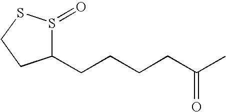 | Phe | NH | Et | | |
| 243 | 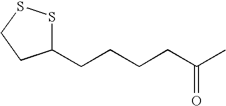 | Phe | O | H | | |
| 244 | 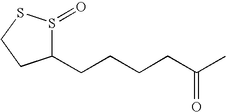 | Phe | O | H | | |
| 245 | 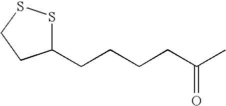 | Phe | O | Me | | |
| 246 | 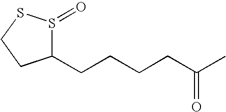 | Phe | O | Me | | |
| 247 | 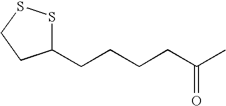 | Phe | NH | 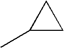 | | |
| 248 | 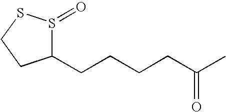 | Phe | NH | 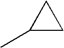 | | |
| 249 | 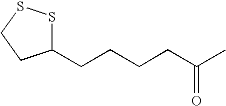 | Phe | NH | CH₂COPh | | |
| 250 | 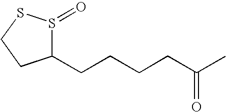 | Phe | NH | CH₂COPh | | |
| 251 | 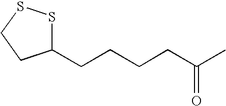 | Phe | NH | 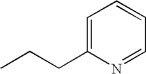 | | |
| 252 | 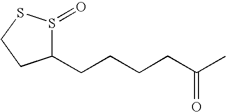 | Phe | NH | 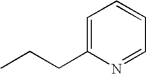 | | |
| 253 | 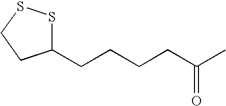 | Phe | NH | 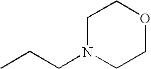 | | |
| 254 | 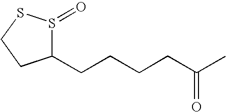 | Phe | NH | 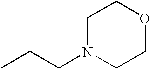 | | |
| 255 | 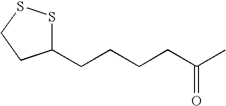 | Phe | NH | CH₂CONH₂ | | |
| 256 | 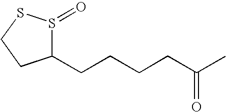 | Phe | NH | CH₂CONH₂ | | |
| 257 | 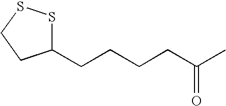 | Phe | NH | CH₂COOEt | | |
| 258 | 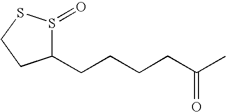 | Phe | NH | CH₂COOEt | | |
| 259 | 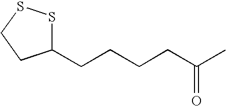 | Phe | NH | CH₂COOH | | |
| 260 | 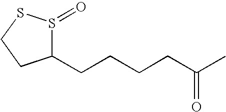 | Phe | NH | CH₂COOH | | |
| 261 | 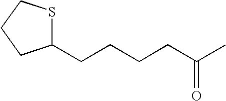 | Phe | NH | Et | | |
| 262 | 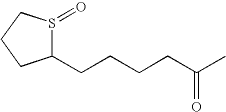 | Phe | NH | Et | | |
| 263 | 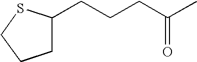 | Phe | NH | Et | | |
| 264 | 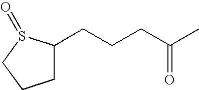 | Phe | NH | Et | | |
| 265 | 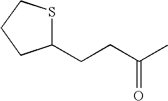 | Phe | NH | Et | | |
| 266 | 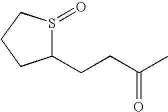 | Phe | NH | Et | | |
| 267 | 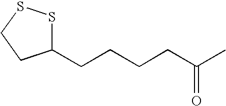 | 1-Nal | NH | Et | | |
| 268 | 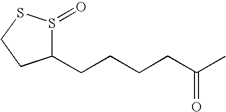 | 1-Nal | NH | Et | | |
| 269 | 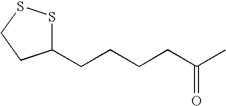 | 1-Nal | O | H | | |
| 270 | 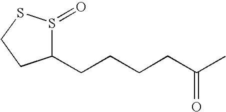 | 1-Nal | O | H | | |
| 271 | 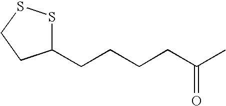 | 1-Nal | O | Me | | |
| 272 | 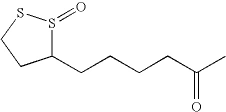 | 1-Nal | O | Me | | |
| 273 | 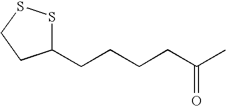 | 1-Nal | NH | 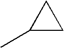 | | |
| 274 | 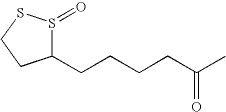 | 1-Nal | NH | 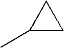 | | |
| 275 | 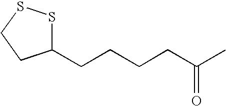 | 1-Nal | NH | CH₂COPh | | |
| 276 | 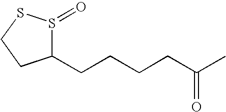 | 1-Nal | NH | CH₂COPh | | |
| 277 | 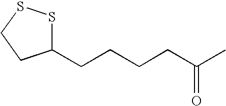 | 1-Nal | NH | 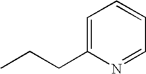 | | |
| 278 | 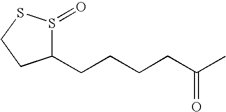 | 1-Nal | NH | 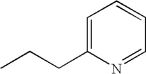 | | |
| 279 | 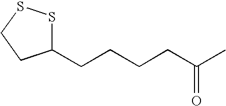 | 1-Nal | NH | 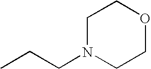 | | |
| 280 | 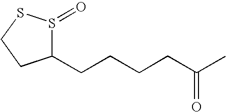 | 1-Nal | NH | 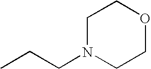 | | |
| 281 | 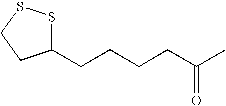 | 1-Nal | NH | CH₂CONH₂ | | |
| 282 | 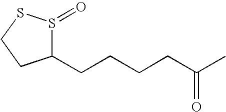 | 1-Nal | NH | CH₂CONH₂ | | |
| 283 | 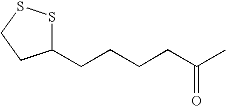 | 1-Nal | NH | CH₂COOEt | | |
| 284 | 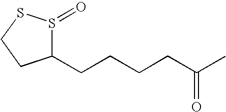 | 1-Nal | NH | CH₂COOEt | | |
| 285 | 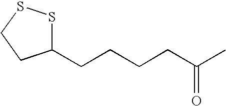 | 1-Nal | NH | CH₂COOH | | |
| 286 | 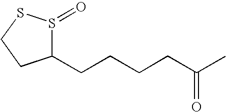 | 1-Nal | NH | CH₂COOH | | |
| 287 | 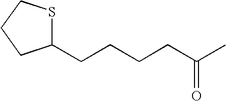 | 1-Nal | NH | Et | | |
| 288 | 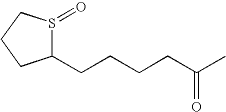 | 1-Nal | NH | Et | | |
| 289 | 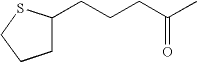 | 1-Nal | NH | Et | | |
| 290 | 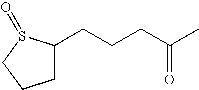 | 1-Nal | NH | Et | | |
| 291 | 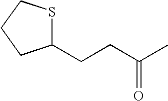 | 1-Nal | NH | Et | | |
| 292 | 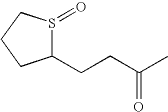 | 1-Nal | NH | Et | | |
| 293 | 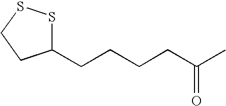 | 2-Nal | NH | Et | | |
| 294 | 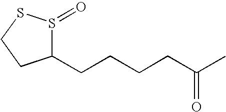 | 2-Nal | NH | Et | | |
| 295 | 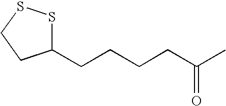 | 2-Nal | O | H | | |
| 296 | 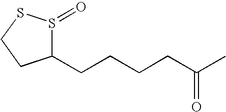 | 2-Nal | O | H | | |
| 297 | 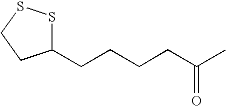 | 2-Nal | O | Me | | |
| 298 | 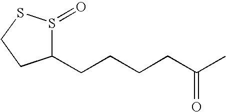 | 2-Nal | O | Me | | |
| 299 | 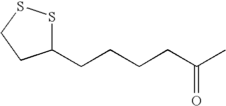 | 2-Nal | NH | 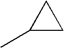 | | |
| 300 | 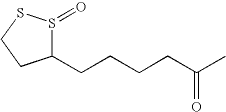 | 2-Nal | NH | 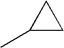 | | |
| 301 | 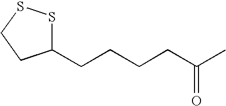 | 2-Nal | NH | CH₂COPh | | |
| 302 | 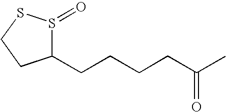 | 2-Nal | NH | CH₂COPh | | |
| 303 | 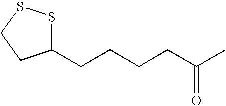 | 2-Nal | NH | 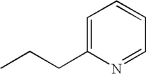 | | |
| 304 | 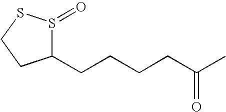 | 2-Nal | NH | 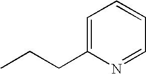 | | |
| 305 | 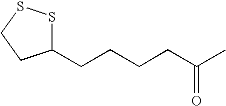 | 2-Nal | NH | 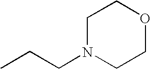 | | |
| 306 | 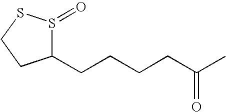 | 2-Nal | NH | 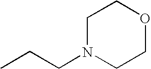 | | |
| 307 | 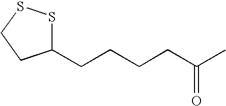 | 2-Nal | NH | CH₂CONH₂ | | |
| 308 | 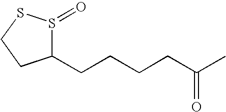 | 2-Nal | NH | CH₂CONH₂ | | |
| 309 | 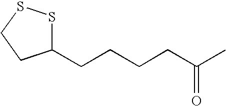 | 2-Nal | NH | CH₂COOEt | | |
| 310 | 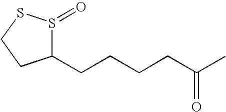 | 2-Nal | NH | CH₂COOEt | | |
| 311 | 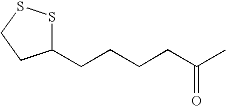 | 2-Nal | NH | CH₂COOH | | |
| 312 | 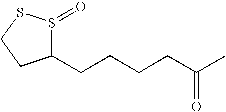 | 2-Nal | NH | CH₂COOH | | |
| 313 | 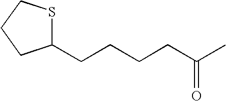 | 2-Nal | NH | Et | | |
| 314 | 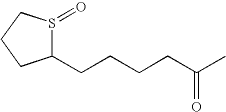 | 2-Nal | NH | Et | | |
| 315 | 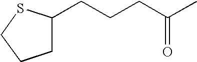 | 2-Nal | NH | Et | | |
| 316 | 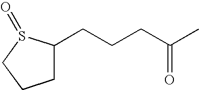 | 2-Nal | NH | Et | | |
| 317 | 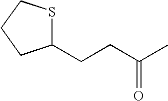 | 2-Nal | NH | Et | | |
| 318 | 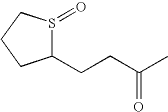 | 2-Nal | NH | Et | | |
| 319 | 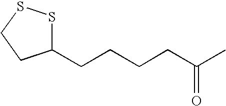 | Homophe | NH | Et | | |
| 320 | 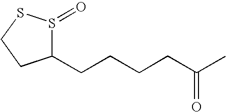 | Homophe | NH | Et | | |
| 321 | 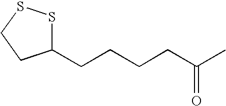 | Homophe | O | H | | |
| 322 | 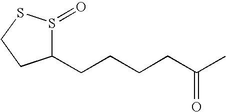 | Homophe | O | H | | |
| 323 | 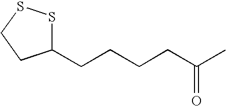 | Homophe | O | Me | | |
| 324 | 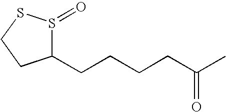 | Homophe | O | Me | | |
| 325 | 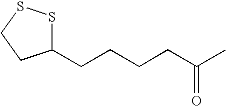 | Homophe | NH | 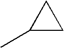 | | |
| 326 | 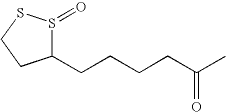 | Homophe | NH | 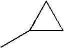 | | |
| 327 | 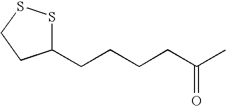 | Homophe | NH | CH₂COPh | | |
| 328 | 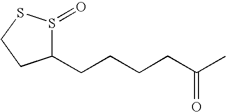 | Homophe | NH | CH₂COPh | | |
| 329 | 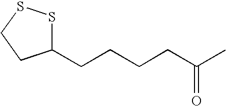 | Homophe | NH | 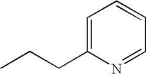 | | |
| 330 | 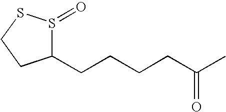 | Homophe | NH | 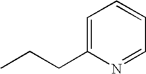 | | |
| 331 | 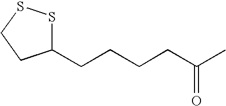 | Homophe | NH | 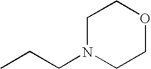 | | |
| 332 | 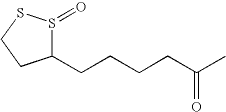 | Homophe | NH | 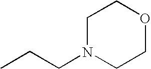 | | |
| 333 | 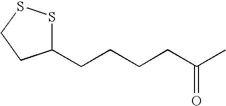 | Homophe | NH | CH₂CONH₂ | | |
| 334 | 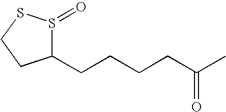 | Homophe | NH | CH₂CONH₂ | | |
| 335 | 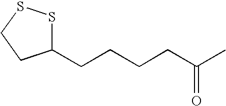 | Homophe | NH | CH₂COOEt | | |
| 336 | 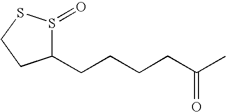 | Homophe | NH | CH₂COOEt | | |
| 337 | 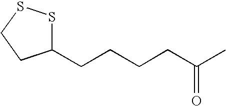 | Homophe | NH | CH₂COOH | | |
| 338 | 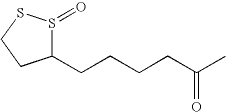 | Homophe | NH | CH₂COOH | | |
| 339 | 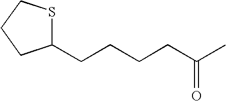 | Homophe | NH | Et | | |
| 340 | 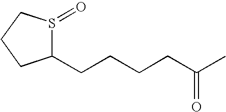 | Homophe | NH | Et | | |
| 341 | 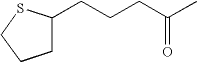 | Homophe | NH | Et | | |
| 342 | 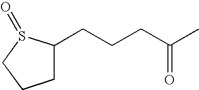 | Homophe | NH | Et | | |
| 343 | 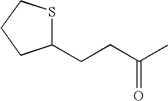 | Homophe | NH | Et | | |
| 344 | 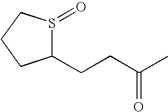 | Homophe | NH | Et | | |
| 345 | 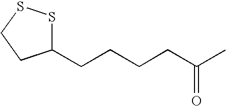 | Phe(4-F) | NH | Et | | |
| 346 | 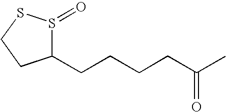 | Phe(4-F) | NH | Et | | |
| 347 | 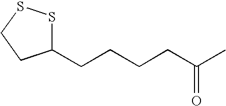 | Phe(4-Cl) | NH | Et | | |
| 348 | 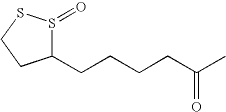 | Phe(4-Cl) | NH | Et | | |
| 349 | 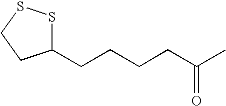 | Phe(3,4-Cl₂) | NH | Et | | |
| 350 | 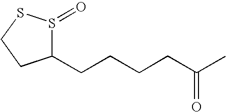 | Phe(3,4-Cl₂) | NH | Et | | |
| 351 | 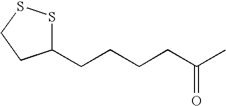 | Phe(4-OMe) | NH | Et | 0.61 (CH₂Cl₂/MeOH 9:1) | 218-220 |
| 352 | 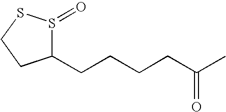 | Phe(4-OMe) | NH | Et | | |
| 353 | 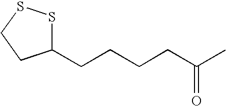 | 3-PyAla | NH | Et | | |
| 354 | 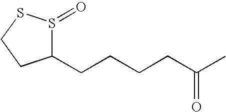 | 3-PyAla | NH | Et | | |
| 355 | 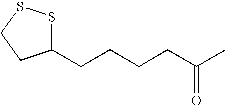 | 3-Benzo- thienylAla | NH | Et | | |
| 356 | 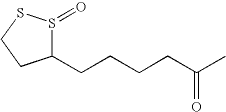 | 3-Benzo- thienylAla | NH | Et | | |
| 357 | 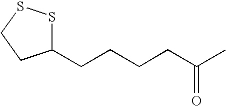 | CyclohexylAla | NH | Et | | |
| 358 | 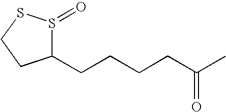 | CyclohexylAla | NH | Et | | |
| 359 | 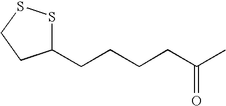 | Leu | NH | Et | | |
| 360 | 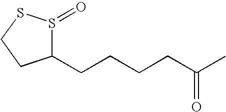 | Leu | NH | Et | | |

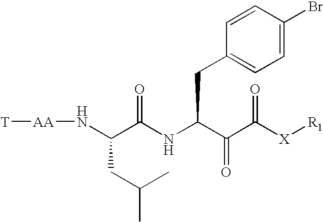

| | | | | | TLC | Mp. |
|---|---|---|---|---|---|---|
| Ex | T | AA | X | R₁ | [R_{f}(Solv.)] | [° C.] |
| 361 | 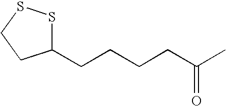 | Phe | NH | Et | 0.53 (CH₂Cl₂/MeOH 9:1) | 212-213 |
| 362 | 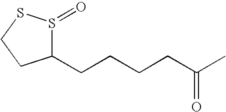 | Phe | NH | Et | | |
| 363 | 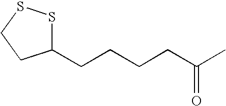 | Phe | O | H | | |
| 364 | 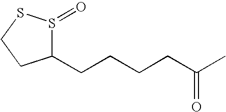 | Phe | O | H | | |
| 365 | 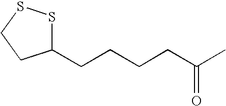 | Phe | O | Me | | |
| 366 | 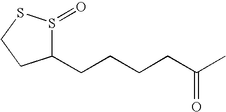 | Phe | O | Me | | |
| 367 | 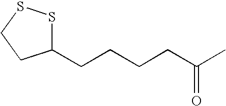 | Phe | NH | | | |
| 368 | 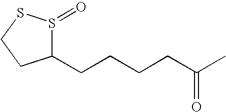 | Phe | NH | | | |
| 369 | 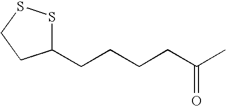 | Phe | NH | CH₂COPh | | |
| 370 | 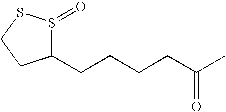 | Phe | NH | CH₂COPh | | |
| 371 | 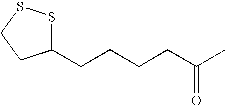 | Phe | NH | | | |
| 372 | 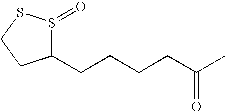 | Phe | NH | | | |
| 373 | 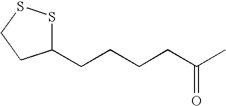 | Phe | NH | | | |
| 374 | 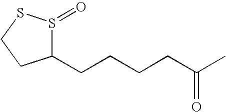 | Phe | NH | | | |
| 375 | 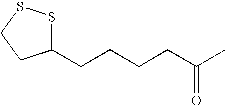 | Phe | NH | CH₂CONH₂ | | |
| 376 | 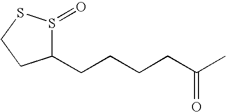 | Phe | NH | CH₂CONH₂ | | |
| 377 | 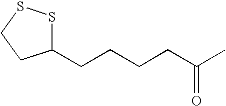 | Phe | NH | CH₂COOEt | 0.34 (CH₂Cl₂/MeOH 20:1) | 175 |
| 378 | 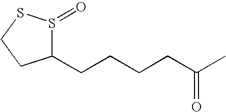 | Phe | NH | CH₂COOEt | | |
| 379 | 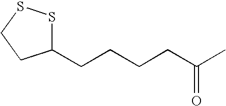 | Phe | NH | CH₂COOH | | |
| 380 | 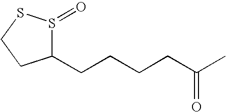 | Phe | NH | CH₂COOH | | |
| 381 | 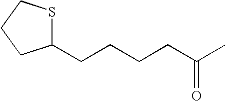 | Phe | NH | Et | | |
| 382 | 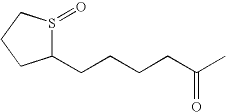 | Phe | NH | Et | | |
| 383 | 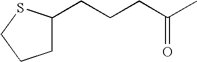 | Phe | NH | Et | | |
| 384 | 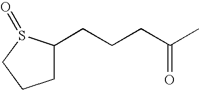 | Phe | NH | Et | | |
| 385 | 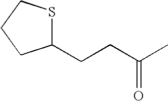 | Phe | NH | Et | | |
| 386 | 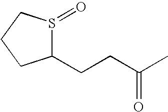 | Phe | NH | Et | | |
| 387 | 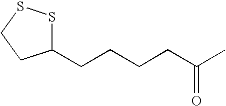 | 1-Nal | NH | Et | 0.54 (CH₂Cl₂/MeOH 9:1) | 194-196 |
| 388 | 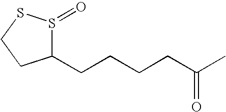 | 1-Nal | NH | Et | | |
| 389 | 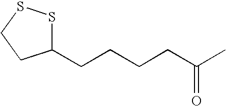 | 1-Nal | O | H | 0.00 (CH₂Cl₂/MeOH 95:5) | |
| 390 | 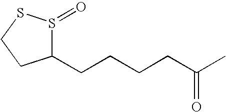 | 1-Nal | O | H | | |
| 391 | 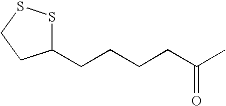 | 1-Nal | O | Me | | |
| 392 | 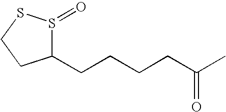 | 1-Nal | O | Me | | |
| 393 | 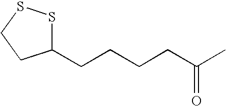 | 1-Nal | NH | | | |
| 394 | 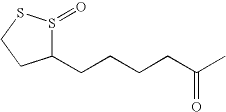 | 1-Nal | NH | | | |
| 395 | 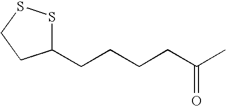 | 1-Nal | NH | CH₂COPh | | |
| 396 | 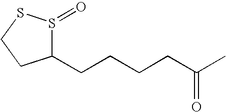 | 1-Nal | NH | CH₂COPh | | |
| 397 | 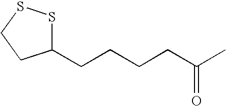 | 1-Nal | NH | | | |
| 398 | 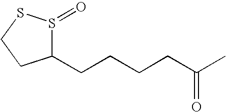 | 1-Nal | NH | | | |
| 399 | 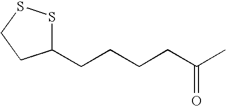 | 1-Nal | NH | | | |
| 400 | 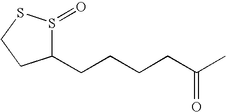 | 1-Nal | NH | | | |
| 401 | 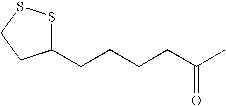 | 1-Nal | NH | CH₂CONH₂ | | |
| 402 | 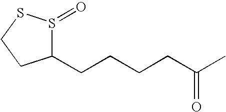 | 1-Nal | NH | CH₂CONH₂ | | |
| 403 | 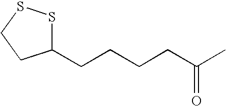 | 1-Nal | NH | CH₂COOEt | | |
| 404 | 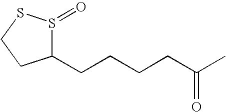 | 1-Nal | NH | CH₂COOEt | | |
| 405 | 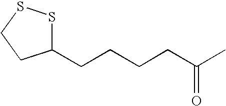 | 1-Nal | NH | CH₂COOH | | |
| 406 | 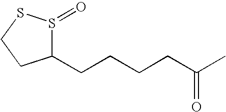 | 1-Nal | NH | CH₂COOH | | |
| 407 | 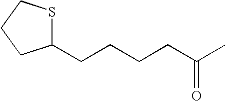 | 1-Nal | NH | Et | | |
| 408 | 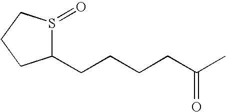 | 1-Nal | NH | Et | | |
| 409 | 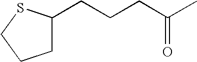 | 1-Nal | NH | Et | | |
| 410 | 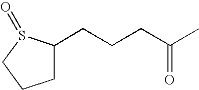 | 1-Nal | NH | Et | | |
| 411 | 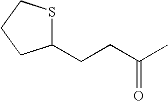 | 1-Nal | NH | Et | | |
| 412 | 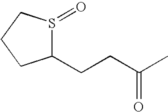 | 1-Nal | NH | Et | | |
| 413 | 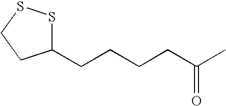 | 2-Nal | NH | Et | | |
| 414 | 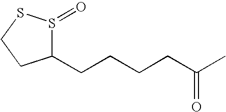 | 2-Nal | NH | Et | | |
| 415 | 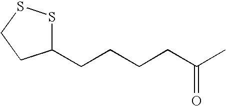 | 2-Nal | O | H | 0.00 (CH₂Cl₂/MeOH 95:5) | |
| 416 | 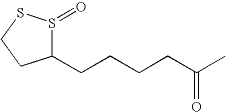 | 2-Nal | O | H | | |
| 417 | 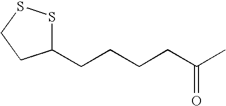 | 2-Nal | O | Me | | |
| 418 | 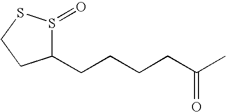 | 2-Nal | O | Me | | |
| 419 | 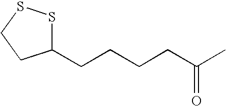 | 2-Nal | NH | | | |
| 420 | 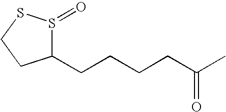 | 2-Nal | NH | | | |
| 421 | 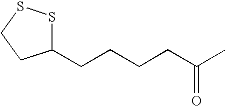 | 2-Nal | NH | CH₂COPh | | |
| 422 | 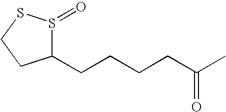 | 2-Nal | NH | CH₂COPh | | |
| 423 | 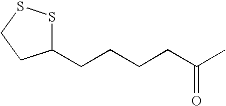 | 2-Nal | NH | | | |
| 424 | 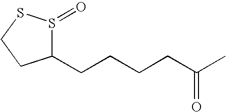 | 2-Nal | NH | | | |
| 425 | 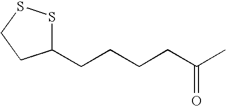 | 2-Nal | NH | | | |
| 426 | 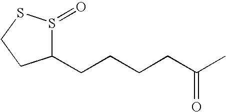 | 2-Nal | NH | | | |
| 427 | 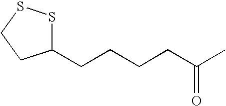 | 2-Nal | NH | CH₂CONH₂ | | |
| 428 | 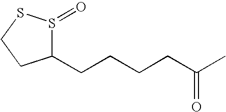 | 2-Nal | NH | CH₂CONH₂ | | |
| 429 | 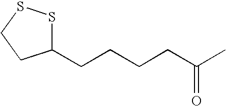 | 2-Nal | NH | CH₂COOEt | | |
| 430 | 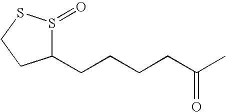 | 2-Nal | NH | CH₂COOEt | | |
| 431 | 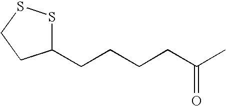 | 2-Nal | NH | CH₂COOH | | |
| 432 | 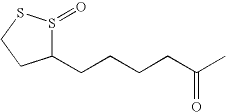 | 2-Nal | NH | CH₂COOH | | |
| 433 | 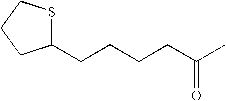 | 2-Nal | NH | Et | | |
| 434 | 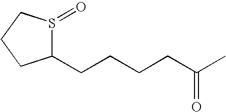 | 2-Nal | NH | Et | | |
| 435 | 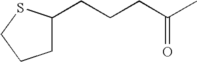 | 2-Nal | NH | Et | | |
| 436 | 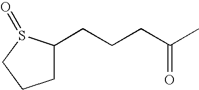 | 2-Nal | NH | Et | | |
| 437 | 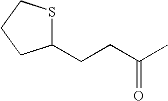 | 2-Nal | NH | Et | | |
| 438 | 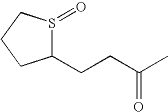 | 2-Nal | NH | Et | | |
| 439 | 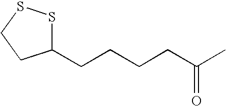 | Homophe | NH | Et | 0.65 (CH₂Cl₂/MeOH 9:1) | 206-207 |
| 440 | 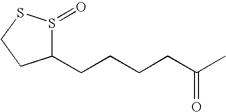 | Homophe | NH | Et | | |
| 441 | 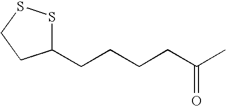 | Homophe | O | H | 0.00 (CH₂Cl₂/MeOH 95:5) | |
| 442 | 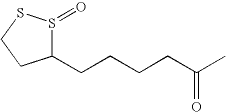 | Homophe | O | H | | |
| 443 | 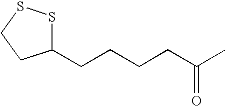 | Homophe | O | Me | | |
| 444 | 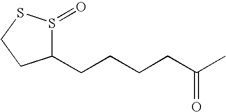 | Homophe | O | Me | | |
| 445 | 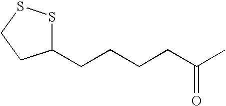 | Homophe | O | iPr | | |
| 446 | 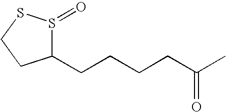 | Homophe | O | iPr | | |
| 447 | 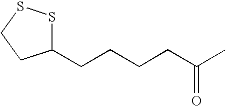 | Homophe | NH | | | |
| 448 | 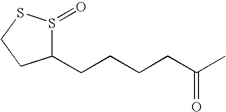 | Homophe | NH | | | |
| 449 | 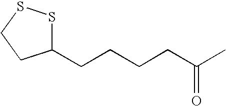 | Homophe | NH | CH₂COPh | | |
| 450 | 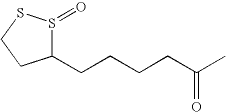 | Homophe | NH | CH₂COPh | | |
| 451 | 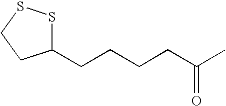 | Homophe | NH | | | |
| 452 | 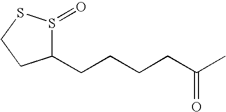 | Homophe | NH | | | |
| 453 | 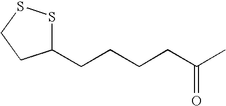 | Homophe | NH | | | |
| 454 | 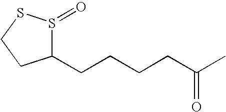 | Homophe | NH | | | |
| 455 | 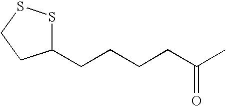 | Homophe | NH | CH₂CONH₂ | | |
| 456 | 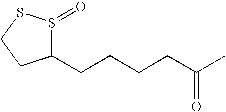 | Homophe | NH | CH₂CONH₂ | | |
| 457 | 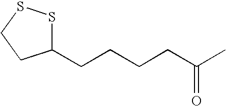 | Homophe | NH | CH₂COOEt | | |
| 458 | 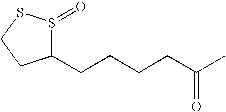 | Homophe | NH | CH₂COOEt | | |
| 459 | 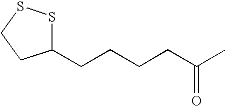 | Homophe | NH | CH₂COOH | | |
| 460 | 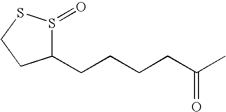 | Homophe | NH | CH₂COOH | | |
| 461 | 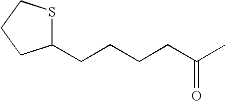 | Homophe | NH | Et | | |
| 462 | 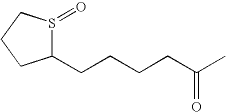 | Homophe | NH | Et | | |
| 463 | 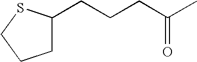 | Homophe | NH | Et | | |
| 464 | 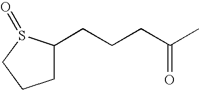 | Homophe | NH | Et | | |
| 465 | 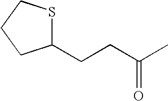 | Homophe | NH | Et | | |
| 466 | 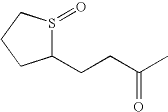 | Homophe | NH | Et | | |
| 467 | 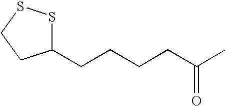 | Phe(4-F) | NH | Et | 0.57 (CH₂Cl₂/MeOH 9:1) | 220-221 |
| 468 | 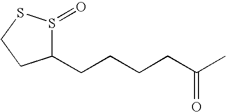 | Phe(4-F) | NH | Et | | |
| 469 | 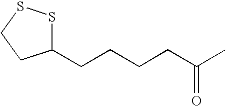 | Phe(4-Cl) | NH | Et | 0.50 (CH₂Cl₂/MeOH 9:1) | 219-220 |
| 470 | 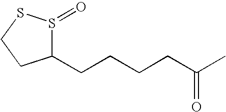 | Phe(4-Cl) | NH | Et | | |
| 471 | 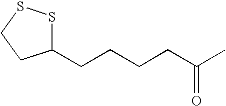 | Phe(4-Cl) | NH | CH₂COOEt | 0.55 (CH₂Cl₂/MeOH 10:1) | 181 |
| 472 | 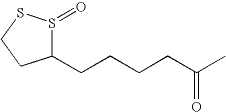 | Phe(4-Cl) | NH | CH₂COOEt | | |
| 473 | 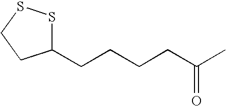 | Phe(3,4-Cl₂) | NH | Et | 0.48 (CH₂Cl₂/MeOH 9:1) | 222-223 |
| 474 | 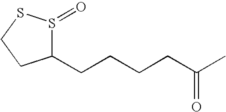 | Phe(3,4-Cl₂) | NH | Et | | |
| 475 | 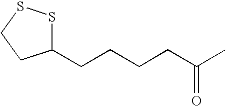 | Phe(4-OMe) | NH | Et | 0.55 (CH₂Cl₂/MeOH 9:1) | 196-198 |
| 476 | 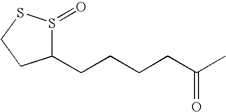 | Phe(4-OMe) | NH | Et | | |
| 477 | 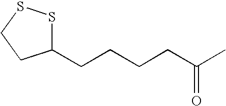 | 3-PyAla | NH | Et | | |
| 478 | 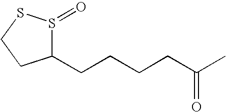 | 3-PyAla | NH | Et | | |
| 479 | 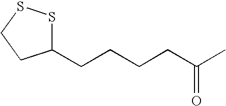 | 3-PyAla | NH | CH₂COOEt | 0.43 (CH₂Cl₂/MeOH 10:1) | 162 |
| 480 | 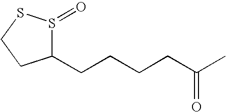 | 3-PyAla | NH | CH₂COOEt | 0.40 (CH₂Cl₂/MeOH 10:1) | |
| 481 | 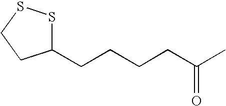 | 3-Benzo- thienylAla | NH | Et | | |
| 482 | 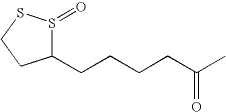 | 3-Benzo- thienylAla | NH | Et | | |
| 483 | 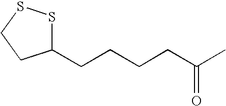 | CyclohexylAla | NH | Et | | |
| 484 | 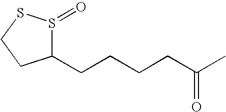 | CyclohexylAla | NH | Et | | |
| 485 | 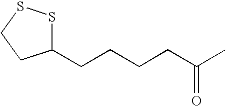 | Leu | NH | Et | 0.53 (CH₂Cl₂/MeOH 9:1) | 199-200 |
| 486 | 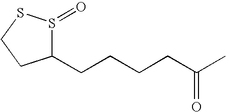 | Leu | NH | Et | | |

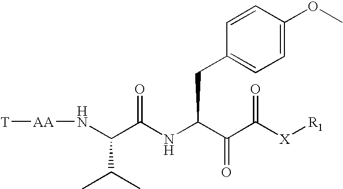

| | | | | | TLC | Mp. |
|---|---|---|---|---|---|---|
| Ex | T | AA | X | R₁ | [R_{f}(Solv.)] | [° C.] |
| 487 | 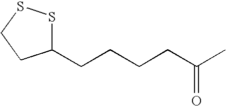 | Phe | NH | Et | | |
| 488 | 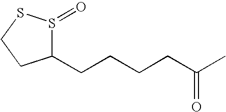 | Phe | NH | Et | | |
| 489 | 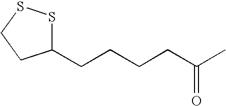 | Phe | O | H | | |
| 490 | 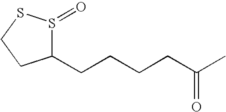 | Phe | O | H | | |
| 491 | 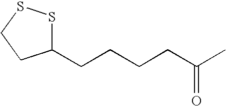 | Phe | O | Me | | |
| 492 | 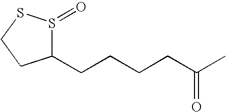 | Phe | O | Me | | |
| 493 | 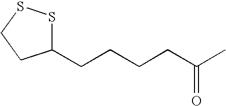 | Phe | NH | 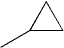 | | |
| 494 | 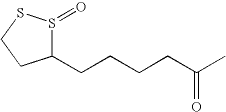 | Phe | NH | 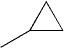 | | |
| 495 | 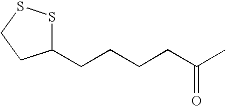 | Phe | NH | CH₂COPh | | |
| 496 | 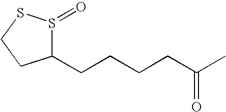 | Phe | NH | CH₂COPh | | |
| 497 | 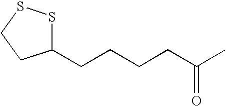 | Phe | NH | 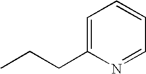 | | |
| 498 | 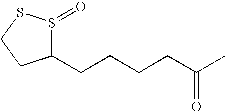 | Phe | NH | 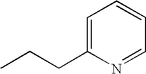 | | |
| 499 | 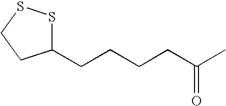 | Phe | NH | 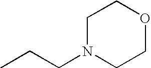 | | |
| 500 | 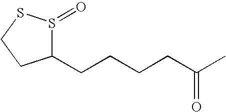 | Phe | NH | 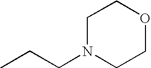 | | |
| 501 | 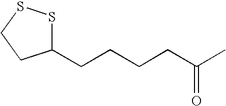 | Phe | NH | CH₂CONH₂ | | |
| 502 | 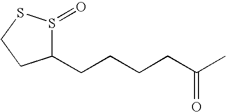 | Phe | NH | CH₂CONH₂ | | |
| 503 | 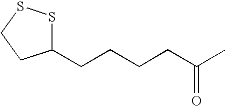 | Phe | NH | CH₂COOEt | | |
| 504 | 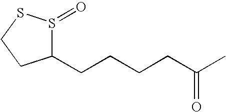 | Phe | NH | CH₂COOEt | | |
| 505 | 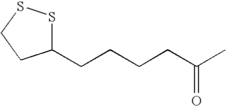 | Phe | NH | CH₂COOH | | |
| 506 | 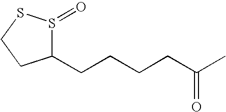 | Phe | NH | CH₂COOH | | |
| 507 | 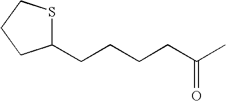 | Phe | NH | Et | | |
| 508 | 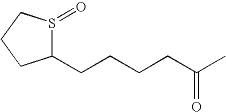 | Phe | NH | Et | | |
| 509 | 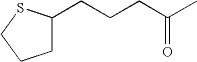 | Phe | NH | Et | | |
| 510 | 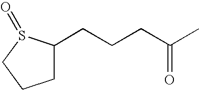 | Phe | NH | Et | | |
| 511 | 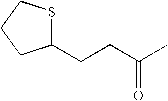 | Phe | NH | Et | | |
| 512 | 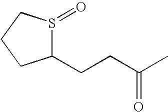 | Phe | NH | Et | | |
| 513 | 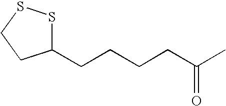 | 1-Nal | NH | Et | 0.47 (CH₂Cl₂/MeOH 9:1) | 203-205 |
| 514 | 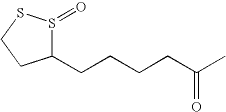 | 1-Nal | NH | Et | | |
| 515 | 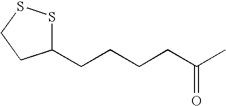 | 1-Nal | O | H | | |
| 516 | 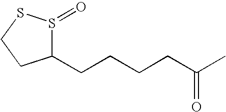 | 1-Nal | O | H | 0.67/0.73 (CH₂Cl₂/MeOH/ AcOH 5:1:0.1) | 208-210 |
| 517 | 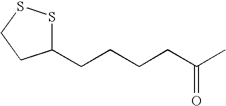 | 1-Nal | O | Me | | |
| 518 | 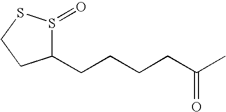 | 1-Nal | O | Me | 0.39 (CH₂Cl₂/MeOH 95:5) | 212-213 |
| 519 | 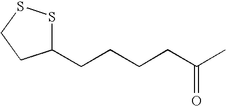 | 1-Nal | NH | 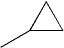 | | |
| 520 | 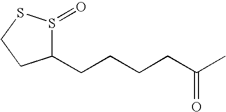 | 1-Nal | NH | 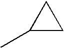 | | |
| 521 | 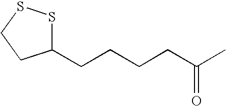 | 1-Nal | NH | CH₂COPh | | |
| 522 | 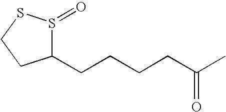 | 1-Nal | NH | CH₂COPh | | |
| 523 | 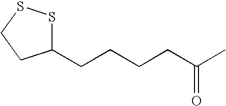 | 1-Nal | NH | 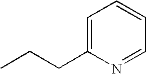 | | |
| 524 | 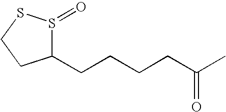 | 1-Nal | NH | 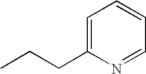 | | |
| 525 | 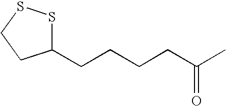 | 1-Nal | NH | 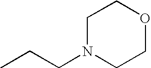 | | |
| 526 | 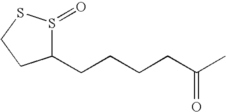 | 1-Nal | NH | 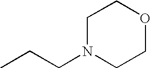 | | |
| 527 | 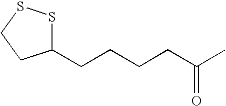 | 1-Nal | NH | CH₂CONH₂ | | |
| 528 | 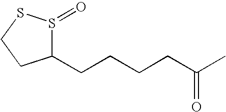 | 1-Nal | NH | CH₂CONH₂ | | |
| 529 | 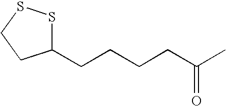 | 1-Nal | NH | CH₂COOEt | | |
| 530 | 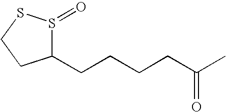 | 1-Nal | NH | CH₂COOEt | | |
| 531 | 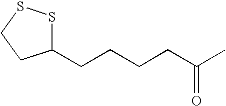 | 1-Nal | NH | CH₂COOH | | |
| 532 | 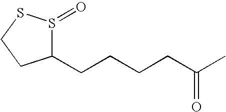 | 1-Nal | NH | CH₂COOH | | |
| 533 | 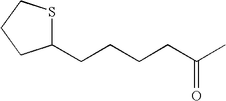 | 1-Nal | NH | Et | | |
| 534 | 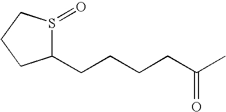 | 1-Nal | NH | Et | | |
| 535 | 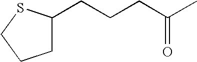 | 1-Nal | NH | Et | | |
| 536 | 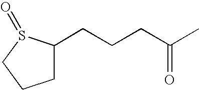 | 1-Nal | NH | Et | | |
| 537 | 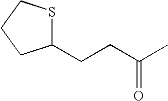 | 1-Nal | NH | Et | | |
| 538 | 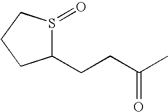 | 1-Nal | NH | Et | | |
| 539 | 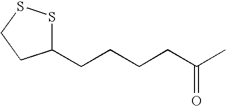 | 2-Nal | NH | Et | 0.52 (CH₂Cl₂/MeOH 9:1) | 212-213 |
| 540 | 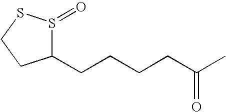 | 2-Nal | NH | Et | 0.46 (CH₂Cl₂/MeOH 9:1) | 196-198 |
| 541 | 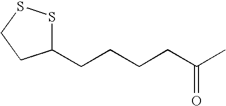 | D-2-Nal | NH | Et | 0.51 (CH₂Cl₂/MeOH 9:1) | 225-227 |
| 542 | 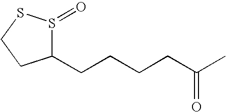 | D-2-Nal | NH | Et | 0.42 (CH₂Cl₂/MeOH 9:1) | 196-198 |
| 543 | 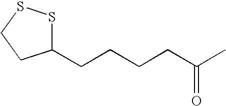 | 2-Nal | O | H | | |
| 544 | 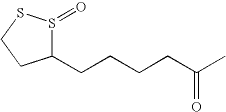 | 2-Nal | O | H | | |
| 545 | 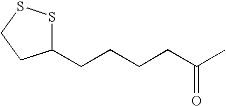 | 2-Nal | O | Me | | |
| 546 | 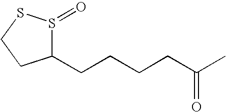 | 2-Nal | O | Me | | |
| 547 | 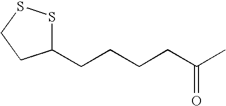 | 2-Nal | NH | 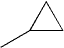 | | |
| 548 | 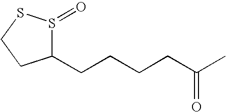 | 2-Nal | NH | 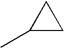 | | |
| 549 | 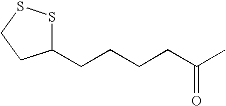 | 2-Nal | NH | CH₂COPh | | |
| 550 | 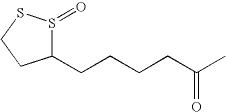 | 2-Nal | NH | CH₂COPh | | |
| 551 | 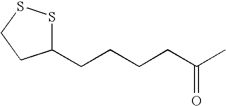 | 2-Nal | NH | 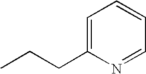 | | |
| 552 | 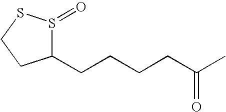 | 2-Nal | NH | 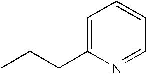 | | |
| 553 | 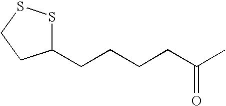 | 2-Nal | NH | 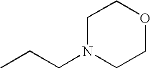 | | |
| 554 | 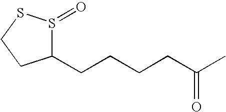 | 2-Nal | NH | 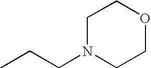 | | |
| 555 | 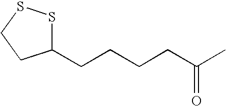 | 2-Nal | NH | CH₂CONH₂ | | |
| 556 | 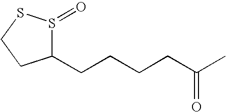 | 2-Nal | NH | CH₂CONH₂ | | |
| 557 | 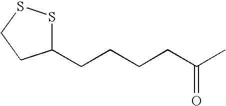 | 2-Nal | NH | CH₂COOEt | | |
| 558 | 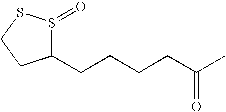 | 2-Nal | NH | CH₂COOEt | | |
| 559 | 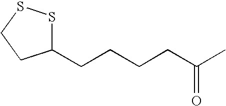 | 2-Nal | NH | CH₂COOH | | |
| 560 | 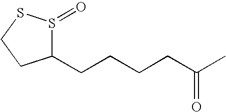 | 2-Nal | NH | CH₂COOH | | |
| 561 | 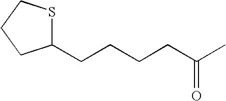 | 2-Nal | NH | Et | | |
| 562 | 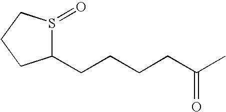 | 2-Nal | NH | Et | | |
| 563 | 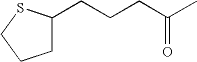 | 2-Nal | NH | Et | | |
| 564 | 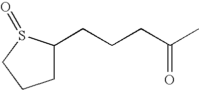 | 2-Nal | NH | Et | | |
| 565 | 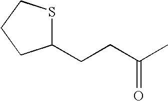 | 2-Nal | NH | Et | | |
| 566 | 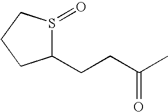 | 2-Nal | NH | Et | | |
| 567 | 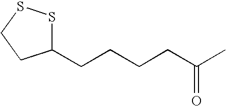 | Homophe | NH | Et | 0.51 (CH₂Cl₂/MeOH 9:1) | 208-211 |
| 568 | 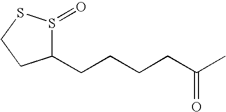 | Homophe | NH | Et | | |
| 569 | 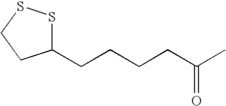 | Homophe | O | H | | |
| 570 | 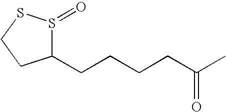 | Homophe | O | H | | |
| 571 | 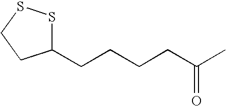 | Homophe | O | Me | | |
| 572 | 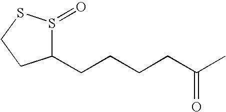 | Homophe | O | Me | | |
| 573 | 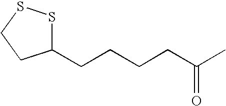 | Homophe | NH | 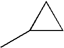 | | |
| 574 | 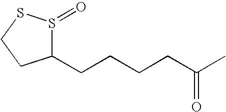 | Homophe | NH | 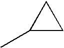 | | |
| 575 | 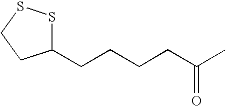 | Homophe | NH | CH₂COPh | | |
| 576 | 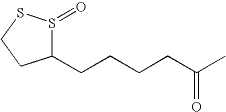 | Homophe | NH | CH₂COPh | | |
| 577 | 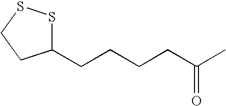 | Homophe | NH | 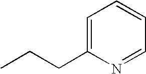 | | |
| 578 | 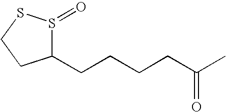 | Homophe | NH | 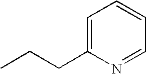 | | |
| 579 | 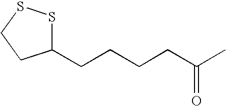 | Homophe | NH | 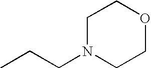 | | |
| 580 | 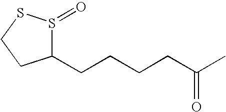 | Homophe | NH | 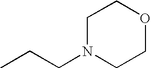 | | |
| 581 | 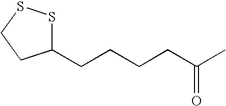 | Homophe | NH | CH₂CONH₂ | | |
| 582 | 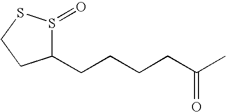 | Homophe | NH | CH₂CONH₂ | | |
| 583 | 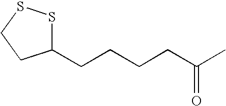 | Homophe | NH | CH₂COOEt | | |
| 584 | 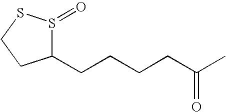 | Homophe | NH | CH₂COOEt | | |
| 585 | 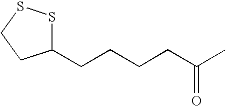 | Homophe | NH | CH₂COOH | | |
| 586 | 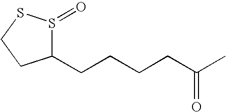 | Homophe | NH | CH₂COOH | | |
| 587 | 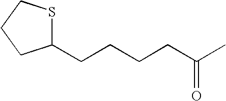 | Homophe | NH | Et | | |
| 588 | 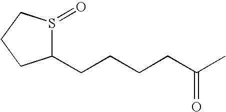 | Homophe | NH | Et | | |
| 589 | 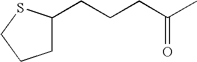 | Homophe | NH | Et | | |
| 590 | 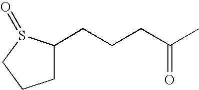 | Homophe | NH | Et | | |
| 591 | 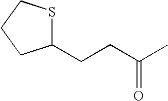 | Homophe | NH | Et | | |
| 592 | 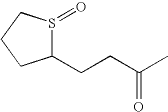 | Homophe | NH | Et | | |
| 593 | 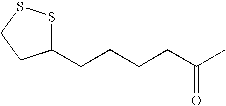 | StyrylAla | NH | Et | 0.53 (CH₂Cl₂/MeOH 20:1) | 221 |
| 594 | 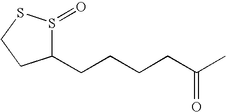 | StyrylAla | NH | Et | | |
| 595 | 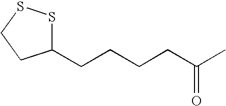 | Phe(4-F) | NH | Et | | |
| 596 | 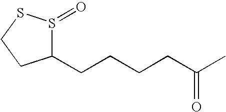 | Phe(4-F) | NH | Et | | |
| 597 | 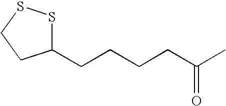 | Phe(4-Cl) | NH | Et | | |
| 598 | 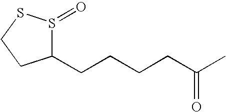 | Phe(4-Cl) | NH | Et | | |
| 599 | 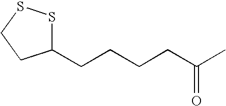 | Phe(3,4-Cl₂) | NH | Et | | |
| 600 | 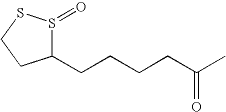 | Phe(3,4-Cl₂) | NH | Et | | |
| 601 | 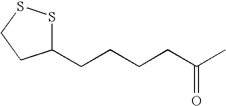 | Phe(4-OMe) | NH | Et | | |
| 602 | 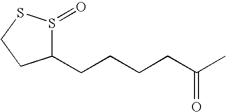 | Phe(4-OMe) | NH | Et | | |
| 603 | 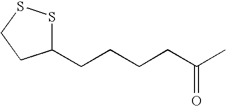 | 3-PyAla | NH | Et | | |
| 604 | 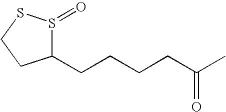 | 3-PyAla | NH | Et | | |
| 605 | 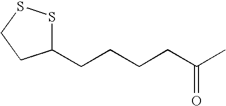 | 3-Benzo- thienylAla | NH | Et | 0.60 (CH₂Cl₂/MeOH 9:1) | 206-207 |
| 606 | 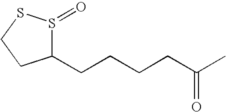 | 3-Benzo- thienylAla | NH | Et | 0.45 (CH₂Cl₂/MeOH 9:1) | 190-192 |
| 607 | 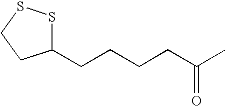 | CyclohexylAla | NH | Et | 0.44 (CH₂Cl₂/MeOH 20:1) | 190 |
| 608 | 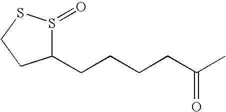 | CyclohexylAla | NH | Et | | |
| 609 | 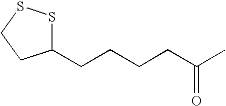 | Leu | NH | Et | | |
| 610 | 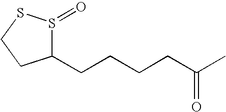 | Leu | NH | Et | | |

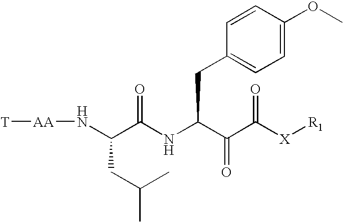

| | | | | | TLC | Mp. |
|---|---|---|---|---|---|---|
| Ex | T | AA | X | R₁ | [R_{f}(Solv.)] | [° C.] |
| 611 | 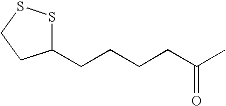 | Phe | NH | Et | | |
| 612 | 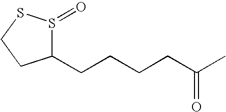 | Phe | NH | Et | | |
| 613 | 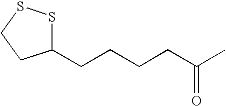 | Phe | O | H | | |
| 614 | 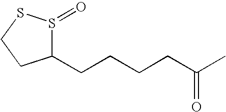 | Phe | O | H | | |
| 615 | 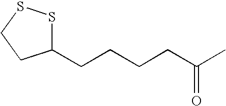 | Phe | O | Me | | |
| 616 | 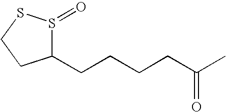 | Phe | O | Me | | |
| 617 | 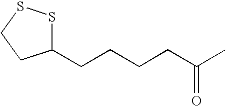 | Phe | NH | 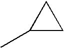 | | |
| 618 | 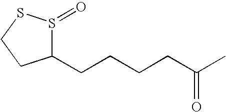 | Phe | NH | 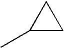 | | |
| 619 | 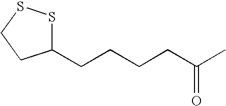 | Phe | NH | CH₂COPh | | |
| 620 | 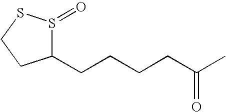 | Phe | NH | CH₂COPh | | |
| 621 | 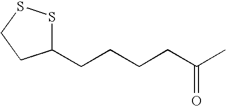 | Phe | NH | 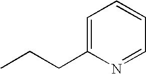 | | |
| 622 | 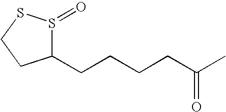 | Phe | NH | 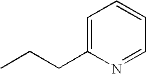 | | |
| 623 | 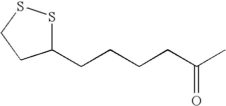 | Phe | NH | 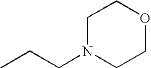 | | |
| 624 | 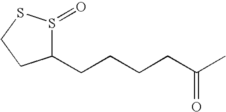 | Phe | NH | 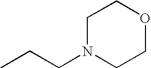 | | |
| 625 | 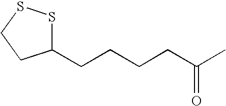 | Phe | NH | CH₂CONH₂ | | |
| 626 | 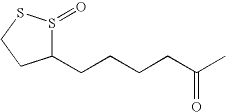 | Phe | NH | CH₂CONH₂ | | |
| 627 | 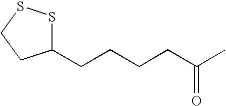 | Phe | NH | CH₂COOEt | | |
| 628 | 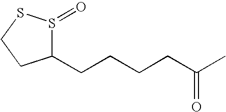 | Phe | NH | CH₂COOEt | | |
| 629 | 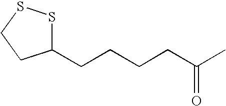 | Phe | NH | CH₂COOH | | |
| 630 | 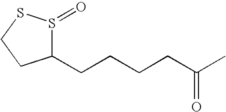 | Phe | NH | CH₂COOH | | |
| 631 | 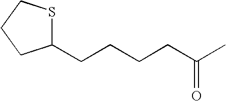 | Phe | NH | Et | | |
| 632 | 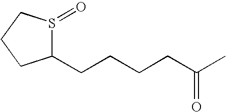 | Phe | NH | Et | | |
| 633 | 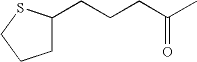 | Phe | NH | Et | | |
| 634 | 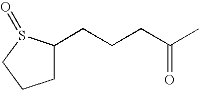 | Phe | NH | Et | | |
| 635 | 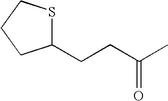 | Phe | NH | Et | | |
| 636 | 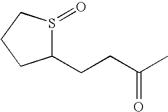 | Phe | NH | Et | | |
| 637 | 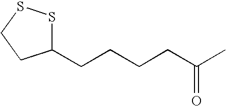 | 1-Nal | NH | Et | 0.47 (CH₂Cl₂/MeOH 9:1) | 188-190 |
| 638 | 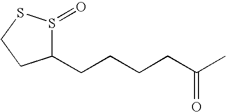 | 1-Nal | NH | Et | 0.43 (CH₂Cl₂/MeOH 9:1) | 166-167 |
| 639 | 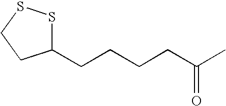 | D-1-Nal | NH | Et | 0.54 (CH₂Cl₂/MeOH 9:1) | 184-187 |
| 640 | 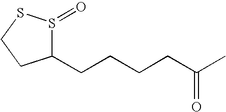 | D-1-Nal | NH | Et | 0.46 (CH₂Cl₂/MeOH 9:1) | 163-164 |
| 641 | 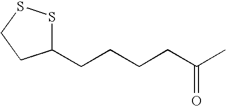 | 1-Nal | O | H | | |
| 642 | 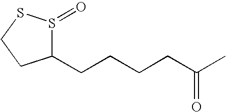 | 1-Nal | O | H | | |
| 643 | 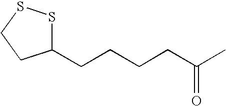 | 1-Nal | O | Me | | |
| 644 | 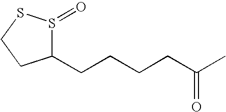 | 1-Nal | O | Me | | |
| 645 | 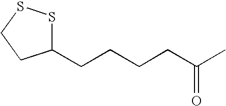 | 1-Nal | NH | 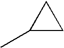 | | |
| 646 | 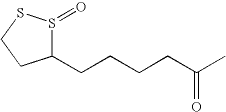 | 1-Nal | NH | 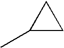 | | |
| 647 | 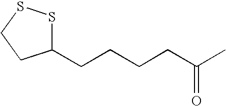 | 1-Nal | NH | CH₂COPh | | |
| 648 | 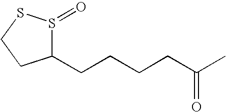 | 1-Nal | NH | CH₂COPh | | |
| 649 | 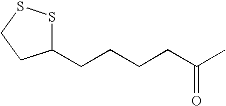 | 1-Nal | NH | 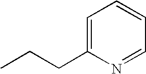 | | |
| 650 | 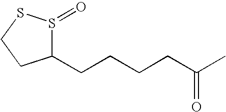 | 1-Nal | NH | 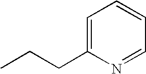 | | |
| 651 | 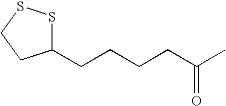 | 1-Nal | NH | 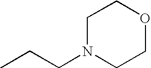 | | |
| 652 | 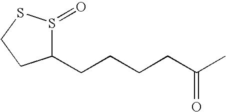 | 1-Nal | NH | 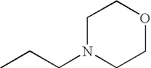 | | |
| 653 | 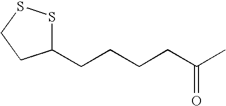 | 1-Nal | NH | CH₂CONH₂ | | |
| 654 | 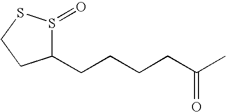 | 1-Nal | NH | CH₂CONH₂ | | |
| 655 | 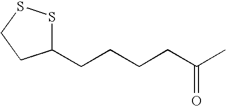 | 1-Nal | NH | CH₂COOEt | | |
| 656 | 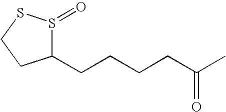 | 1-Nal | NH | CH₂COOEt | | |
| 657 | 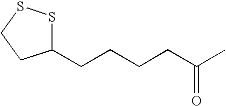 | 1-Nal | NH | CH₂COOH | | |
| 658 | 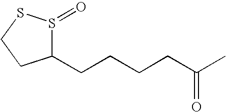 | 1-Nal | NH | CH₂COOH | | |
| 659 | 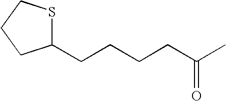 | 1-Nal | NH | Et | | |
| 660 | 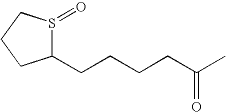 | 1-Nal | NH | Et | | |
| 661 | 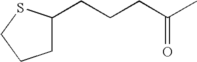 | 1-Nal | NH | Et | | |
| 662 | 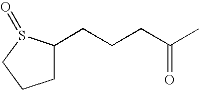 | 1-Nal | NH | Et | | |
| 663 | 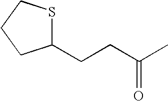 | 1-Nal | NH | Et | | |
| 664 | 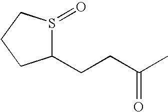 | 1-Nal | NH | Et | | |
| 665 | 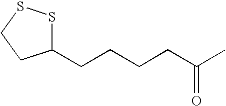 | 2-Nal | NH | Et | | |
| 666 | 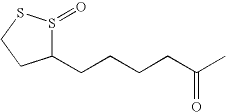 | 2-Nal | NH | Et | | |
| 667 | 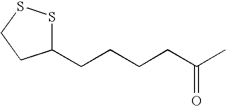 | 2-Nal | O | H | | |
| 668 | 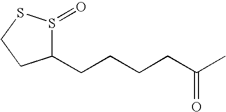 | 2-Nal | O | H | | |
| 669 | 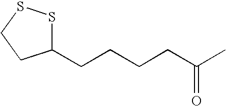 | 2-Nal | O | Me | | |
| 670 | 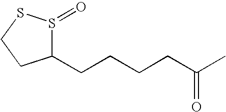 | 2-Nal | O | Me | | |
| 671 | 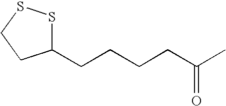 | 2-Nal | NH | 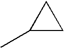 | | |
| 672 | 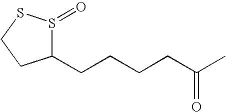 | 2-Nal | NH | 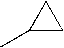 | | |
| 673 | 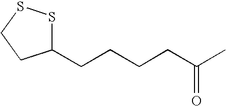 | 2-Nal | NH | CH₂COPh | | |
| 674 | 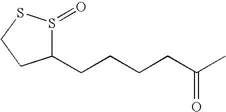 | 2-Nal | NH | CH₂COPh | | |
| 675 | 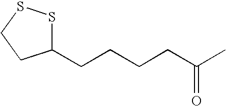 | 2-Nal | NH | 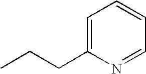 | | |
| 676 | 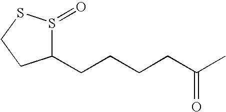 | 2-Nal | NH | 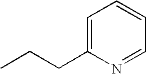 | | |
| 677 | 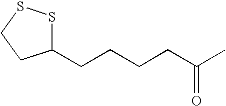 | 2-Nal | NH | 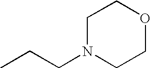 | | |
| 678 | 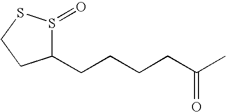 | 2-Nal | NH | 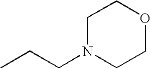 | | |
| 679 | 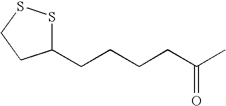 | 2-Nal | NH | CH₂CONH₂ | | |
| 680 | 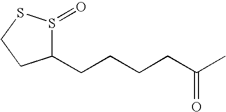 | 2-Nal | NH | CH₂CONH₂ | | |
| 681 | 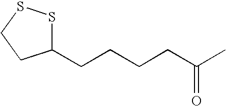 | 2-Nal | NH | CH₂COOEt | | |
| 682 | 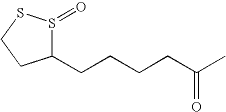 | 2-Nal | NH | CH₂COOEt | | |
| 683 | 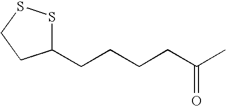 | 2-Nal | NH | CH₂COOH | | |
| 684 | 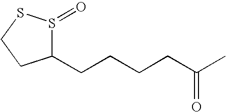 | 2-Nal | NH | CH₂COOH | | |
| 685 | 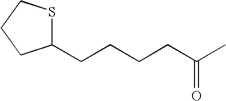 | 2-Nal | NH | Et | | |
| 686 | 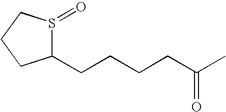 | 2-Nal | NH | Et | | |
| 687 | 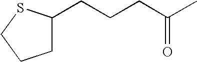 | 2-Nal | NH | Et | | |
| 688 | 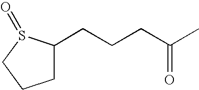 | 2-Nal | NH | Et | | |
| 689 | 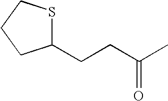 | 2-Nal | NH | Et | | |
| 690 | 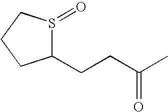 | 2-Nal | NH | Et | | |
| 691 | 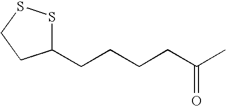 | Homophe | NH | Et | | |
| 692 | 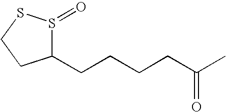 | Homophe | NH | Et | | |
| 693 | 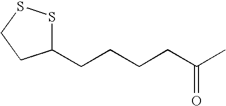 | Homophe | O | H | | |
| 694 | 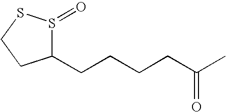 | Homophe | O | H | | |
| 695 | 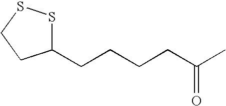 | Homophe | O | Me | | |
| 696 | 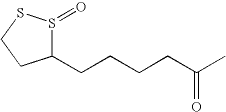 | Homophe | O | Me | | |
| 697 | 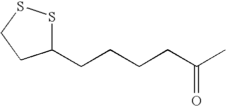 | Homophe | NH | 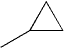 | | |
| 698 | 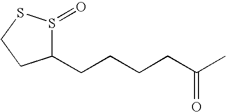 | Homophe | NH | 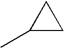 | | |
| 699 | 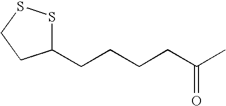 | Homophe | NH | CH₂COPh | | |
| 700 | 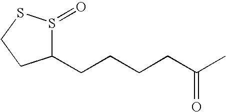 | Homophe | NH | CH₂COPh | | |
| 701 | 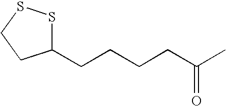 | Homophe | NH | 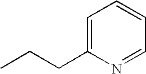 | | |
| 702 | 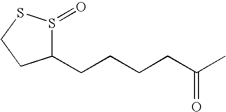 | Homophe | NH | 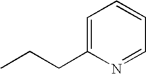 | | |
| 703 | 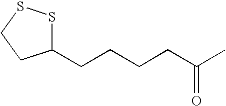 | Homophe | NH | 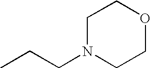 | | |
| 704 | 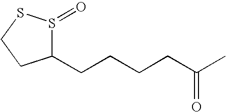 | Homophe | NH | 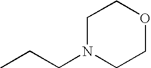 | | |
| 705 | 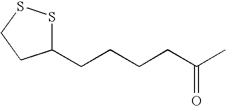 | Homophe | NH | CH₂CONH₂ | | |
| 706 | 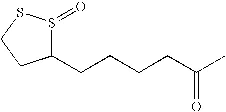 | Homophe | NH | CH₂CONH₂ | | |
| 707 | 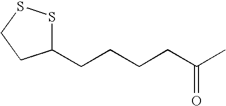 | Homophe | NH | CH₂COOEt | | |
| 708 | 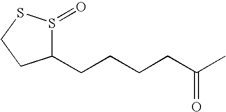 | Homophe | NH | CH₂COOEt | | |
| 709 | 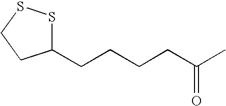 | Homophe | NH | CH₂COOH | | |
| 710 | 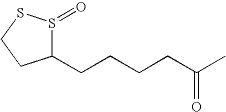 | Homophe | NH | CH₂COOH | | |
| 711 | 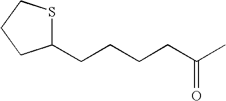 | Homophe | NH | Et | | |
| 712 | 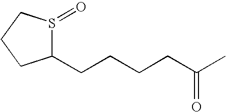 | Homophe | NH | Et | | |
| 713 | 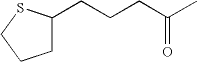 | Homophe | NH | Et | | |
| 714 | 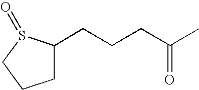 | Homophe | NH | Et | | |
| 715 | 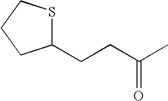 | Homophe | NH | Et | | |
| 716 | 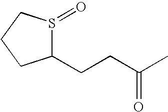 | Homophe | NH | Et | | |
| 717 | 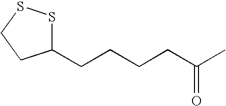 | Phe(4-F) | NH | Et | | |
| 718 | 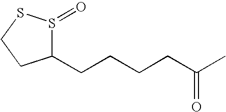 | Phe(4-F) | NH | Et | | |
| 719 | 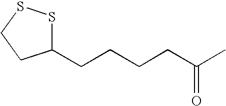 | Phe(4-Cl) | NH | Et | | |
| 720 | 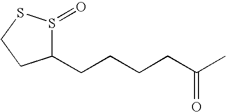 | Phe(4-Cl) | NH | Et | | |
| 721 | 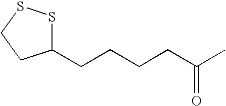 | Phe(3,4-Cl₂) | NH | Et | | |
| 722 | 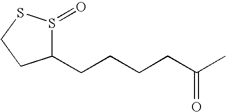 | Phe(3,4-Cl₂) | NH | Et | | |
| 723 | 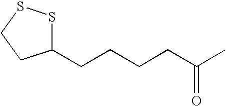 | Phe(4-OMe) | NH | Et | | |
| 724 | 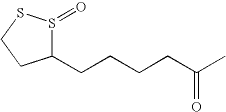 | Phe(4-OMe) | NH | Et | | |
| 725 | 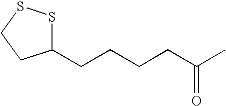 | 3-PyAla | NH | Et | | |
| 726 | 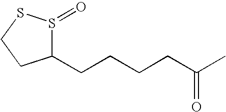 | 3-PyAla | NH | Et | | |
| 727 | 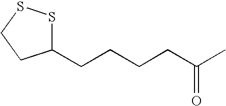 | 3-Benzo- thienylAla | NH | Et | | |
| 728 | 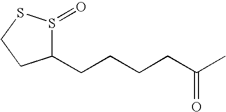 | 3-Benzo- thienylAla | NH | Et | | |
| 729 | 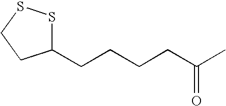 | CyclohexylAla | NH | Et | | |
| 730 | 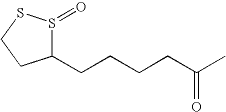 | CyclohexylAla | NH | Et | | |
| 731 | 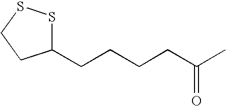 | Leu | NH | Et | | |
| 732 | 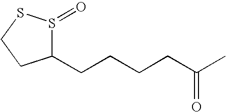 | Leu | NH | Et | | |

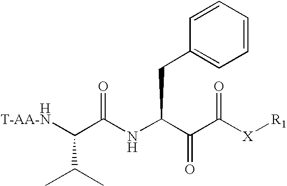

| | | | | | TLC | Mp. |
|---|---|---|---|---|---|---|
| Ex | T | AA | X | R₁ | [R_{f}(Solv.)] | [° C.] |
| 733 | 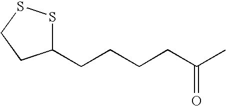 | Phe | NH | Et | 0.42 (CH₂Cl₂/MeOH 20:1) | 205 |
| 734 | 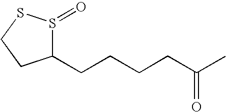 | Phe | NH | Et | | |
| 735 | 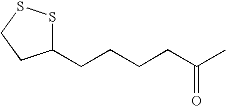 | 1-NaI | NH | Et | 0.57 (CH₂Cl₂/MeOH 20:1) | 220 |
| 736 | 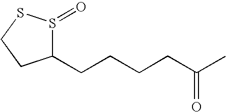 | 1-NaI | NH | Et | | |
| 737 | 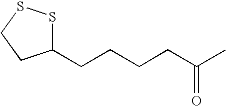 | 2-NaI | NH | Et | | |
| 738 | 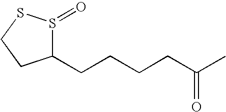 | 2-NaI | NH | Et | | |
| 739 | 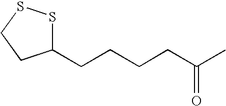 | Homophe | NH | Et | | |
| 740 | 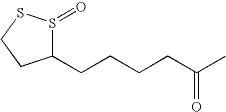 | Homophe | NH | Et | | |
| 741 | 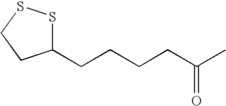 | Leu | NH | Et | 0.71 (CH₂Cl₂/MeOH 10:1) | 204 |
| 742 | 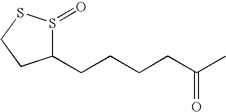 | Leu | NH | Et | | |

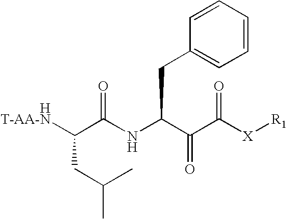

| | | | | | TLC | Mp. |
|---|---|---|---|---|---|---|
| Ex | T | AA | X | R₁ | [R_{f}(Solv.)] | [° C.] |
| 743 | 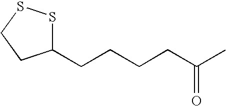 | Phe | NH | Et | | |
| 744 | 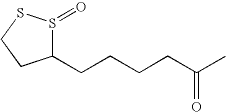 | Phe | NH | Et | | |
| 745 | 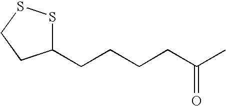 | 1-NaI | NH | Et | | |
| 746 | 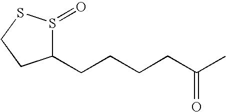 | 1-NaI | NH | Et | | |
| 747 | 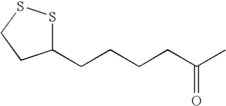 | 2-NaI | NH | Et | | |
| 748 | 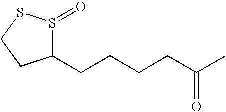 | 2-NaI | NH | Et | | |
| 749 | 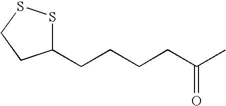 | Homophe | NH | Et | | |
| 750 | 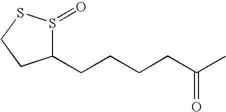 | Homophe | NH | Et | | |
| 751 | 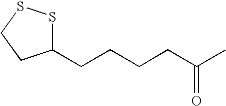 | Leu | NH | Et | | |
| 752 | 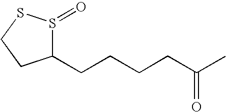 | Leu | NH | Et | | |

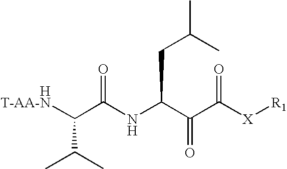

| | | | | | TLC | Mp. |
|---|---|---|---|---|---|---|
| Ex | T | AA | X | R₁ | [R_{f}(Solv.)] | [° C.] |
| 753 | 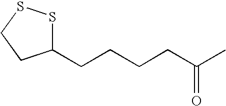 | Phe | NH | Et | | |
| 754 | 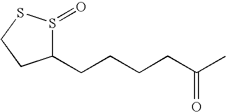 | Phe | NH | Et | | |
| 755 | 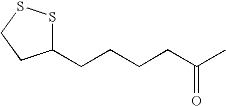 | 1-NaI | NH | Et | | |
| 756 | 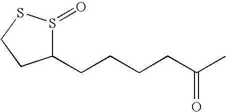 | 1-NaI | NH | Et | | |
| 757 | 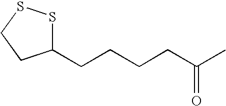 | 2-NaI | NH | Et | | |
| 758 | 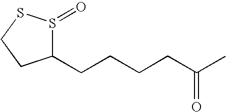 | 2-NaI | NH | Et | | |
| 759 | 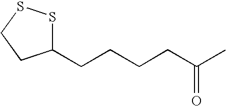 | Homophe | NH | Et | | |
| 760 | 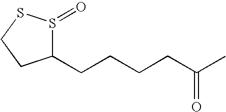 | Homophe | NH | Et | | |
| 761 | 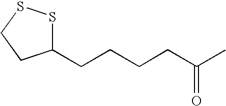 | Leu | NH | Et | | |
| 762 | 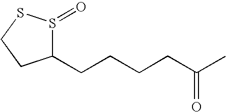 | Leu | NH | Et | | |

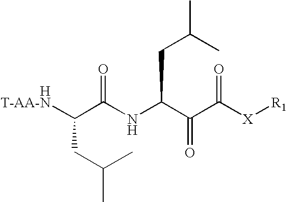

| | | | | | TLC | Mp. |
|---|---|---|---|---|---|---|
| Ex | T | AA | X | R₁ | [R_{f}(Solv.)] | [° C.] |
| 763 | 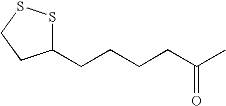 | Phe | NH | Et | 0.44 (CH₂Cl₂/MeOH 20:1) | 170 |
| 764 | 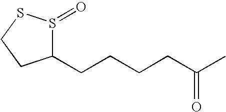 | Phe | NH | Et | | |
| 765 | 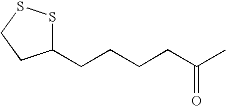 | 1-NaI | NH | Et | | |
| 766 | 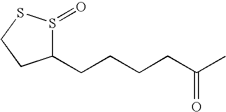 | 1-NaI | NH | Et | | |
| 767 | 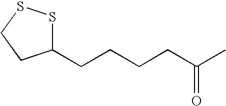 | 2-NaI | NH | Et | | |
| 768 | 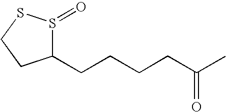 | 2-NaI | NH | Et | | |
| 769 | 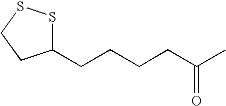 | Homophe | NH | Et | | |
| 770 | 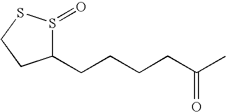 | Homophe | NH | Et | | |
| 771 | 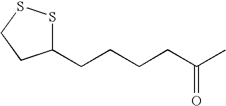 | Leu | NH | 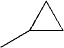 | | |
| 772 | 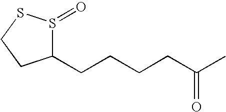 | Leu | NH | 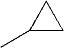 | | |
| 773 | 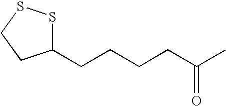 | Leu | NH | Et | 0.58 (CH₂Cl₂/MeOH 10:1) | 119-132 |
| 774 | 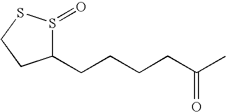 | Leu | NH | Et | 0.53 (CH₂Cl₂/MeOH 10:1) | 190 |

Biological Assays:

The inhibiting effect of the α-keto carbonyl calpain inhibitors of formula (I) was determined using enzyme tests which are customary in the literature, with the concentration of the inhibitor at which 50% of the enzyme activity is inhibited (=IC₅₀) being determined as the measure of efficacy. The Kᵢ value was also determined in some cases. These criteria were used to measure the inhibitory effect of the compounds (I) on calpain I, calpain II and cathepsin B.

Enzymatic Calpain Inhibition Assay

The inhibitory properties of calpain inhibitors are tested in 100 μl of a buffer containing 100 mM imidazole pH 7.5, 5 mM L-Cystein-HCl, 5 mM CaCl₂, 250 μM of the calpain fluorogenic substrate Suc-Leu-Tyr-AMC (Sigma) (Sasaki et al., J. Biol. Chem., 1984, 259, 12489-12949) dissolved in 2.5 μl DMSO and 0.5 μg of human μ-calpain (Calbiochem). The inhibitors dissolved in 1 μl DMSO are added to the reaction buffer. The fluorescence of the cleavage product 7-amino-4-methylcoumarin (AMC) is followed in a SPECTRAmax GEMINI XS (Molecular Device) fluorimeter at λₑₓ=360 nm and λₑₘ=440 nm at 30° C. during 30 min at intervals of 30 sec in 96-well plates (Greiner). The initial reaction velocity at different inhibitor concentrations is plotted against the inhibitor concentration and the IC₅₀ values determined graphically.

Calpain Inhibition Assay in C2C12 Myoblasts

This assay is aimed at monitoring the ability of the substance to inhibit cellular calpains. C2C12 myoblasts are grown in 96-well plates in growth medium (DMEM, 20% foetal calf serum) until they reach confluency. The growth medium is then replaced by fusion medium (DMEM, 5% horse serum). 24 hours later the fusion medium is replaced by fusion medium containing the test substances dissolved in 1 μl DMSO. After 2 hours of incubation at 37° C. the cells are loaded with the calpain fluorogenic substrate Suc-Leu-Tyr-AMC at 400 μM in 50 μl of a reaction buffer containing 135 mM NaCl; 5 mM KCl; 4 mM CaCl₂; 1 mM MgCl₂; 10 mM Glucose; 10 mM HEPES pH 7.25 for 20 min at room temperature. The calcium influx, necessary to activate the cellular calpains, is evoked by the addition of 50 μl reaction buffer containing 20 μM of the calcium ionophore Br-A-23187 (Molecular Probes). The fluorescence of the cleavage product AMC is measured as described above during 60 min at 37° C. at intervals of 1 min. The IC₅₀ values are determined as described above. Comparison of the IC₅₀ determined in the enzymatic calpain inhibition assay to the IC₅₀ determined in the C2C12 myoblasts calpain inhibition assay, allows to evaluate the cellular uptake or the membrane permeability of the substance.

Spectrin Breakdown Assay in C2C12 Myoblasts

Although calpains cleave a wide variety of protein substrates, cytoskeletal proteins seem to be particularly susceptible to calpain cleavage. Specifically, the accumulation of calpain-specific breakdown products (BDP's) of the cytoskeletal protein alpha-spectrin has been used to monitor calpain activity in cells and tissues in many physiological and pathological conditions. Thus, calpain activation can be measured by assaying the proteolysis of the cytoskeletal protein alpha-spectrin, which produces a large (150 kDa), distinctive and stable breakdown product upon cleavage by calpains (A. S. Harris, D. E. Croall, & J. S. Morrow, The calmodulin-binding site in alpha-fodrin is near the calcium-dependent protease-1 cleavage site, J. Biol. Chem., 1988, 263(30), 15754-15761. Moon, R. T. & A. P. McMahon, Generation of diversity in nonerythroid spectrins. Multiple polypeptides are predicted by sequence analysis of cDNAs encompassing the coding region of human nonerythroid alpha-spectrin, J. Biol. Chem., 1990, 265(8), 4427-4433. P. W. Vanderklish & B. A. Bahr, The pathogenic activation of calpain: a marker and mediator of cellular toxicity and disease states, Int. J. Exp. Pathol., 2000, 81(5), 323-339). The spectrin breakdown assay is performed under the same conditions as in the C2C12 myoblast calpain inhibition assay described above, except that the fluorogenic substrate is omitted. After the 60 min incubation with the calcium ionophore, the cells are lysed in 50 μl of lysis buffer containing 80 mM Tris-HCl pH 6.8; 5 mM EGTA; 2% SDS. The lysates are then probed on western blots using the monoclonal antibody mAb1622 (Chemicon). The activation of calpains is determined by measuring the ratio of the 150 kDa calpain-specific BDP to the intact 240 kDa alpha-spectrin band densitometrically.

Cathepsin B Assay

Inhibition of cathepsin B was determined by a method which was similar to a method of S. Hasnain et al., J. Biol. Chem., 1993, 268, 235-240. 2 μL of an inhibitor solution, prepared from inhibitor and DMSO (final concentrations: 100 μM to 0.01 μM) are added to 88 μL of cathepsin B (human liver cathepsin B (Calbiochem) diluted to 5 units in 500 μM buffer). This mixture is preincubated at room temperature (25° C.) for 60 min and the reaction is then starting by adding 10 μL of 10 mM Z-Arg-Arg-pNA (in buffer containing 10% DMSO). The reaction is followed at 405 nm for 30 min in a microtiter plate reader. The IC₅₀'s are then determined from the maximum slopes.

20S Proteasome Assay

25 μl of a reaction buffer containing 400 μM of the fluorogenic substrate Suc-Leu-Leu-Val-Tyr-AMC (Bachem) are dispensed per well of a white microtiter plate. Test compounds dissolved in 0.5 μl DMSO are added. To start the reaction; 25 μl of reaction buffer containing 35 ng of enzyme (20S Proteasome, Rabbit, Calbiochem) are added. The increase in fluorescence (excitation at 360 nm; emission at 440 nm) is measured over 30 min at 30° C. at 30″. The IC₅₀'s are then determined from the slopes.

BSO Assay

Primary fibroblasts were derived from donors with molecular diagnosis for Friedreich Ataxia (FRDA) and control donors with no mitochondrial disease. Cell lines were obtained from Coriell Cell Repositories (Camden, N.J.; catalog numbers GM04078, GM08402 and GM08399 respectively). All cell types were diagnosed on the molecular level for intronic GM triplet repeat length of at least 400-450 repeats using a PCR-based method. Experiments were carried out as described in the literature (M. L. Jauslin et al., Human Mol. Genet., 2002, 11, 3055-3063): Cells were seeded in microtiter plates at a density of 4'000 cells per 100 μl in growth medium consisting of 25% (v/v) M199 EBS and 64% (v/v) MEM EBS without phenol red (Bioconcept, Allschwil, Switzerland) supplemented with 10% (v/v) fetal calf serum (PAA Laboratories, Linz, Austria), 100 U/ml penicillin, 100 μg/ml streptomycin (PAA Laboratories, Linz, Austria), 10 μg/ml insulin (Sigma, Buchs, Switzerland), 10 ng/ml EGF (Sigma, Buchs, Switzerland), 10 ng/ml bFGF (PreproTech, Rocky Hill, N.J.) and 2 mM glutamine (Sigma, Buchs, Switzerland). The cells were incubated in the presence of various test compounds for 24 h before addition of L-buthionine-(S,R)-sulfoximine (BSO) to a final concentration of 1 mM. Cell viability was measured after the first signs of toxicity appeared in the BSO-treated controls (typically after 16 to 48 h). The cells were stained for 60 min at room temperature in PBS with 1.2 μM calceinAM and 4 μM ethidium homodimer (Live/Dead assay, Molecular Probes, Eugene, Oreg.). Fluorescence intensity was measured with a Gemini Spectramax XS spectrofluorimeter (Molecular Devices, Sunnyvale, Calif.) using excitation and emission wavelengths of 485 nm and 525 nm respectively.

Utrophin Expression Assay in Human Myotubes

Utrophin induction was determined by a method which was similar to a method of 1. Courdier-Fruh et al., Neuromuscular Disord., 2002, 12, S95-S104. Primary human muscle cell cultures were prepared from muscle biopsies taken during orthopedic surgery from Duchenne patients (provided by the Association Francaise contre les Myopathies). Cultures were prepared and maintained according to standard protocols. Induction of utrophin expression in human DMD myotubes was assayed at 50 nM or 500 nM of test compound added in differentiation medium. Normalized utrophin protein levels are determined after 56 d of incubation by cell-based ELISA with a mouse monoclonal antibody to the aminoterminal portion of utrophin (NCL-DRP2, Novocastra Laboratories). For calibration, the cell density and differentiation was determined by absorbance measurements of the total dehydrogenase enzyme activity in each well using the calorimetric CellTiter 96®AQ One Solution Reagent Proliferation Assay (Promega) according to the manufacturer's recommendation. Subsequently, cells were fixed, washed, permeabilized with 0.5% (v/v) Triton X-100 and unspecific antibody binding-sites blocked by standard procedures. Utrophin protein levels were determined immunologically with utrophin-specific primary antibody and with an appropriate peroxidase-coupled secondary antibody (Jackson ImmunoResearch Laboratories) using QuantaBlu™ Fluorogenic Peroxidase Substrate Kit (Pierce) for detection. Fluorescence measurements were carried out with a multilabel counter (Wallac) at λₑₓ=355 nm and at λₑₘ=460 nm. The primary readout of this signal is presented in arbitrary units. For calibration, the arbitrary units representing the relative utrophin protein content of each well was divided by the corresponding cell-titer absorbance value to correct for cell density. For comparison between experiments, the cell-titer corrected readout for utrophin protein content in each well was expressed in percent of solvent treated control cultures (set to 100%), i.e. data are % utrophin protein levels compared to DMSO solvent (N=4).

Biological Data for selected Examples of the Invention:

| | | Calp I | | | |
|---|---|---|---|---|---|
| | Calp I | IC₅₀ | 20S Prot | BSO | UTR |
| | IC₅₀ | Myoblast | IC₅₀ | EC₅₀ | Induction |
| Example | μM | μM | μM | μM | @50 nM |
| MDL-28170 | 0.020 | 40.000 | >1 | n.d. | n.d. |
| 1 | 0.045 | 0.200 | n.d. | n.d. | n.d. |
| 2 | 0.038 | 0.210 | 0.16 | 0.80 | n.d. |
| 606 | 0.016 | 0.028 | 0.010 | n.d. | 151% |
| 637 | 0.030 | 0.040 | 0.040 | n.d. | 121% |
| 735 | 0.035 | 0.020 | 0.027 | n.d. | 134% |

Examples with an IC₅₀ in the Calpain Inhibition Assay in C2C12 Myoblasts of 1 μM or lower generally exhibited complete inhibition of Spectrin Breakdown in C2C12 myoblasts at a test concentration of 10 μM.

In vivo Experiments:

The mdx mouse is a well established animal model for Duchenne Muscular Dystrophy (Bulfield G., Siller W. G., Wight P. A., Moore K. J., X chromosome-linked muscular dystrophy (mdx) in the mouse, Proc. Natl. Acad. Sci. USA., 1984, 81(4), 1189-1192). Selected compounds were tested in longterm treatments of mdx mice, according to the procedures described below.

Mouse strains: C57BL/10scsn and C57BL/10scsn mdx mouse strains were purchased at The Jackson Laboratory (ME, USA) and bred inhouse. Mouse males were sacrificed at the age of 3 or 7 weeks by CO₂ asphyxiation.

Treatment: Compounds were dissolved in 50% PEG, 50% saline solution and applied by i.p. injection.

Histology: Tibialis anterior (TA), quadriceps (Quad), and diaphragm (Dia) muscles were collected and mounted on cork supports using gum tragacanth (Sigma-Aldrich, Germany). The samples were snap-frozen in melting isopentane and stored at −80° C. 12 μm thick cryosections of the mid-belly region of muscles were prepared. For staining, sections were air dried and fixed with 4% PFA in PBS for 5 minutes, washed 3 times with PBS and incubated over night at 4° C. in PBS containing 2 μg/ml Alexa Fluor ™ 488 conjugated wheat-germ agglutinin (WGA-Alexa, Molecular Probes, Eugene, Oreg., USA) to stain membrane-bound and extracellular epitopes and 1 μg/ml 4′,6-diamidino-2-phenylindole (DAPI; Molecular Probes) to stain nuclei.

Image acquisition and analysis: Fluorescence microscopy images of both labels were acquired using a digital camera (ColorView II, Soft Imaging System, Münster, Germany) coupled to a fluorescence microscope (Vanox S, Olympus, Tokyo, Japan). Combination of these two stainings to a composite image, assembling of several images to a complete image of the entire muscle cross-section and further semi-automated analysis was performed using the image analysis program AnalySIS (Soft Imaging System). Image analysis of 1200-2900 muscle fibers in each section was performed in three steps: 1) determination of the muscle fiber boundaries, 2) determination of the muscle fiber size, and 3) determination of the percentage of muscle fibers containing centralized nuclei. Six different geometrical parameters were tested for the determination of the muscle fiber size: (a) the “minimal feret” (the minimum distance of parallel tangents at opposing borders of the muscle fiber), (b) the “area”, (c) the “minimal inner diameter” (the minimum diameter through the center of the muscle fiber), (d) the “minimal diameter” (the minimum diameter of a muscle fiber for angles in the range 0° through 179° with step width 1°, (e) the “minimal outer diameter” (the minimum diameter through the muscle fiber from outer border to outer border), and (f) the “perimeter”. The variance coefficient of the muscle fiber size is defined as follows: variance coefficient=(standard deviation of the muscle fiber size/mean of the muscle fiber size of the section)*1000. For statistical analysis of different variance coefficient values Mann-Whitney U test was used.

Selected Examples of the present invention were active in the mdx mouse model at 20 mg/kg every 2^{nd} day, using 3 week old mice and a treatment period of 4 weeks (N=5−10).

Example 2 at 20 mg/kg every 2^{nd} day lead to a decrease in the variance coefficient of the muscle fiber size by 26% (p<0.05; N=5) in the Dia, compared to control mdx mice receiving vehicle only (N=15).

No prominent adverse effects of the compound were observed upon this longterm treatment.

Example 637 at 20 mg/kg every 2^{nd} day lead to a decrease in the variance coefficient of the muscle fiber size by 34% (p<0.0005; N=8) in the Dia, and by 32% (p<0.05; N=3) in the Quad, compared to control mdx mice receiving vehicle only (N=15). The precentage of centralized nuclei was reduced by 34% (p<0.01; N=8) in the Dia, compared to control mdx mice receiving vehicle only (N=20). Example 637 at 2 mg/kg every 2^{nd} day lead to a decrease in the variance coefficient of the muscle fiber size by 33% (p<0.005; N=5) in the Dia, compared to control mdx mice receiving vehicle only (N=15).

No prominent adverse effects of the compound were observed upon these longterm treatments.

In contrast to this, the potent standard calpain inhibitor MDL-28170 showed only weak activity in this experiment.

As evident from the results presented above, generally compounds of the present invention display significantly improved activity in C2C12 muscle cells compared to standard calpain inhibitors such as MDL-28170. For selected examples the improvement in the cellular assay is in excess of a factor of thousand, whereas their activity in the enzymatic calpain I inhibition assay is comparable to the one of MDL-28170.

This illustrates that the compounds of the present invention possess greatly enhanced muscle cell membrane permeability with regard to the known standard compound MDL-28170, while retaining the potent activity for inhibition of calpain. This improved cell penetration renders them particularly useful for the treatment of diseases, where the site of action is a muscle tissue, such as muscular dystrophy and amyotrophy.

As illustrated by the biological results (see above), in addition to showing potent calpain inhibitory activity, selected examples of the present invention are also potent inhibitors of the proteasome (MCP) and/or effectively protect muscle cells from damage due to oxidative stress and/or induce the expression of utrophin. Such additional beneficial properties could be advantageous for treating certain muscular diseases such as muscular dystrophy and amyotrophy.

In contrast to known calpain inhibitors of the peptide aldehyde class, such as MDL-28170, the compounds of the present invention possess the necessary metabolic stability and physicochemical properties to permit their successful application in vivo. Selected compounds of the present invention accordingly exhibited potent activity upon longterm treatment in a mouse model of Duchenne Muscular Dystrophy, whereas the activity of standard calpain inhibitory aldehydes, e.g. MDL-28170 in this animal model was weak.

### EXAMPLES OF A PHARMACEUTICAL COMPOSITION

As a specific embodiment of an oral composition of the present invention, 80 mg of the compound of Example 1 is formulated with sufficient finely divided lactose to provide a total amount of 580 to 590 mg to fill a size 0 hard gelatin capsule.

While the invention has been described and illustrated in reference to certain preferred embodiments thereof, those skilled in the art will appreciate that various changes, modifications and substitutions can be made therein without departing from the scope of the invention. For example, effective dosages other than the preferred doses as set forth hereinabove may be applicable as a consequence of the specific pharmacological responses observed and may vary depending upon the particular active compound selected, as well as from the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

1. A compound of structural formula (I): 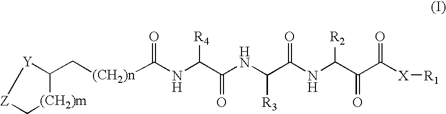
or a pharmaceutically acceptable salt or solvate thereof, wherein
R¹ represents
hydrogen,
straight chain alkyl,
branched chain alkyl,
cycloalkyl,
-alkylene-cycloalkyl,
aryl,
-alkylene-aryl,
—SO₂-alkyl,
—SO₂-aryl,
-alkylene-SO₂-aryl,
-alkylene-SO₂-alkyl,
heterocyclyl or
-alkylene-heterocyclyl;
—CH₂CO—X—H
—CH₂CO—X-straight chain alkyl,
—CH₂CO—X-branched chain alkyl,
—CH₂CO—X-cycloalkyl,
—CH₂CO—X-alkylene-cycloalkyl,
—CH₂CO—X-aryl,
—CH₂CO—X-alkylene-aryl,
—CH₂CO—X-heterocyclyl,
—CH₂CO—X-alkylene-heterocyclyl or
—CH₂CO-aryl;
X represents O or NH;
R² represents
hydrogen,
straight chain alkyl,
branched chain alkyl,
cycloalkyl,
-alkylene-cycloalkyl,
aryl or
-alkylene-aryl;
R³ represents
hydrogen,
straight chain alkyl,
branched chain alkyd,
cycloalkyl,
-alkylene-cycloalkyl or
-alkenylene-aryl;
R⁴ represents
straight chain alkyl,
branched chain alkyl,
cycloalkyl,
-alkylene-cycloalkyl,
aryl or
-alkylene-aryl;
wherein each of m and n represents an integer of 0 to 6, i.e. 1, 2, 3, 4, 5 or 6;
Y and Z independently represents
S.
SO or
CH₂.

2. The compound of claim 1, wherein R¹ is selected from the group consisting of hydrogen, straight chain alkyl, branched chain alkyl, cycloalkyl, -alkylene-aryl, -alkylene-heterocyclyl, —CH₂CO—X-straight chain alkyl, —CH₂COOH, and —CH₂CONH₂.

3. The compound of claim 1, wherein R² is a substituted or unsubstituted benzyl group.

4. The compound of claim 1, wherein R³ is a branched chain alkyl group, a cycloalkyl group or an -alkylene-cycloalkyl group.

5. The compound of claim 1, wherein R⁴ is a substituted or unsubstituted benzyl or ethylphenyl group.

6. The compound of claim 1, wherein R⁴ is a methylnaphthyl group.

7. The compound of claim 1, wherein m=1, n=3, and Y and Z are both S or Y is S and Z is SO or Y is SO or Z is S.

8. The compound of claim 1, wherein m=1, n=3, and Y and Z are both S.

9. The compound of claim 1 for use as a medicament.

10. A method for the treatment or prevention of disorders, diseases or conditions responsive to the inhibition of calpain I or other thiol proteases comprising administering to a subject said compound of claim 1 or a pharmaceutically acceptable salt or solvate thereof.

11. The method according to claim 10 wherein the treatment or prevention is for the treatment or prevention of disorders, diseases or conditions responsive to the inhibition of cathepsin B, cathepsin H, cathepsin L, or papain.

12. The method according to claim 10 wherein the treatment or prevention is for the treatment or prevention of disorders, diseases or conditions responsive to the inhibition of Multicatalytic Protease (MCP).

13. The method according to claim 10 wherein the treatment or prevention is for the treatment or prevention of Duchenne Muscular Dystrophy (DMD).

14. The method according to claim 10 wherein the treatment or prevention is for the treatment or prevention of Becker Muscular Dystrophy (BMD).

15. The method according to claim 10 wherein the treatment or prevention is for the treatment or prevention of neuromuscular diseases.

16. The method according to claim 15 wherein the treatment or prevention is for the treatment or prevention of muscular dystrophies, including dystrophinopathies and sarcoglycanopathies, limb girdle muscular dystrophies, congenital muscular dystrophies, congenital myopathies, distal and other myopathies, myotonic syndromes, ion channel diseases, malignant hyperthermia, metabolic myopathies, hereditary cardiomyopathies, congenital myasthenic syndromes, spinal muscular atrophies, hereditary ataxias, hereditary motor and sensory neuropathies wad or hereditary paraplegias.

17. The method according to claim 10 wherein the treatment or prevention is for the treatment or prevention of disuse atrophy or general muscle wasting.

18. The method according to claim 10 wherein the treatment or prevention is for the treatment or prevention of ischemias of the heart, of the kidney or of the central nervous system, inflammations, muscular dystrophies, injuries to the central nervous system or Alzheimer's disease.

19. The method according to claim 10 wherein the treatment or prevention is for the treatment or prevention cataracts of the eye, or other diseases of the eye.

20. The method according to claim 12 wherein the treatment or prevention is for the treatment of cancer.

21. The method according to claim 12 wherein the treatment or prevention is for the treatment of psoriasis, or restenosis, or other cell proliferative diseases.

22. A method for the treatment or prevention of mitochondrial disorders or neurodegenerative diseases, where elevated levels of oxidative stress are involved comprising administering to a subject said compound of claim 1 or a pharmaceutically acceptable salt or solvate thereof.

23. The method according to claim 22 wherein the treatment or prevention is for the treatment of mitochondrial disorders including, Kearns-Sayre syndrome, mitochondrial encephalomyopathy-lactic-acidosis-stroke like episodes (MELAS), myoclonic epilepsy and ragged-red-fibers (MERRF), Leber hereditary optic neuropathy (LHON), Leigh's syndrome, neuropathy-ataxia-retinitis pigmentosa (NARP) or progressive external opthalmoplegia (PEO).

24. The method according to claim 22 wherein the treatment or prevention is for the treatment of neurodegenerative diseases with free radical involvement including degenerative ataxias such as Friedreich' Ataxia, Parkinson's disease, Huntington's disease, amyotrophic lateral sclerosis (ALS) or Alzheimer's disease.

25. A method for the treatment or prevention of disorders, diseases or conditions responsive to induction of utrophin expression comprising administering to a subject said compound of claim 1 or a pharmaceutically acceptable salt or solvate thereof.

26. The method according to claim 25 wherein the treatment or prevention is for the treatment or prevention of Duchenne Muscular Dystrophy (DMD).

27. The method according to claim 25 wherein the treatment or prevention is for the treatment or prevention of Becker Muscular Dystrophy (BMD).

28. A pharmaceutical composition which comprises a compound of claim 1 and a pharmaceutically acceptable carrier.
